SEQUENCE LISTING (41 sequences)

SEQ ID NO: 1 - DNA, 940 bp, Homo sapiens
  CDS (138)..(239)
  sig_peptide (138)..(227)
  mat_peptide (228)..()
  Intron (240)..(440)
  CDS (442)..(513)
  Intron (515)..(691)
  CDS (692)..(811)
  mutation (760)..(760): G to C SNP
gagactgctggccctgccccagggcaggtgctgacgcaggcttggaatgaaggccctttgtgaggtggccctgggagccggcaacgggttccgtcctgccagcatagggacgacaggagccaggacccgtgtaggagatggttctcgccatgctgggggctctgcacccccgggctgggctcagcctcttcctccacctcatcctggcagtggcactgcttcgctcccagcctctgaggtgactcctggggcacagagggcagggcacttgggccggatggtggaggtgctgcccctcccctctctggagtggggctggccctggtaccctgggcgttcgagggcttgccaagtgcccccacactgagccatccccccattcacaaggaggtggcggtggccttctgggccagggctgagcaccccgacatctcccacaggtctcagcggtctgttcctgaggcattttccgcccccctggaactctcgcagccactttccggcctggtggatggtagtatctctcttgtccccttcccctacccatccctgacagtggagacgcccctaagaggtgccccaagtccagccttcaggggcttgtcatcccaaaccacatctgttcaagggtgacttcagttccatcagagccagctgccccctaccacccatgccctcttttctctctttagactatggcatcctccccaagcacccaaggccgcgagggcctcgacccctcctgtctagggcccagcagcgcaagcgggacgggcccgaccttgccgagtattactatgatgcacacctatgacccaaggccctcataaagataccatgtgtgacaaaggcttggctttcctgggctgggagcgagacggggacgggaggggcccatgcagatacatccactgcagtccagacaggaaatggcacttta

SEQ ID NO: 2 - protein, 98 aa, Homo sapiens
MVLAMLGALHPRAGLSLFLHLILAVALLRSQPLRSQRSVPEAFSAPLELSQPLSGLVDDYGILPKHPRPRGPRPLLSRAQQRKRDGPDLAEYYYDAHL

SEQ ID NO: 3 - DNA, 360 bp, Homo sapiens
  CDS (38)..(331)
  sig_peptide (38)..(127)
  variation (50)..(52): Alternative start codon
agcatagggacgacaggagccaggacccgtgtaggagatggttctcgccatgctgggggctctgcacccccgggctgggctcagcctcttcctccacctcatcctggcagtggcactgcttcgctcccagcctctgaggtctcagcggtctgttcctgaggcattttccgcccccctggaactctcgcagccactttccggcctggtggatgactatggcatcctccccaagcacccaaggccgcgagggcctcgacccctcctgtctagggcccagcagcgcaagcgggacgggcccgaccttgccgagtattactatgatgcacacctatgacccaaggccctcataaagataccatg

SEQ ID NO: 4 - protein, 98 aa, Homo sapiens; identical to SEQ ID NO: 2

SEQ ID NO: 5 - protein, 94 aa, Homo sapiens
  SIGNAL (1)..(26)
  mat_peptide (27)..()
MLGALHPRAGLSLFLHLILAVALLRSQPLRSQRSVPEAFSAPLELSQPLSGLVDDYGILPKHPRPRGPRPLLSRAQQRKRDGPDLAEYYYDAHL

SEQ ID NO: 6 - DNA, 360 bp, Homo sapiens
  CDS (38)..(331)
  sig_peptide (38)..(127)
  variation (50)..(52): Alternative start codon
agcatagggacgacaggagccaggacccgtgtaggagatggttctcgccatgctgggggctctgcacccccgggctgggctcagcctcttcctccacctcatcctggcagtggcactgcttcgctcccagcctctgaggtctcagcggtctgttcctgaggcattttccgcccccctggaactctcgcagccactttccggcctggtggatgactatggcatcctccccaagcacccaaggccgcgagggcctcgacccctcctgtctagggcccagcaccgcaagcgggacgggcccgaccttgccgagtattactatgatgcacacctatgacccaaggccctcataaagataccatg

SEQ ID NO: 7 - protein, 98 aa, Homo sapiens
MVLAMLGALHPRAGLSLFLHLILAVALLRSQPLRSQRSVPEAFSAPLELSQPLSGLVDDYGILPKHPRPRGPRPLLSRAQHRKRDGPDLAEYYYDAHL

SEQ ID NO: 8 - protein, 94 aa, Homo sapiens
  SIGNAL (1)..(26)
  mat_peptide (27)..()
MLGALHPRAGLSLFLHLILAVALLRSQPLRSQRSVPEAFSAPLELSQPLSGLVDDYGILPKHPRPRGPRPLLSRAQHRKRDGPDLAEYYYDAHL

SEQ ID NO: 9 - protein, 68 aa, Homo sapiens
QPLRSQRSVPEAFSAPLELSQPLSGLVDDYGILPKHPRPRGPRPLLSRAQQRKRDGPDLAEYYYDAHL

SEQ ID NO: 10 - protein, 68 aa, Homo sapiens
QPLRSQRSVPEAFSAPLELSQPLSGLVDDYGILPKHPRPRGPRPLLSRAQHRKRDGPDLAEYYYDAHL

SEQ ID NO: 11 - DNA, 363 bp, Mus musculus
  CDS (32)..(328)
  sig_peptide (32)..(118)
  misc_feature (44)..(47): Alternative start codon
gagatagcccccacagcagggactggagacaatggttctcgccatgcttggggctctgtaccccagggctggactcagtctcttccttttttacctcatcttggcaggcgcactccttcgacctcagccccagaggtctcagcaatctgttccggaggaattttcagccccgctggaactgttgcagcctctctcaggcctggtggatgactatgggctcagacccaagcacccgaggccaggtgggccccgaccccttctctcccaggcccagcagcgcaagcgggacgggcccaacatggctgactattactatgatgtgaacctgtgatgggaaccccgcataaagctattctgggcagc

SEQ ID NO: 12 - protein, 99 aa, Mus musculus
MVLAMLGALYPRAGLSLFLFYLILAGALLRPQPQRSQQSVPEEFSAPLELLQPLSGLVDDYGLRPKHPRPGGPRPLLSQAQQRKRDGPNMADYYYDVNL

SEQ ID NO: 13 - protein, 95 aa, Mus musculus
  SIGNAL (1)..(25)
  mat_peptide (26)..()
MLGALYPRAGLSLFLFYLILAGALLRPQPQRSQQSVPEEFSAPLELLQPLSGLVDDYGLRPKHPRPGGPRPLLSQAQQRKRDGPNMADYYYDVNL

SEQ ID NO: 14 - protein, 70 aa, Mus musculus
RPQPQRSQQSVPEEFSAPLELLQPLSGLVDDYGLRPKHPRPGGPRPLLSQAQQRKRDGPNMADYYYDVNL

SEQ ID NO: 15 - DNA, 300 bp, Rattus norvegicus
  CDS (1)..(300)
  sig_peptide (1)..(87)
  misc_feature (12)..(14): Alternative start codon
atggttctcgccatgcttggggttctgtaccccagggctgggctcagtctctttctattttacctcatcctggcaggcgcactccttcgacctcagccccagaggtctcagcgatctgttcctgaggaattttcagctccgctggaactgttgcagcctctctcaggcctggtggatgactacgggctaagacccaagcacccgaggccaggtgggcccagaccccttctctcccaggcccagcagcgcaagcgggacgggcccgacatggctgactattactatgatgtgaaccccgca

SEQ ID NO: 16 - protein, 100 aa, Rattus norvegicus
MVLAMLGVLYPRAGLSLFLFYLILAGALLRPQPQRSQRSVPEEFSAPLELLQPLSGLVDDYGLRPKHPRPGGPRPLLSQAQQRKRDGPDMADYYYDVNPA

SEQ ID NO: 17 - protein, 96 aa, Rattus norvegicus
  SIGNAL (1)..(25)
  mat_peptide (26)..()
MLGVLYPRAGLSLFLFYLILAGALLRPQPQRSQRSVPEEFSAPLELLQPLSGLVDDYGLRPKHPRPGGPRPLLSQAQQRKRDGPDMADYYYDVNPA

SEQ ID NO: 18 - protein, 71 aa, Rattus norvegicus
RPQPQRSQRSVPEEFSAPLELLQPLSGLVDDYGLRPKHPRPGGPRPLLSQAQQRKRDGPDMADYYYDVNPA

SEQ ID NO: 19 - DNA, 204 bp, Homo sapiens
cagcctctgaggtctcagcggtctgttcctgaggcattttccgcccccctggaactctcgcagccactttccggcctggtggatgactatggcatcctccccaagcacccaaggccgcgagggcctcgacccctcctgtctagggcccagcagcgcaagcgggacgggcccgaccttgccgagtattactatgatgcacaccta

SEQ ID NO: 20 - DNA, 204 bp, Homo sapiens
cagcctctgaggtctcagcggtctgttcctgaggcattttccgcccccctggaactctcgcagccactttccggcctggtggatgactatggcatcctccccaagcacccaaggccgcgagggcctcgacccctcctgtctagggcccagcaccgcaagcgggacgggcccgaccttgccgagtattactatgatgcacaccta

SEQ ID NO: 21 - DNA, 210 bp, Mus musculus
cgacctcagccccagaggtctcagcaatctgttccggaggaattttcagccccgctggaactgttgcagcctctctcaggcctggtggatgactatgggctcagacccaagcacccgaggccaggtgggccccgaccccttctctcccaggcccagcagcgcaagcgggacgggcccaacatggctgactattactatgatgtgaacctg

SEQ ID NO: 22 - DNA, 213 bp, Rattus norvegicus
cgacctcagccccagaggtctcagcgatctgttcctgaggaattttcagctccgctggaactgttgcagcctctctcaggcctggtggatgactacgggctaagacccaagcacccgaggccaggtgggcccagaccccttctctcccaggcccagcagcgcaagcgggacgggcccgacatggctgactattactatgatgtgaaccccgca

SEQ ID NO: 23 - protein, 99 aa, Pan troglodytes
  SIGNAL (1)..(30)
  mat_peptide (31)..()
MVLAMLGALHPRAGLSLFLHLILAVALLRSQPLRRSQRSVPEAFSAPLELSQPLSGLVDDYGILPKHPRPQGPRPLLSRAQQRKRDGPDLAEYYYDAHL

SEQ ID NO: 24 - protein, 95 aa, Pan troglodytes
  SIGNAL (1)..(26)
  mat_peptide (27)..()
MLGALHPRAGLSLFLHLILAVALLRSQPLRRSQRSVPEAFSAPLELSQPLSGLVDDYGILPKHPRPQGPRPLLSRAQQRKRDGPDLAEYYYDAHL

SEQ ID NO: 25 - protein, 69 aa, Pan troglodytes
QPLRRSQRSVPEAFSAPLELSQPLSGLVDDYGILPKHPRPQGPRPLLSRAQQRKRDGPDLAEYYYDAHL

SEQ ID NO: 26 - protein, 38 aa, Homo sapiens
QPLRSQRSVPEAFSAPLELSQPLSGLVDDYGILPKHPR

SEQ ID NO: 27 - protein, 40 aa, Homo sapiens
RSQPLRSQRSVPEAFSAPLELSQPLSGLVDDYGILPKHPR

SEQ ID NO: 28 - protein, 30 aa, Homo sapiens
PRGPRPLLSRAQQRKRDGPDLAEYYYDAHL

SEQ ID NO: 29 - protein, 30 aa, Homo sapiens
PRGPRPLLSRAQHRKRDGPDLAEYYYDAHL

SEQ ID NO: 30 - protein, 40 aa, Mus musculus
RPQPQRSQQSVPEEFSAPLELLQPLSGLVDDYGLRPKHPR

SEQ ID NO: 31 - protein, 30 aa, Mus musculus
PGGPRPLLSQAQQRKRDGPNMADYYYDVNL

SEQ ID NO: 32 - protein, 40 aa, Rattus norvegicus
RPQPQRSQRSVPEEFSAPLELLQPLSGLVDDYGLRPKHPR

SEQ ID NO: 33 - protein, 31 aa, Rattus norvegicus
PGGPRPLLSQAQQRKRDGPDMADYYYDVNPA

SEQ ID NO: 34 - protein, 39 aa, Pan troglodytes
QPLRRSQRSVPEAFSAPLELSQPLSGLVDDYGILPKHPR

SEQ ID NO: 35 - protein, 30 aa, Pan troglodytes
PQGPRPLLSRAQQRKRDGPDLAEYYYDAHL

SEQ ID NO: 36 - protein, 14 aa, Homo sapiens
QPLRSQRSVPEAFS

SEQ ID NO: 37 - protein, 15 aa, Homo sapiens
QQRKRDGPDLAEYYY

SEQ ID NO: 38 - protein, 14 aa, Homo sapiens
DGPDLAEYYYDAHL

SEQ ID NO: 39 - DNA, 294 bp, Pan troglodytes
  CDS (1)..(294)
atggttctcgccatgctgggggctctgcacccccgggctgggctcagcctcttcctccacctcatcctggcagtggcactgcttcgctcccagcctctgaggtctcagcggtctgttcctgaggcattttccgcccctctggaactctcgcagccactttccggcctggtggatgactatggcatcctccccaagcacccaaggccgcaagggcctcgacccctcctgtctagggcccagcagcgcaagcgggacgggcccgaccttgccgagtattactatgatgcacaccta

SEQ ID NO: 40 - protein, 98 aa, Pan troglodytes
MVLAMLGALHPRAGLSLFLHLILAVALLRSQPLRSQRSVPEAFSAPLELSQPLSGLVDDYGILPKHPRPQGPRPLLSRAQQRKRDGPDLAEYYYDAHL

SEQ ID NO: 41 - protein, 118 aa, Homo sapiens
  MISC_FEATURE (1)..(98): Human Neublasmin
MVLAMLGALHPRAGLSLFLHLILAVALLRSQPLRSQRSVPEAFSAPLELSQPLSGLVDDYGILPKHPRPRGPRPLLSRAQQRKRDGPDLAEYYYDAHLEGPRFEGKPIPNPLLGLDST

(19) United States
(12) Patent Application Publication
(10) Pub. No.: US 2008/0318853 A1
(43) Pub. Date: Dec. 25, 2008

(54) Diagnostic and Therapeutic Use of a Novel Growth Factor, Neublasmin

(75) Inventors: Mette Gronborg, Copenhagen (DK); Nikolaj Blom, Copenhagen (DK)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(73) Assignee: NSGENE A/S, Ballerup (DK)

(21) Appl. No.: 11/666,949

(22) PCT Filed: Nov. 4, 2005
(86) PCT No.: PCT/DK05/00707

§ 371 Date: May 2, 2007

Related U.S. Application Data
(60) Provisional application No. 60/625,173, filed on Nov. 5, 2004.

(30) Foreign Application Priority Data
Nov. 5, 2004 (DK) PA 2004 01710
Nov. 25, 2004 (EP) PCT/EP2004/053101

(51) Int. Cl. C07K 14/475; A61K 38/17; C12N 15/12; C12P 21/02; C07K 1/04; C07K 16/22; G01N 33/53; G01N 33/554; G01N 33/68; C12N 5/06; C12Q 1/68

(52) U.S. Cl. 514/12; 530/324; 536/23.1; 536/23.5; 435/69.1; 530/334; 530/387.9; 530/391.1; 530/387.3; 530/391.3; 530/391.7; 436/86; 436/501; 435/7.31; 435/7.2; 435/375; 435/7.92; 435/6

(57) ABSTRACT

The present invention relates to the field of diagnostic and therapeutic use of proteins and genes, in particular to the diagnostic and therapeutic use of a secreted human hormone/growth factor, Neublasmin, and use or the gene coding for Neublasmin in the diagnosis and treatment of testicular disorders, in particular diagnosis and treatment of germ cell tumours and infertility. The invention also relates to use of Neublasmin in the treatment of CNS disorders. Neublasmin is expressed at high levels in human adult testicles and in developing mouse testicles from pn 22 and onwards. Expression or Neublasmin is strongly up-regulated in carcinoma in situ. Expression is also seen in foetal and adult brain.

The present invention claims priority from Danish patent application DK PA 2004 01710 filed on 5 Nov. 2004. It claims the benefit of U.S. provisional application 60/625,173 filed on 5 Nov. 2004 and the benefit of International Patent Application No. PCT/EP2004/053101 filed on 25 Nov. 2004. All references cited in those applications and in the present application are incorporated by reference in their entirety.

### TECHNICAL FIELD

The present invention relates to the field of diagnostic and therapeutic use of proteins and genes, in particular to the diagnostic and therapeutic use of a secreted human hormone/growth factor, Neublasmin, and use of the gene coding for Neublasmin in the diagnosis and treatment of testicular disorders, in particular diagnosis and treatment of germ cell tumours and infertility, and in vitro uses as growth or trophic factors, for in vitro fertilisation, for stimulating sperm cells, or for insemination. The invention also relates to use of Neublasmin in the treatment of CNS disorders.

### BACKGROUND ART

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. There is a need to provide the protein effector as a therapeutic product. Administration of the effector to a subject in need thereof is useful in treatment of the pathological condition. Accordingly, there is a need for methods of treatment of a pathological condition brought on by a diminished or suppressed levels of the protein effector of interest. In addition, there is a need for methods of treatment of a pathological condition brought on by increased or up-regulated levels of the protein effector of interest. In a clinical setting a subject may be suspected of suffering from a condition brought on by altered or mis-regulated levels of a protein effector of interest. Therefore there is also a need to assay for the level of the protein effector of interest in a biological sample from such a subject, and to compare the level with that characteristic of a nonpathological condition.

Testicular cancer is the most common malignancy occurring in young adult men. In addition to its neoplastic and malignant features, this disorder also represents a developmental, endocrine and reproductive problem. Testicular cancer comprises a number of different diseases. The testis consists of several types of cells which form two main anatomical and functional compartments: the seminiferous tubules and the interstitial space. Nearly all of the main cell types in the testis can undergo neoplastic transformation, but germ cell-derived tumours constitute the vast majority of cases of testicular neoplasms (ENDOCRINOLOGY OF MALE REPRODUCTION, Robert McLachlan—Editor; TESTICULAR CANCER PATHOGENESIS, DIAGNOSIS AND ENDOCRINE ASPECTS; Chapter 13—Niels E. Skakkebaek, Ewa Rajpert-De Meyts and Jorma Toppari. Nov. 25, 2003; http://www.endotext.org/male/male13/maleframe13.htm).

Testicular tumours derived from germ cells are by far the most frequent neoplasms of the testis and comprise approximately 90-95% of cases. They are unique in comparison with other solid tumours for several reasons. Most of them occur in young adults. These tumours originate early in life and have a common preinvasive precursor, carcinoma in situ (CIS) which transforms further into overt tumours in young adulthood. Germ cell tumours have very high propensity to apoptosis and are extremely radio- and chemosensitive. As the germ cell tumours can be cured, an early diagnosis is of great clinical importance. About half of them can differentiate and form histologically variable forms. Epidemiology of testicular germ cell cancer has attracted growing attention, because the incidence has been steadily rising in recent decades (ENDOCRINOLOGY OF MALE REPRODUCTION, Robert McLachlan—Editor; TESTICULAR CANCER PATHOGENESIS, DIAGNOSIS AND ENDOCRINE ASPECTS; Chapter 13—Niels E. Skakkebaek, Ewa Rajpert-De Meyts and Jorma Toppari. Nov. 25, 2003; http://www.endotext.org/male/male13/maleframe13.htm).

The most common germ cell tumours of the young adults may be divided into preinvasive CIS, seminoma, nonseminoma and combined tumors. Morphology of CIS cells resemble closely that of immature germ cells (gonocytes). CIS cells are located inside seminiferous tubules, most frequently in a single row along the basement membrane. Seminoma cells are morphologically very close to CIS cells and proliferate as a homogeneous tumour, which retains features of germinal lineage. Nonseminomatous tumors display a variety of histological forms and differentiate along an embryonic lineage (embryonal carcinoma, teratoma, teratocarcinoma) or extra-embryonic tissue components (yolk sac tumor and choriocarcinoma). Teratomas are sometimes associated with rarely observed carcinoid tumors, which may be associated with carcinoid syndrome. The combined tumors contain elements of seminoma and nonseminomatous tumours but clinically are treated as nonseminoma. Both seminoma and nonseminoma originate from CIS and are mainly observed in young adult men. In other age groups, germ cell tumors are rare (ENDOCRINOLOGY OF MALE REPRODUCTION, Robert McLachlan—Editor; TESTICULAR CANCER PATHOGENESIS, DIAGNOSIS AND ENDOCRINE ASPECTS; Chapter 13—Niels E. Skakkebaek, Ewa Rajpert-De Meyts and Jorma Toppari. Nov. 25, 2003; http://www.endotext.org/male/male13/maleframe13. htm).

At present, diagnosis of testicular neoplasia at the preinvasive stage of CIS, which is asymptomatic, is only sporadic. Surgical testicular biopsy is currently the only sure diagnostic procedure for CIS. Diagnosis using scrotal Ultrasonography has been increasingly popular for assessment of the testicles. However, ultrasonic microlithiasis is not always confirmed histologically. In the vast majority of cases CIS progresses to overt tumors unnoticed. Late stage germ cell tumors secrete protein products that can be detected in circulating blood. These biochemical serum tumor markers are very helpful in diagnosis and monitoring of these tumors. Examples of these serum tumour markers include chorionic gonadotropin (HCG), alpha-fetoprotein (AFP), and lactate dehydrogenase (LDH). The most important in clinical practice are HCG and AFP, since they are very sensitive markers for nonseminomatous tumours, which in many cases have a more malignant clinical course than seminoma. In early stages of germ stage tumours, such as preinvasive CIS and in most cases of pure classical seminoma, none of the above mentioned markers are detectable in serum. Such early serum markers would be of significant clinical importance. Other markers, developed for immunohistochemical diagnosis of CIS cells and tumors in tissue sections, such as Placenta-like alkaline phosphatase (PLAP) and TRA-1-60, have been adapted for use as serum assays and begun to be used in clinical practice, although with mixed results (ENDOCRINOLOGY OF MALE REPRODUCTION, Robert McLachlan—Editor; TESTICULAR CANCER PATHOGENESIS, DIAGNOSIS AND ENDOCRINE ASPECTS; Chapter 13—Niels E. Skakkebaek, Ewa Rajpert-De Meyts and Jorma Toppari. Nov. 25, 2003; http://www.endotext.org/male/male13/maleframe13.htm).

Inter alia with the purpose of identifying markers for early diagnosis of germ cell neoplasms at the stage of CIS, possibly by a non-invasive method, Hoei-Hansen et al (Hoei-Hansen et al, Identification of genes differentially expressed in testes containing carcinoma in situ, Mol Hum Reprod, 10:423-431, 2004) have identified a number of known and uncharacterised genes differentially expressed in testes containing carcinoma in situ, Using cDNA microarray analysis, 895 genes that are expressed at significantly greater levels in human embyronal stem cells (ES) and embryonal carcinoma cell lines than in control samples have been identified. These 895 genes are candidates for involvement in the maintenance of a pluripotent undifferentiated phenotype (Spreger et al; “Gene expression patterns in human embryonic stem cells and human pluripotent germ cell tumours”, PNAS, 100:13350-13355, 2003).

Reduced male fertility is an increasing problem in the industrialised world. There is a need for understanding the mechanisms underlying the development and maturation of spermatids and spermatozoa and in particular for identifying cell signalling molecules involved in these events. Cell signalling molecules such as hormones and growth factors have the advantage that they can be supplied to an animal and remedy or rescue an aberrant signalling pathway. Cell signalling molecules involved in the development of spermatids and spermatozoa have potential as therapeutics for treatment of infertility and reduced fertility as well as potential for use in in vitro fertilisation.

It is one object of the present invention to provide a secreted protein for use in diagnosis of testicle cancer and for use in developing treatment of testicle cancer. It is a further object of the invention to provide a function to one of the numerous putative human genes which are annotated in Genbank as coding for a hypothetical protein.

None of cited references have succeeded in identifying Neublasmin, to which the present invention relates as an early marker for germ cell tumours. Neublasmin has the dual properties of being a good marker for preinvasive carcinoma in situ and being a secreted protein, which can be assayed in blood or sperm samples.

### SUMMARY OF THE INVENTION

The present invention relates to a polypeptide and gene called Neublasmin. The gene coding for Neublasmin is expressed at high levels in testicles. Expression of Neublasmin in mice starts at approximately day 22 after birth, i.e. at the same time as spermatocytes begin to undergo meiosis and differentiate into haploid round spermatoids. Neublasmin expression in testicles continues to be high into adulthood in both mouse and humans. Using immunohistochemistry the with anti-Neublasmin antibodies, it has been verified that Neublasmin protein found at high levels in spermatids and spermatozoa in adult human testes. The high increase in Neublasmin protein levels as spermatids mature, strongly indicates that Neublasmin peptides may have a function down-stream from the testicles, e.g. in epidydimis, spermatic duct, and/or during fertilisation.

The Neublasmin gene codes for a secreted growth factor with an N-terminal signal peptide. Secretion and proteolytical processing of Neublasmin has been verified by expressing a tagged human Neublasmin in mammalian cells and detecting the secreted peptides with antibodies against the tag and anti-Neublasmin antibodies.

The temporal expression pattern of Neublasmin in mouse testicles coincides with the expression of a testicular germ-cell protease, proconvertase-4, PC4 (Nakayama et al 1992, J Biol Chem 267:5897-5900). The mature polypeptide sequence of Neublasmin contains a Proconvertase-4 cleavage motif. The date provided herein show that Neublasmin protein is indeed subject to proteolytical processing after secretion. The proteolytical processing results in the formation of a C- and an N-terminal peptide. The present inventors therefore believe that one or both peptides resulting from this cleavage are bioactive. The temporal expression pattern and the fact that Neublasmin is a secreted growth factor strongly indicates that Neublasmin is involved in spermagogenesis and that reduced Neublasmin expression or processing may be one of the components of reduced male fertility. This is further supported by the fact that a PC4 null mice have strongly reduced fertility (Mbikay et al 1997, PNAS 94:6842-6846), which indicates that aberrant processing of Neublasmin may be a cause of this reduced fertility.

As evidenced by the appended examples, the present inventors have also determined that Neublasmin expression is strongly and specifically up-regulated in carcinoma in situ (CIS), which may lead to various forms of testicle cancer, specifically seminoma and non-seminoma including embryonal carcinoma, teratoma, and teratocarcinoma. Neublasmin expression remains high in later stages of testicle cancer. As Neublasmin codes for a secreted protein, the up-regulation of Neublasmin expression in carcinoma in situ can be easily detected by a quantitative assessment of the level of Neublasmin polypeptide in a biological sample isolated from an animal suspected of being afflicted with CIS. Today, the only reliable way of diagnosing carcinoma in situ is through biopsy. As CIS normally goes unnoticed by the afflicted individual, biopsy is not a clinically relevant method of diagnosis. It should also be added that at the stage of carcinoma in situ, treatment is relatively easy through the use of orchidectomy, radiotherapy, chemotherapy, or surveillance. At later stages of cancer, treatment is more complicated and the success rate is much lower. By following the amount of Neublasmin polypeptide in a biological sample during treatment, the progress of said treatment can be followed.

In a first aspect the invention relates to a Neublasmin peptide selected from the group consisting of

i) a peptide having an amino acid sequence selected from the group consisting of SEQ ID No 26, 27, 28, 29, 30, 31 32, 33, 34, and 35;

ii) a bioactive peptide having at least 60% sequence identity to a peptide selected from the group consisting of SEQ ID No 26, 27, 28, 29, 30, 31 32, 33, 34, and 35;

iii) a bioactive fragment of at least 15 contiguous amino acids of a peptide selected from the group consisting of SEQ ID No 26, 27, 28, 29, 30, 31 32, 33, 34, and 35;

iv) a peptide obtainable by pro-convertase-4 cleavage of a polypeptide having an amino acid sequence selected from the group consisting of SEQ ID No. 9, 10, 14, 18, and 25; and

v) a mature polypeptide obtainable from the culture medium of a mammalian cell expressing a polynucleotide encoding a polypeptide having an amino acid sequence selected from the group consisting of SEQ ID No. 2, 4, 5, 7, 8, 12, 13, 16, 17, 23, and 24.

The Neublasmin peptides are all secreted peptides resulting from the subsequent proteolytical processing of Neublasmin. These Neublasmin peptides according to the present invention may have several utilities. First the peptides can be used for immunisation to raise antibodies against Neublasmin peptides for use in the diagnostic methods described above. Furthermore, the peptides may be used in a method of treatment of infertility caused by insufficient expression and/or processing of Neublasmin in the testicles. Furthermore, the peptides may be used in connection with in vitro fertilisation, for stimulating sperm cells in vitro, or for insemination as Neublasmin expression is evidently closely associated with maturation of spermatocytes. Neublasmin peptides may also be used in a method of identification of a Neublasmin peptide receptor and in the design of peptide analogues. Finally, the Neublasmin peptides may be used as growth factors in mammalian cell cultures, in particular in cultures comprising embryonal stem cells. This utility is based on the fact that the gene expression pattern in germ cell tumours and embryonal stem cell lines show many similarities (Sperger et al 2003, PNAS 100:13350-13355). A secreted polypeptide having an effect on spermatogenesis is therefore likely to have an effect on cell division and/or differentiation in vitro.

In a further aspect, the invention relates to a nucleic acid comprising a nucleic acid sequence coding for a Neublasmin peptide fused to a signal peptide coding sequence and to an expression vector comprising said nucleic acid. Such nucleic acids and expression vectors may be used for recombinant expression of Neublasmin peptides in mammalian cells, where a signal peptide is required for secretion. Preferably the signal peptide is a heterologous signal peptide.

The invention furthermore relates to a method of producing Neublasmin peptides of the invention comprising chemical synthesis using custom made peptides. The synthesis may comprise solid phase or solution phase chemical synthesis. Several of the Neublasmin peptides are so short that chemical synthesis is economically more feasible than recombinant production.

In a further aspect, the invention relates to a pharmaceutical composition comprising a Neublasmin peptide according to the invention and a pharmaceutically acceptable carrier. The invention also relates to use of a Neublasmin peptide according to the invention for the manufacture of a medicament. The therapeutic effect of Neublasmin may be mediated through an effect on growth, survival, and/or differentiation of targeted cells.

The invention in another aspect relates to an antibody generated against a Neublasmin peptide according to the invention. These antibodies may be used for analytical purposes, for diagnostic purposes or as therapeutic antibodies for treatment of conditions involving overproduction of Neublasmin. The antibodies may be generated against a C- or an N-terminal Neublasmin peptide. The peptides are preferably conjugated to a carrier protein prior to immunisation. The invention also relates to an immunoconjugate of such antibodies and a conjugate selected from the group consisting of: a cytotoxic agent such as a chemotherapeutic agent, a toxin, or a radioactive isotope; a member of a specific binding pair, such as avidin or streptavidin or an antigen; an enzyme capable of producing a detectable product.

In one aspect the invention relates to a method for determining the presence of or predisposition to a disease associated with altered levels of a Neublasmin peptide of the invention in a first mammalian subject, the method comprising:

a) measuring the level of expression of the polypeptide in an isolated biological sample from the first mammalian subject; and

b) comparing the amount of the polypeptide in the sample of step (a) to the amount of the polypeptide present in an isolated biological control sample from a second mammalian subject known not to have, or not to be predisposed to, the disease,

wherein an alteration in the expression level of the polypeptide in the first subject as compared to the control sample indicates the presence of or predisposition to the disease.

In a further aspect, the invention relates to a method of identifying an agent that binds to a Neublasmin peptide of the invention, the method comprising:

(a) introducing the polypeptide to the agent; and

(b) determining whether the agent binds to the polypeptide.

The agent may be cellular receptor or a downstream effector. The agent may be identified using yeast two hybrid screening.

In a further aspect, the invention relates to a method for determining the presence of or predisposition to a disease associated with altered levels of a secreted Neublasmin polypeptide in a first mammalian subject, the method comprising:

a) measuring the level of expression of the polypeptide in an isolated biological sample from the first mammalian subject; and

b) comparing the amount of the polypeptide in the sample of step (a) to the amount of the polypeptide present in an isolated biological control sample from a second mammalian subject known not to have, or not to be predisposed to, the disease,

wherein an alteration in the expression level of the polypeptide in the first subject as compared to the control sample indicates the presence of or predisposition to the disease,
wherein the disease selected from the group consisting of male infertility, carcinoma in situ, seminoma and non-seminoma including embryonal carcinoma, teratoma, and teratocarcinoma.

In a similar aspect the determination may be performed by measuring altered levels of a Neublasmin nucleic acid.

The polypeptide for this aspect may be selected from the group consisting of

a) a mature form of the amino acid sequence selected from the group consisting of SEQ ID No. 9, 10, 14, 18, and 25;

b) a variant of a mature form of the amino acid sequence selected from the group consisting of SEQ ID No. 9, 10, 14, 18, and 25, wherein the variant has at least 50% sequence identity to said SEQ ID No.;

c) the amino acid sequence selected from the group consisting of SEQ ID No. 2, 4, 5, 7, 8, 12, 13, 16, 17, 23, and 24;

d) a variant of the amino acid sequence selected from the group consisting of SEQ ID No. 2, 4, 5, 7, 8, 12, 13, 16, 17, 23, and 24, wherein the variant has at least 50% sequence identity to said SEQ ID No.;

e) a mature polypeptide obtainable from the culture medium of a mammalian cell expressing a polynucleotide encoding a polypeptide having an amino acid sequence selected from the group consisting of SEQ ID No. 2, 4, 5, 7, 8, 12, 13, 16, 17, 23, and 24; and

f) a fragment of at least 20 contiguous amino acids of any of a) through d).

More preferably, the polypeptide is selected from the group consisting of polypeptides having the amino acid sequence of SEQ ID NO 2, 4, 5, 7, 8, 9, 10, and fragments of at least 20 contiguous amino acid of any of these, SEQ ID NO 26, 27, 28, and 29 and fragments of at least 15 contiguous amino acids of any of these.

Preferably, the isolated biological sample is selected from the group consisting of cells, tissue, blood, blood serum, blood plasma, lymph, and sperm. More preferably the isolated biological sample is sperm, blood, blood serum, or blood plasma.

The disease may be carcinoma in situ, seminoma and non-seminoma including embryonal carcinoma, teratoma, and teratocarcinoma. Preferably the disease is carcinoma in situ.

In another embodiment, the disease is male infertility.

In one embodiment, the secreted polypeptide is measured using an antibody. Preferably, the antibody is directed against a peptide having the amino acid sequence of SEQ ID NO 36, 37, or 38.

In one aspect, the invention relates to use of a neublasmin polypeptide for the preparation of a medicament for the treatment of male infertility.

The neublasmin polypeptide for this use is preferably selected from the group consisting of polypeptides having the amino acid sequence of SEQ ID NO 2, 4, 5, 7, 8, 9, 10, and fragments of at least 20 contiguous amino acid of any of these, SEQ ID NO 26, 27, 28, and 29 and fragments of at least 15 contiguous amino acids of any of these.

In a still further embodiment, the invention relates to use of a neublasmin polypeptide as a growth factor in mammalian cell culture in vitro. This use may comprise stimulating sperm cells in vitro, use for in vitro fertilisation, or for insemination.

The invention also relates to a method of treatment of male infertility comprising administering to an individual in need thereof a therapeutically effective amount of a neublasmin polypeptide.

In a further aspect, the invention relates to an ELISA kit comprising

i) a first antibody capable of forming a species specific linkage to a first Neublasmin epitope,

ii) a surface for immobilisation of antibody or Neublasmin sample;

iii) a detectable label capable of being linked to a Neublasmin polypeptide through a species specific linkage; and

iv) buffers and reagents.

A species specific linkage includes the linkage formed between antibody and antigen, the linkage formed between biotin and avidin and other similar linkages which are typically used for linking a detectable label (e.g. an enzyme) to an antigen on a protein.

In one embodiment, the detectable label is capable of being linked to a second Neublasmin epitope through at least one species specific linkage, said at least one species specific linkage comprising a second antibody capable of binding a second Neublasmin epitope.

In another embodiment, the detectable label is capable of being linked to the first antibody through at least one species specific linkage.

In a further aspect, the invention relates to a kit for detection of a Neublasmin polypeptide, comprising

i) an antibody capable of binding to a Neublasmin epitope,

ii) a surface for immobilisation of antibody or Neublasmin polypeptide,

iii) a labelled Neublasmin polypeptide comprising said Neublasmin epitope, and

iv) buffers and reagents.

In one embodiment, the labelled Neublasmin polypeptide is radioactively labelled, thereby forming a RIA kit.

In another embodiment, the labelled Neublasmin polypeptide is covalently linked to an enzyme, thereby forming a competitive ELISA kit.

In a further embodiment, the invention relates to a kit for detection of a Neublasmin mRNA comprising either a labelled nucleic acid probe capable of hybridising in situ with a Neublasmin mRNA, or a pair of Neublasmin primers for quantitative amplification of a Neublasmin cDNA.

### BRIEF DESCRIPTION OF THE DRAWINGS

FIG. 1: Clustal W (1.82) multiple sequence alignment of human, mouse and rat Neublasmin.

FIG. 1a: full length proteins. The predicted signal peptide is shown in grey. A putative furin propeptide cleavage site is underlined in the human and rat sequence. FIG. 1b: mature proteins. The proprotein-convertase-4 cleavage site is underlined.
* indicates positions which have a single, fully conserved residue.
: indicates that one of the following ‘strong’ groups is fully conserved: -STA, NEQK, NHQK, NDEQ, QHRK, MILV, MILF, HY, FYW.
. indicates that one of the following ‘weaker’ groups is fully conserved: -CSA, ATV, SAG, STNK, STPA, SGND, SNDEQK, NDEQHK, NEQHRK, VLIM, HFY.

FIG. 2: RT-PCR using primers 891 and 892. For details, see Example 5. The arrow points to a band of 210 bp amplified from human testis cDNA.

FIG. 3a: SignalP NN plot of human Neublasmin. FIG. 3b: SignalP HMM plot of human Neublasmin.

FIG. 4a: SignalP NN plot of mouse Neublasmin. FIG. 4b: SignalP HMM plot of mouse Neublasmin.

FIG. 5a: SignalP NN plot of rat Neublasmin. FIG. 5b: SignalP HMM plot of rat Neublasmin.

FIG. 6a: SignalP NN plot of human (N-4) Neublasmin. FIG. 6b: SignalP HMM plot of human (N-4) Neublasmin.

FIG. 7. Expression of Neublasmin in human tissues analyzed by quantitative RT-PCR as described in Example 8. FIG. 7A shows the relative expression of human Neublasmin normalised to β₂-microglobulin (relative to tissue with the lowest normalized expression). Results should be interpreted with caution as β₂-microglobulin expression levels vary between some tissues. Note that the scale is a log scale. FIG. 7B shows the non-normalised relative expression of human Neublasmin (relative to tissue with the lowest expression). In FIG. 7B the scale is not a log-scale.

FIG. 8: Neublasmin expression was analyzed during mouse spermatogenesis. Testes were sampled from postnatal development as indicated in FIG. 8A. RNA was purified and cDNA prepared. mNeublasmin expression was analyzed by RT-PCR using primers [ns-gene-1 and ns-gene-4] amplifying a fragment of 254 bp from mouse Neublasmin cDNA. Expression analyzed using the same primers and RT-PCR in normal human testis and in CIS, (carcinoma in situ) is also shown (the expected product amplified from human cDNA is 251 bp). As seen in FIG. 8A a product of the correct size (arrow) is amplified from mouse testis cDNA sampled at pn 22-52 whereas only larger (unspecific) products are amplified from younger ages. Expression is first seen at pn22 (arrow). Also a specific band of the expected size could be amplified from both cDNAs derived from human material included in the analysis. The amplified band has been sequenced confirming that it corresponds to Neublasmin cDNA.

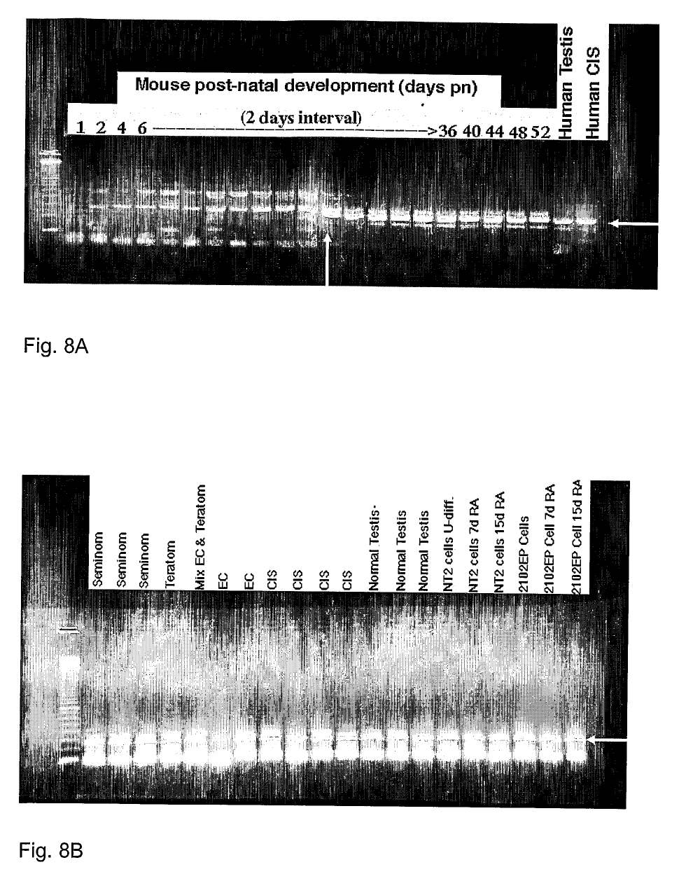

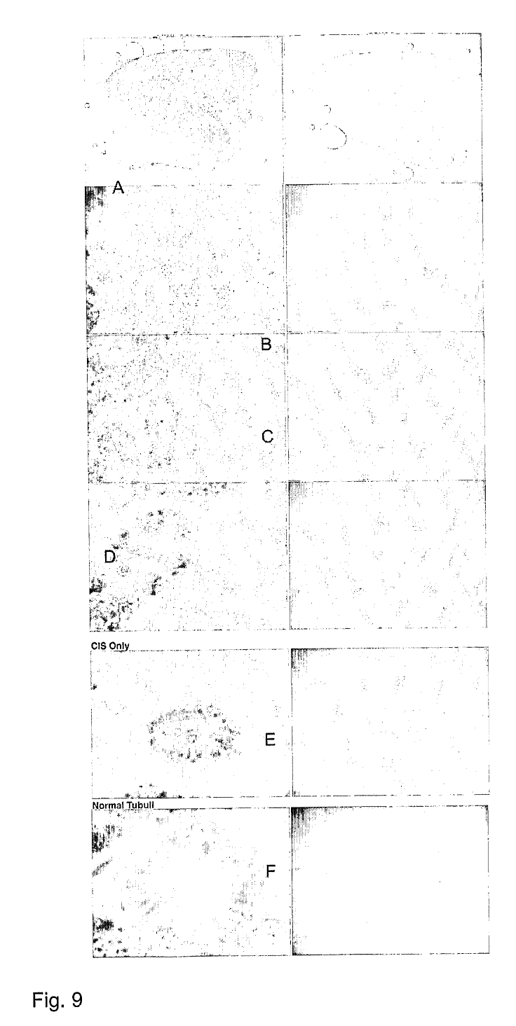

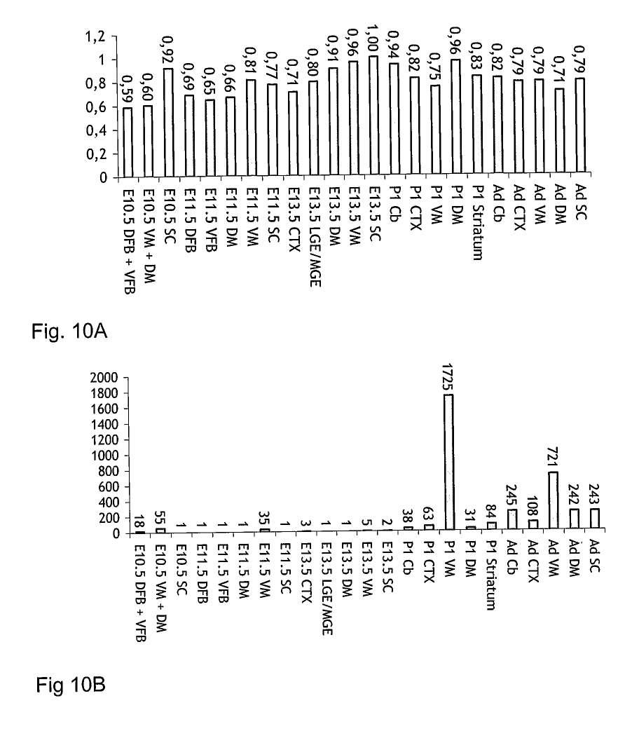

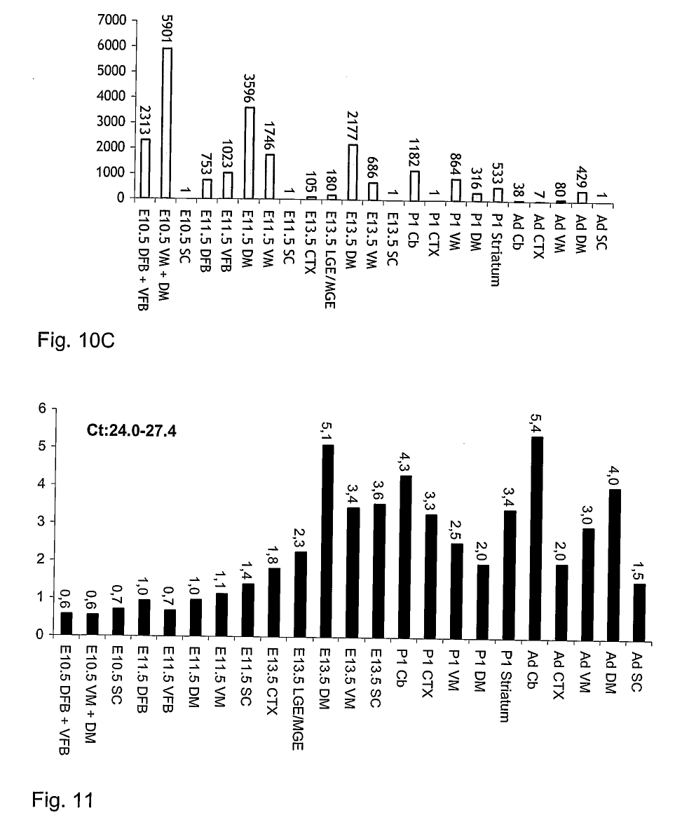

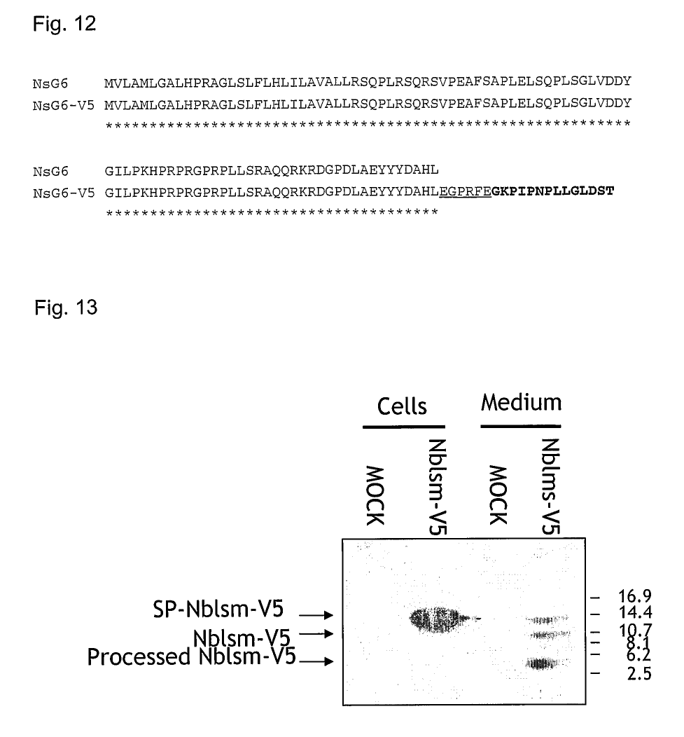

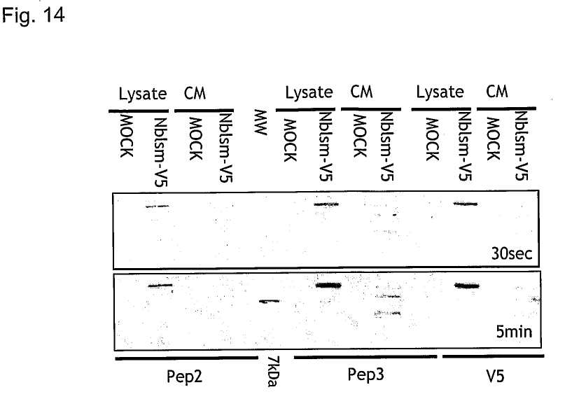

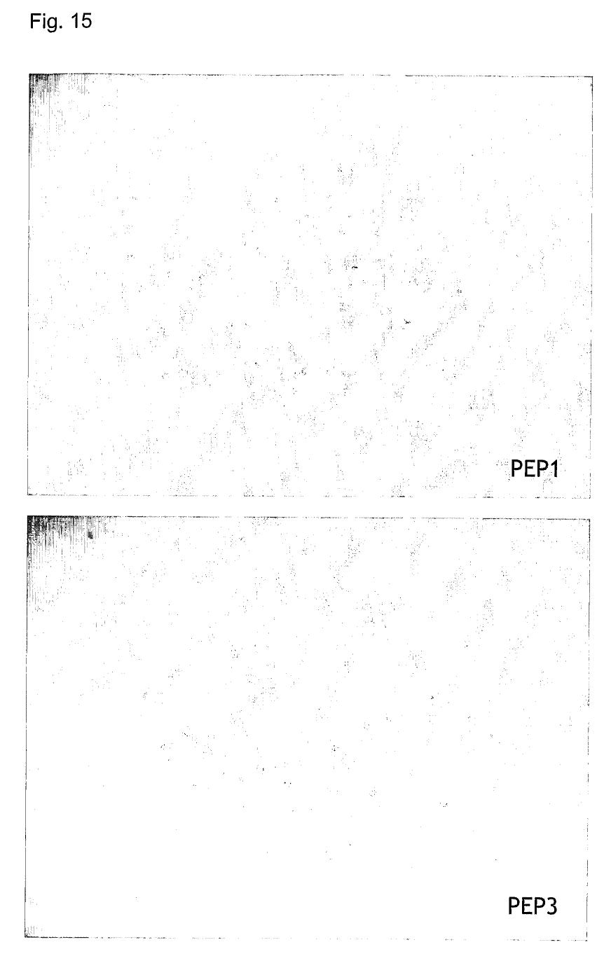

In FIG. 8B, RT-PCR analysis was carried out as described for the analysis shown in FIG. 8A except that cDNAs (all of human origin) derived from a number of different testicular tumours (Seminoma, teratoma and EC=Embryonal Carcinoma) and two cell lines derived (NT2 and 2102EP) were included in the analysis. Samples derived from NT2 and 2102EP cells treated with retinoic acid (═RA) for 7-15 days (d) were also included in addition to samples derived from the undifferentiated (u-diff) cultures. NT2 and 2102EP are cloned cell lines both derived from EC. NT2 cells are capable of differentiating after exposure to RA whereas 2102Ep cells are not (Mavilio et al., 1988. Differentiation. 37:73-9). As seen in FIG. 8B, Neublasmin of the expected size (arrow) is expressed in all tissues and cells tested except one sample derived from an EC. The band has been sequenced and the identity to Neublasmin has been confirmed.

FIG. 9: shows in situ hybridisation on a testicular biopsy carried out as described in Example 9. Pictures in the left column shows sections hybridised with a Neublasmin anti-sense probe. Pictures in the right column show the corresponding sections hybridised with a Neublasmin sense probe (control). A-D show different magnifications of tissue with carcinoma in situ mixed with normal testis tissue. E shows carcinoma in situ only. F shows normal tubuli.

FIG. 10: GAPDH (A), ALDH1A1 (B) and OTX2 (C) expression in developing mouse CNS.

FIG. 11: Neublasmin expression in developing mouse CNS. The expression level is normalised relative to the expression of GAPDH. Tissue with the lowest relative expression is set to 1.

FIG. 12: Alignment of the Neublasmin protein sequence and the Neublasmin-V5 fusion protein sequence. The spacer region is underlined and the V5 tag is shown in bold.

FIG. 13: Anti V5 western blot of cell lysates and conditioned medium from mock and pHsC.hNeublasmin-V5.W transfected HEK293 cells.

FIG. 14: Detection of Neublasmin-V5 protein in western blotting using PEP2/PEP3 serum as well as anti V5 antibody.

FIG. 15: Immunohistochemistry on testis sections using purified PEP1 and PEP3 antibody.

### DEFINITIONS

Neublasmin, as used herein, refers to the amino acid sequences of substantially purified Neublasmin obtained from any species, particularly mammalian, including chimpanzee, bovine, ovine, porcine, murine, equine, and preferably human, from any source whether natural, synthetic, semi-synthetic, or recombinant. The term Neublasmin includes pre-Neublasmin, mature Neublasmin and C- and N-terminal peptides.

The term “agonist”, as used herein, refers to a molecule which, when bound to Neublasmin, increases or prolongs the duration of the effect of Neublasmin. Preferably the agonist has this effect when not bound to Neublasmin. Agonists may include proteins, nucleic acids, carbohydrates, or any other molecules which bind to and modulate the effect of Neublasmin.

An “allele” or “allelic sequence”, as used herein, is an alternative form of the gene encoding Neublasmin. Alleles may result from at least one mutation in the nucleic acid sequence and may result in altered mRNAs or polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Altered” nucleic acid sequences encoding Neublasmin, as used herein, include those with deletions, insertions, or substitutions of different nucleotides resulting in a polynucleotide that encodes the same or a functionally equivalent Neublasmin. Included within this definition are polymorphisms which may or may not be readily detectable using a particular oligonucleotide probe of the polynucleotide encoding Neublasmin, and improper or unexpected hybridization to alleles, with a locus other than the normal chromosomal locus for the polynucleotide sequence encoding Neublasmin. The encoded protein may also be “altered” and contain deletions, insertions, or substitutions of amino acid residues which produce a silent change and result in a functionally equivalent Neublasmin. Deliberate amino acid substitutions may be made on the basis of similarity in polarity, charge, solubility, hydrophobicity, hydrophilicity, and/or the amphipathic nature of the residues as long as the biological and/or immunological activity of Neublasmin is retained.

“Amino acid sequence”, as used herein, refers to an oligopeptide, peptide, polypeptide, or protein sequence, and fragment thereof, and to naturally occurring or synthetic molecules. Fragments of Neublasmin are preferably about 5 to about 15 amino acids in length and retain the biological activity or the immunological activity of Neublasmin. Where “amino acid sequence” is recited herein to refer to an amino acid sequence of a naturally occurring protein molecule, amino acid sequence, and like terms, are not meant to limit the amino acid sequence to the complete, native amino acid sequence associated with the recited protein molecule.

“Amplification”, as used herein, refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.).

The term “antagonist”, as used herein, refers to a molecule which, when bound to Neublasmin, decreases the amount or the duration of the effect of the biological or immunological activity of Neublasmin. In other embodiments, the antagonist has this effect when not bound to Neublasmin. Antagonists may include proteins, nucleic acids, carbohydrates, antibodies or any other molecules which decrease the effect of Neublasmin.

As used herein, the term “antibody” refers to intact molecules as well as fragments thereof, such as Fab, F(ab′), and Fv, which are capable of binding the epitopic determinant. Antibodies that bind Neublasmin polypeptides can be prepared using intact polypeptides or fragments containing small peptides of interest as the immunizing antigen. The polypeptide or oligopeptide used to immunize an animal can be derived from the translation of RNA or synthesized chemically and can be conjugated to a carrier protein, if desired. Commonly used carriers that are chemically coupled to peptides include bovine serum albumin and thyroglobulin, keyhole limpet hemocyanin. The coupled peptide is then used to immunize the animal (e.g., a mouse, a rat, or a rabbit).

The term “antigenic determinant”, as used herein, refers to that fragment of a molecule (i.e., an epitope) that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The term “antisense”, as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The term “biologically active”, as used herein, refers to a protein having structural, regulatory, or biochemical functions of a naturally occurring Neublasmin. Likewise, “immunologically active” refers to the capability of the natural, recombinant, or synthetic Neublasmin, or any oligopeptide thereof, to induce a specific immune response in appropriate animals or cells and to bind with specific antibodies.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of PNA or LNA molecules.

A “composition comprising a given polynucleotide sequence”, as used herein, refers broadly to any composition containing the given polynucleotide sequence. The composition may comprise a dry formulation or an aqueous solution. Compositions comprising polynucleotide sequences encoding Neublasmin or fragments thereof may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS) and other components (e.g., Denhardt's solution, dry milk, salmon sperm DNA, etc.).

A “deletion”, as used herein, refers to a change in the amino acid or nucleotide sequence and results in the absence of one or more amino acid residues or nucleotides.

The term “derivative”, as used herein, refers to the chemical modification of a nucleic acid encoding or complementary to Neublasmin or the encoded Neublasmin. Such modifications include, for example, replacement of hydrogen by an alkyl, acyl, or amino group. A nucleic acid derivative encodes a polypeptide which retains the biological or immunological function of the natural molecule. A derivative polypeptide is one which is modified by glycosylation, pegylation, or any similar process which retains the biological or immunological function of the polypeptide from which it was derived.

The term “homology”, as used herein, refers to a degree of complementarity. There may be partial homology or complete homology (i.e., identity). A partially complementary sequence that at least partially inhibits an identical sequence from hybridizing to a target nucleic acid is referred to using the functional term “substantially homologous.” The inhibition of hybridization of the completely complementary sequence to the target sequence may be examined using a hybridization assay (Southern or northern blot, solution hybridization and the like) under conditions of low stringency. A substantially homologous sequence or hybridization probe will compete for and inhibit the binding of a completely homologous sequence to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity). In the absence of non-specific binding, the probe will not hybridize to the second non-complementary target sequence.

The term “humanized antibody”, as used herein, refers to antibody molecules in which amino acids have been replaced in the non-antigen binding regions in order to more closely resemble a human antibody, while still retaining the original binding ability.

The term “hybridization”, as used herein, refers to any process by which a strand of nucleic acid binds with a complementary strand through base pairing.

The term “hybridization complex”, as used herein, refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution or between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

An “insertion” or “addition”, as used herein, refers to a change in an amino acid or nucleotide sequence resulting in the addition of one or more amino acid residues or nucleotides, respectively, as compared to the naturally occurring molecule.

“Microarray” refers to an array of distinct polynucleotides or oligonucleotides synthesized on a substrate, such as paper, nylon or other type of membrane, filter, chip, glass slide, or any other suitable solid support.

The term “modulate”, as used herein, refers to a change in the activity of Neublasmin. For example, modulation may cause an increase or a decrease in protein activity, binding characteristics, or any other biological, functional or immunological properties of Neublasmin.

“Nucleic acid sequence”, as used herein, refers to an oligonucleotide, nucleotide, or polynucleotide, and fragments thereof, and to DNA or RNA of genomic or synthetic origin which may be single- or double-stranded, and represent the sense or antisense strand. “Fragments” are those nucleic acid sequences which are greater than 60 nucleotides than in length, and most preferably includes fragments that are at least 100 nucleotides.

The term “oligonucleotide” refers to a nucleic acid sequence of at least about 6 nucleotides to about 60 nucleotides, preferably about 15 to 30 nucleotides, and more preferably about 20 to 25 nucleotides, which can be used in PCR amplification or a hybridization assay, or a microarray. As used herein, oligonucleotide is substantially equivalent to the terms “amplimers”, “primers”, “oligomers”, and “probes”, as commonly defined in the art.

“Peptide nucleic acid”, PNA as used herein, refers to an antisense molecule or anti-gene agent which comprises an oligonucleotide of at least five nucleotides in length linked to a peptide backbone of amino acid residues which ends in lysine. The terminal lysine confers solubility to the composition. PNAs may be pegylated to extend their lifespan in the cell where they preferentially bind complementary single stranded DNA and RNA and stop transcript elongation (Nielsen, P. E. et al. (1993) Anticancer Drug Des. 8:53-63).

The term “sample”, as used herein, is used in its broadest sense. A biological sample suspected of containing nucleic acid encoding Neublasmin, or fragments thereof, or Neublasmin itself may comprise a bodily fluid, extract from a cell, chromosome, organelle, or membrane isolated from a cell, a cell, genomic DNA, RNA, or cDNA (in solution or bound to a solid support, a tissue, a tissue print, and the like).

The terms “specific binding” or “specifically binding”, as used herein, refers to that interaction between a protein or peptide and an agonist, an antibody and an antagonist. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions. One or more factors be may be varied to generate conditions of either low or high stringency different from, but equivalent to, the above listed conditions.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

A “substitution”, as used herein, refers to the replacement of one or more amino acids or nucleotides by different amino acids or nucleotides, respectively.

“Transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and particle bombardment. Such “transformed” cells include stably transformed cells in which the inserted DNA is capable of replication either as an autonomously replicating plasmid or as part of the host chromosome. They also include cells, which transiently express the inserted DNA or RNA for limited periods of time.

A “variant” of Neublasmin, as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both.

“Sequence Identity”:

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTP programs of Altschul, et al. (1990) J. Mol. Biol. 215:403-410.

In order to characterize the identity, subject polypeptide sequences are aligned so that the highest order homology (match) is obtained. Based on these general principles the “percent identity” of two amino acid sequences may be determined using the BLASTP algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Matrix=Blosum62; Open gap=11; Extension gap=1; Penalties gap x_dropoff=50; Expect=10; Word size=3; Filter on). The BLAST algorithm performs a two-step operation by first aligning two sequences based on the settings and then determining the % sequence identity in a range of overlap between two aligned sequences. In addition to % sequence identity, BLASTP also determines the % sequence similarity based on the settings.

In order to characterize the identity, subject polynucleotide sequences are aligned so that the highest order homology (match) is obtained. Based on these general principles, the “percent identity” of two nucleic acid sequences may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Reward for a match=1; Penalty for a mismatch=−2; Strand option=both strands; Open gap=5; Extension gap=2; Penalties gap x_dropoff=50; Expect=10; Word size=11; Filter on). The BLASTN algorithm determines the % sequence identity in a range of overlap between two aligned nucleotide sequences. For the purposes of the present invention the percent sequence identity is preferably calculated in a range of overlap of at least 100 nucleotides, the range being determined by BLASTN under default settings. More preferably the range of overlap is at least 300 nucleotides.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. More preferably gap opening/gap extension penalties of −12/−2 are used. The ALIGN program (version 2.0) is also part of the FASTA sequence alignment software package (Pearson W R, Methods Mol Biol, 2000, 132:185-219). Align calculates sequence identities based on a global alignment. Align0 does not penalise to gaps in the end of the sequences. When utilizing the ALIGN og Align0 program for comparing amino acid sequences, a BLOSUM50 substitution matrix with gap opening/extension penalties of −12/−2 is preferably used.

Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference. The homology between two protein sequences may suitably be determined by means of computer programs known in the art such as GAP provided in the GCG program package [Needleman, S. B. and Wunsch, C. D., Journal of Molecular Bioloqy, 1970 48 443-453]. Using GAP with the following settings for polypeptide sequence comparison: GAP creation penalty of 8 and GAP extension penalty of 2. The DNA sequence homology may suitably be determined by means of computer programs known in the art, such as GAP provided in the GCG program package [Needleman, S. B. and Wunsch C. D., Journal of Molecular Biology 1970 48 443-453]. Using GAP with the following settings for DNA sequence comparison: GAP creation penalty of 50 and GAP extension penalty of 3.

A preferred algorithm for identifying further sequences with homology to Neublasmin is a Blast, because it uses local alignment. For the comparison of sequence identity between two sequences of the same or substantially the same length (pre-protein vs. preprotein, mature protein vs. mature protein, peptide vs. peptide), a full length alignment algorithm, such as ALIGN is preferred.

### DETAILED DESCRIPTION

The present invention relates to a polypeptide and gene called Neublasmin. The gene coding for Neublasmin is expressed at high levels in testicles. Expression of Neublasmin in mice starts at approximately day 22 after birth, i.e. at the same time as spermatocytes begin to undergo meiosis and differentiate into haploid round spermatoids. Neublasmin expression in testicles continues to be high into adulthood in both mouse and humans. Using immunohistochemistry the with anti-Neublasmin antibodies, it has been verified that Neublasmin protein found at high levels in spermatids and spermatozoa in adult human testes. The high increase in Neublasmin protein levels as spermatids mature, strongly indicates that Neublasmin peptides may have a function down-stream from the testicles, e.g. in epidydimis, spermatic duct, and/or during fertilisation. The Neublasmin gene codes for a secreted growth factor with an N-terminal signal peptide. Secretion and proteolytical processing of Neublasmin has been verified by expressing a tagged human Neublasmin in mammalian cells and detecting the secreted peptides with antibodies against the tag and anti-Neublasmin antibodies. The temporal expression pattern of Neublasmin in mouse testicles coincides with the expression of a testicular germ-cell protease, proconvertase-4, PC4 (Nakayama et al 1992, J Biol Chem 267:5897-5900). The mature polypeptide sequence of Neublasmin contains a Proconvertase-4 cleavage motif. The date provided herein show that Neublasmin protein is indeed subject to proteolytical processing after secretion. The proteolytical processing results in the formation of a C- and an N-terminal peptide. The present inventors therefore believe that one or both peptides resulting from this cleavage are bioactive. The temporal expression pattern and the fact that Neublasmin is a secreted growth factor strongly indicates that Neublasmin is involved in spermagogenesis and that reduced Neublasmin expression or processing may be one of the components of reduced male fertility. This is further supported by the fact that a PC4 null mice have strongly reduced fertility (Mbikay et al 1997, PNAS 94:6842-6846), which indicates that aberrant processing of Neublasmin may be a cause of this reduced fertility.

As evidenced by the appended examples, the present inventors have also determined that Neublasmin expression is strongly and specifically up-regulated in carcinoma in situ (CIS), which may lead to various forms of testicle cancer, specifically seminoma and non-seminoma including embryonal carcinoma, teratoma, and teratocarcinoma. Neublasmin expression remains high in later stages of testicle cancer. As Neublasmin codes for a secreted protein, the up-regulation of Neublasmin expression in carcinoma in situ can be easily detected by a quantitative assessment of the level of Neublasmin polypeptide in a biological sample isolated from an animal suspected of being afflicted with CIS. Today, the only reliable way of diagnosing carcinoma in situ is through biopsy. As CIS normally goes unnoticed by the afflicted individual, biopsy is not a clinically relevant method of diagnosis. It should also be added that at the stage of carcinoma in situ, treatment is relatively easy through the use of orchidectomy, radiotherapy, chemotherapy, or surveillance. At later stages of cancer, treatment is more complicated and the success rate is much lower. By following the amount of Neublasmin polypeptide in a biological sample during treatment, the progress of said treatment can be followed.

Of course, carcinoma in situ may also be diagnosed using a Neublasmin probe (in situ hybridisation) or using quantitative PCR on a biopsy from a patient as it the present inventors have shown that the level of Neublasmin mRNA is upregulated in carcinoma in situ. As carcinoma in situ only constitutes a small fraction of the cells of a biopsy, the upregulation in some cases may go unnoticed in quantitative PCR, but will be detectable with in situ hybridisation.

The Neublasmin peptides are all secreted peptides resulting from the subsequent proteolytical processing of Neublasmin. These Neublasmin peptides according to the present invention may have several utilities. First the peptides can be used for immunisation to raise antibodies against Neublasmin peptides for use in the diagnostic methods described above. Furthermore, the peptides may be used in a method of treatment of infertility caused by insufficient expression and/or processing of Neublasmin in the testicles. Furthermore, the peptides may be used in connection with in vitro fertilisation, for stimulating sperm cells in vitro, or for insemination as Neublasmin expression is evidently closely associated with maturation of spermatocytes. Neublasmin peptides may also be used in a method of identification of a Neublasmin peptide receptor and in the design of peptide analogues. Finally, the Neublasmin peptides may be used as growth factors in mammalian cell cultures, in particular in cultures comprising embryonal stem cells. This utility is based on the fact that the gene expression pattern in germ cell tumours and embryonal stem cell lines show many similarities (Sperger et al 2003, PNAS 100:13350-13355). A secreted polypeptide having an effect on spermatogenesis is therefore likely to have an effect on cell division and/or differentiation in vitro.

It has recently been shown that mouse spermatogonial stem cells can be expanded in vitro and be used for transplantation and restoration of fertility (Hiroshi Kubota, Mary R. Avarbock, and Ralph L. Brinster, “Growth factors essential for self-renewal and expansion of mouse spermatogonial stem cells”, PNAS published Nov. 1, 2004, 10.1073/pnas.0407063101). Neublasmin is expressed in male germ cells and is therefore likely to have an effect on spermatogonial stem cells.

The invention relates to the use of a novel protein, identified as Neublasmin. The protein has been identified in human beings (SEQ ID No. 2, 4, 5, 6, 7, 8, 9, and 10), mouse (SEQ ID No. 12, 13, and 14), rat (SEQ ID No. 16, 17, and 18), and Chimpanzee (SEQ ID No 23, 24, and 25)

Human Neublasmin is a 98 amino acid secreted hormone or growth factor protein expressed at high levels in testis and brain. Human Neublasmin is 82% identical to the 99 amino acid mouse Neublasmin and 81% identical to the 100 amino acid rat Neublasmin. Human and Chimpanzee Neublasmin differ by 1 amino acid residue in the mature part of the protein.

TABLE 1a
Percent sequence identity of full-length Neublasmin from human,
mouse and rat using Align0. The sequence identities are calculated
using scoring matrix: BLOSUM50, gap penalties: −12/−2

| | Human | Mouse | Rat |
|---|---|---|---|
| Human - 98 aa | 100 | | |
| Mouse - 99 aa | 81.8 | 100 | |
| Rat - 100 aa | 81.0 | 95.0 | 100 |

TABLE 1b
Percent sequence identity of mature Neublasmin from human, mouse
and rat using Align0. The sequence identities are calculated using
scoring matrix: BLOSUM50, gap penalties: −12/−2

| | Human | Mouse | Rat |
|---|---|---|---|
| Human - 68 aa | 100 | | |
| Mouse - 70 aa | 78.6 | 100 | |
| Rat - 71 aa | 78.9 | 94.4 | 100 |

Protein Processing:

Human Neublasmin contains an N-terminal signal peptide sequence of 30 amino acids which is cleaved at the sequence motif LRS-QP. This signal peptide cleavage site is clearly predicted by the SignalP method (“Identification of prokaryotic and eukaryotic signal peptides and prediction of their cleavage sites”. Nielsen H, Engelbrecht J, Brunak S, von Heijne G, Protein Engineering 10:1-6, 1997) and the output graph shown in the examples. Mouse and rat Neublasmin contain a predicted signal peptide of 28 amino acids, which is predicted to be cleaved at the sequence motif ALL-RP. As it is known in the art signal peptide processing is not always exactly as predicted and actual processing may vary from case to case. Actual cleavage site may be verified using state of the art N-terminal sequencing methods. A signal peptide cleavage site is found at a similar location in the mouse, rat, and Chimpanzee Neublasmin proteins.

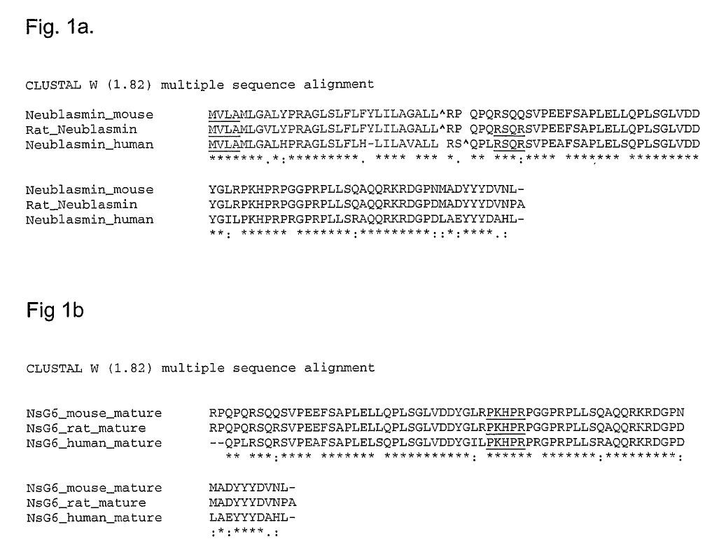

Furin cleavage is predicted to occur at the RSQR motif underlined in FIG. 1a. However, this motif is not conserved in mouse, and is therefore probably not physiologically relevant.

Furthermore, homologous recognition sites (underlined, FIG. 1b, ClustalW alignment) for the testicular germ-cell protease PC4 (proprotein convertase 4) are predicted in all Neublasmin proteins. PC4 has a reported sequence motif KXXR where X is any amino acid other than cysteine and it prefers proline at P3, P5 and/or P2′ positions (Basak et al., “In vitro elucidation of substrate specificity and bioassay of proprotein convertase 4 usign intramolecularly quenched fluorogenic peptides”, Biochem J. 380:505-514, 2004). PC4 expression is detectable after the 20th day of postnatal development in mice and mainly in the round spermatids (Nakayama et al. “Identification of the fourth member of the mammalian endoprotease family homologue to the yeast Kex2 protease”, J Biol Chem, 267:5897-5900, 1992). This corresponds precisely to the expression of Neublasmin in mouse testicles after birth (FIG. 8A). Similarly to PC4, Neublasmin expression is preserved into adulthood in mouse testicles. Thus two potentially bioactive Neublasmin peptides may be generated in each species.

Human Neublasmin N-peptide (38-40 residues, depending on exact signal peptide processing):

(SEQ ID NO. 26/27)

(RS)QPLRSQRSVPEAFSAPLELSQPLSGLVDDYGILPKHPR
Human Neublasmin C-peptide (30 residues):

(SEQ ID No. 28)

PRGPRPLLSRAQQRKRDGPDLAEYYYDAHL

(SEQ ID No. 29)

PRGPRPLLSRAHQRKRDGPDLAEYYYDAHL
(SNP variant)

Small bioactive peptides may be C-terminally amidated. Consensus sites are x-G-[RK]-[RK] [x is the amidation site]. No sites have been found in Neublasmin peptides.

The actual N-terminal of mature Neublasmin can be verified experimentally by C-terminal tagging using e.g. a C-terminal his tag, subsequent purification using a poly-his specific antibody or purification on a Ni column, and subsequent N-terminal sequencing of the purified protein. Purification of secreted peptides may also be done using antibodies directed against these peptides.

Another way of isolating Neublasmin polypeptides for N-terminal sequencing comprises size fractionation of proteins from conditioned medium from Neublasmin-expressing cells, or proteins from a biological sample such as sperm expected to contain one or more Neublasmin polypeptides. Proteins can be fractionated using e.g. gel-filtration and analysed using Neublasmin antibodies. Once the fractions containing Neublasmin polypeptide are identified, these can be used for N-terminal sequencing, as relatively pure fractions can be made.

Protein Function:

Neublasmin belongs to the category of proteins acting as hormones or growth factors. This notion is supported by predictions by the ProtFun protein function prediction server (Jensen, J L, Gupta R, Staerfeldt H H, Brunak S, 2003, “Prediction of human protein function according to Gene Ontology categories”, Bioinformatics 19(5):635-42), which provides very high odds scores (>30) for exactly this type of category. The odds scores for another known hormone and known Proprotein-convertase-4 substrate, propACAP (adenylate cyclase activating polypeptide precursor), and for other known secreted hormones are of the same magnitude or even lower.

| | Odds for Gene Ontology Category Hormone |
|---|---|
| Neublasmin | 31.0 |
| Insulin precursor | 10.0 |
| Neuropeptide Y precursor | 25.6 |
| Gastrin precursor | 38.3 |
| ProPACAP | 26.9 |

The ProtFun method predicts protein function based on sequence-derived features, but not the sequence itself. Features which are important for discriminating between the ‘hormone/growth factor’ classes versus all other classes are: protein sorting potential, protein targeting potential, signal peptide potential, low complexity regions, secondary protein structure, number of negative residues and number of atoms.

Gene Expression:

Development of testes in mice starts around day 10 post coitus when primordial germ cells migrate to the genital ridge and form a primitive gonad, tightly associated with the mesonephros (which later develops into kidney and epididymis). The primordial germ cells proliferate and Sertoli cells develop, embed, and nurse the primordial germ cells, which then differentiate into spermatogonia. After birth, spermatogonia start to proliferate and develop into spermacytes. Spermatocytes begin to undergo meiosis and differentiate into haploid, round spermatids around pn 22-24 (pn-days after birth, post natum). This timed and coordinated maturation of germ cells after birth implies that a given day after birth only a certain population of germ cell types will be present in the testis. The expected PCR product amplified from mNeublasmin cDNA was first detectable at pn22 and continued to be expressed throughout the remaining pn period analysed (FIG. 8A). This observation is consistent with the expression of Neublasmin being expressed stage-specifically in the germ cells and coexpressed with the PC4 endoprotease (Nakayama et al. “Identification of the fourth member of the mammalian endoprotease family homologue to the yeast Kex2 protease”, J Biol Chem, 267:5897-5900, 1992).

Testicular germ cell tumours (TGCT) are the most common malignancies among men aged 17-75 years. TGCT of young adults are classified into two main histological subtypes, classical seminoma and non-seminoma. These tumours arise from a common precursor, the carcinoma in situ (CIS). There is evidence that indicates that CIS is an inborn lesion, probably arising in early fetal life, which progresses to TGCT after puberty. However, the precise nature of the molecular mechanisms leading to CIS remains largely unknown. Morphologically, CIS cells are gonocyte-like intratubular germ cells that share several features with both seminoma and embryonal carcinoma. Expression of hNeublasmin has been detected in both normal testis and samples containing CIS by RT-PCR (as seen in FIG. 8B). In order to investigate which cell types that express Neublasmin, non-radioactive ISH on a testicular biopsy containing CIS was preformed. CIS cells contain large glycogen-rich vacuoles that disappear during fixation, thus the remnants of cytoplasm are usually visible as a thin ring attached to the cell membrane. As seen in FIG. 9, no signal or a very low signal was detected using the control probe (sense-probe). In contrast, expression in both CIS cells and in undifferentiated germinal cells was clearly detected with the anti-sense probe. The signal detected from the CIS cells was much stronger than from normal germ cells.

The expression data clearly demonstrate that expression of Neublasmin mRNA is up-regulated several folds in CIS compared to normal testes. As Neublasmin mRNA is also expressed in normal testes (albeit at a lower level), the difference in expression can be verified with quantitative PCR.

As a secreted protein Neublasmin may also be involved in the carcinogenic process either directly on the germ cells themselves (autocrine) or indirectly on other cell types (Sertoli cells) involved in regulating germ cell differentiation. Thus, it may be of therapeutic value to interfere with Neublasmin signalling in CIS for example by using a Neublasmin antagonist, such as an anti-Neublasmin antibody, a blocking peptide derived from Neublasmin, or a small molecule antagonist.

The gene expression patterns of human ES cell lines show many similarities with human germ cell tumours including human embryonal carcinomas and seminomas that are both derived from CIS cells (Sperger et al., 2003, PNAS, 100:13350-13355). Thus it is likely that Neublasmin may also have effects on human ES cells for example by inducing proliferation and/or antagonizing differentiation.

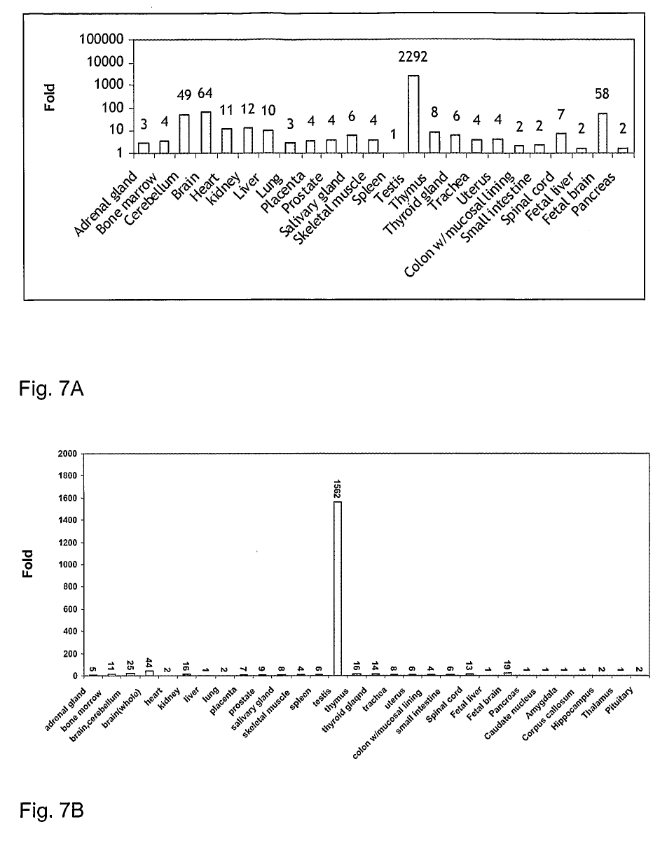

Human Neublasmin is also expressed in adult and foetal brain, more specifically in fetal mesencephalon (midbrain) (see the working Examples on Affymetrix Genechip studies) and in cerebellum (FIG. 7A). Neublasmin is also expressed at substantial levels in several sub-regions of the developing mouse CNS (FIG. 11). Based on the finding that Neublasmin is a secreted hormone/growth factor, which is expressed in the brain (cerebellum and Ventral and dorsal mesencephalon), the present inventors contemplate that Neublasmin has therapeutic potential in the treatment of disorders of the central nervous system, in particular in the treatment of neurodegenerative disorders. Similar expression patterns are found for a number of therapeutically very important secreted growth factors including GDNF, NGF, Neurturin, BDNF, NT4/5, NT3, Neublastin (Artemin). Verification of this neuroprotective function may be done using cellular assays such as the PC12 assay described in the appended examples, or in animal models of neurodegenerative disorders.

Consistent with the expression pattern of several important CNS related growth factors, Neublasmin expression is not only observed in the CNS but also in peripheral tissue, including skin, tumors, and in particular testis.

The therapeutic effect of Neublasmin may be mediated through an effect on growth, survival, and/or differentiation of targeted cells.

I Neublasmin Polypeptides

In a first aspect the invention relates to an isolated polypeptide for medical use, said polypeptide comprising an amino acid sequence selected from the group consisting of:

a) a mature form of the amino acid sequence selected from the group consisting of SEQ ID No. 9, 10, 14, 18, and 25;

b) a variant of a mature form of the amino acid sequence selected from the group consisting of SEQ ID No. 9, 10, 14, 18, and 25, wherein the variant has at least 50% sequence identity to said SEQ ID No.;

c) the amino acid sequence selected from the group consisting of SEQ ID No. 2, 4, 5, 7, 8, 12, 13, 16, 17, 23, and 24;

d) a variant of the amino acid sequence selected from the group consisting of SEQ ID No. 2, 4, 5, 7, 8, 12, 13, 16, 17, 23, and 24, wherein the variant has at least 50% sequence identity to said SEQ ID No.;

e) a mature Neublasmin polypeptide obtainable through expression of full length Neublasmin in a mammalian cell; and

f) a fragment of at least 20 contiguous amino acids of any of a) through d).

In addition to substantially full-length polypeptides, the present invention provides for biologically active variants of the polypeptides. A Neublasmin polypeptide or fragment is biologically active if it exhibits a biological activity of naturally occurring Neublasmin.

One particularly preferred biological activity is the ability to compete with wildtype Neublasmin in a receptor-binding assay. Preferably the wildtype Neublasmin is a Neublasmin C-terminal or N-terminal peptide resulting from the cleavage by proprotein-convertase-4.

Another particularly preferred biological activity is the ability to bind to an antibody, which is directed at an epitope, which is present on naturally occurring Neublasmin. Preferably the epitope is present on a Neublasmin C-terminal or N-terminal peptide.

A preferred biological activity is the ability to elicit substantially the same response as wildtype Neublasmin in the PC12 assay described in the Examples. By substantially the same response in the PC12 assay is intended that the number of neurite bearing cells is at least 50% of the number obtained with wildtype Neublasmin, more preferably at least 60%, more preferably at least 70%, more preferably at least 75%, more preferably at least 80%, more preferably at least 85%, more preferably at least 90%. The PC12 assay may also return the percentage improvement in survival over a control treatment. Substantially the same response in this context means an activity resulting in at least 50% of the improvement of wildtype Neublasmin, more preferably at least 60%, more preferably at least 70%, more preferably at least 75%, more preferably at least 80%, more preferably at least 90%. The biological activity of a variant Neublasmin may of course also be higher than that of the wildtype Neublasmin.

Variants can differ from naturally occurring Neublasmin in amino acid sequence or in ways that do not involve sequence, or both. Variants in amino acid sequence (“sequence variants”) are produced when one or more amino acids in naturally occurring Neublasmin is substituted with a different natural amino acid, an amino acid derivative or non-native amino acid. Particularly preferred variants include naturally occurring Neublasmin, or biologically active fragments of naturally occurring Neublasmin, whose sequences differ from the wild type sequence by one or more conservative amino acid substitutions, which typically have minimal influence on the secondary structure and hydrophobic nature of the protein or peptide. Variants may also have sequences, which differ by one or more non-conservative amino acid substitutions, deletions or insertions which do not abolish the Neublasmin biological activity.

Conservative substitutions typically include the substitution of one amino acid for another with similar characteristics such as substitutions within the following groups: valine, glycine; glycine, alanine; valine, isoleucine; aspartic acid, glutamic acid; asparagine, glutamine; serine, threonine; lysine, arginine; and phenylalanine, tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

Other conservative substitutions can be taken from Dayhoff: the Atlas of Protein Sequence and Structure (1988).

Other variants within the invention are those with modifications which increase peptide stability. Such variants may contain, for example, one or more nonpeptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D-instead of L-amino acids into the polypeptide may increase its resistance to proteases. See, e.g., U.S. Pat. No. 5,219,990. The peptides of this invention may also be modified by various changes such as insertions, deletions and substitutions, either conservative or nonconservative where such changes might provide for certain advantages in their use. Splice variants are specifically included in the invention.

In one embodiment, a variant Neublasmin comprises a naturally occurring allelic variant of the sequence selected from the group consisting of SEQ ID No 2, 4, 5, 7, 8, 9, 10, 12, 13, 14, 16, 17, 18, 22, 23, and 24. Said allelic variant sequence may be an amino acid sequence that is the translation of a nucleic acid sequence differing by a single nucleotide from a nucleic acid sequence selected from the group consisting of SEQ ID No 1, 3, 6, 11, and 15. SEQ ID No 6 is one human sequence that differs by a single nucleotide from the sequence of SEQ ID No 3 and results in a polypeptide (SEQ ID No. 7 or 8) differing by a single amino acid from SEQ ID No 4 or 5.

In other embodiments, variants with amino acid substitutions, which are less conservative can also result in desired derivatives, e.g., by causing changes in charge, conformation and other biological properties. Such substitutions would include for example, substitution of hydrophilic residue for a hydrophobic residue, substitution of a cysteine or proline for another residue, substitution of a residue having a small side chain for a residue having a bulky side chain or substitution of a residue having a net positive charge for a residue having a net negative charge.

The results of substitutions can partly be predicted using a Clustal W alignment as shown in FIG. 1. Normally it is safe to assume that a non-conserved, or semi-conserved residue can be changed to a residue found at the corresponding position in a Neublasmin polypeptide from another species without substantially altering the function of the polypeptide.

When the result of a given substitution cannot be predicted with certainty, the derivatives may be readily assayed according to the methods disclosed herein to determine the presence or absence of the desired characteristics.

Generally, substitutions that may be expected to induce changes in the functional properties of Neublasmin polypeptides are those in which: (i) a hydrophilic residue, e.g., serine or threonine, is substituted by a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative charge, e.g., glutamic acid or aspartic acid; or (iv) a residue having a bulky side chain, e.g., phenylalanine, is substituted for (or by) one not having such a side chain, e.g., glycine.

As mature Neublasmin polypeptide is predicted to be subject to proteolytic processing by proprotein-convertase-4, the bioactive polypeptide(s) may include the resulting C-terminal and/or N-terminal peptide. These peptides are described in more detail below.

In a preferred embodiment, the Neublasmin polypeptide is a Neublasmin peptide selected from the group consisting of

i) a peptide having an amino acid sequence selected from the group consisting of SEQ ID No 26, 27, 28, 29, 30, 31 32, 33, 34, and 35;

ii) a bioactive peptide having at least 60% sequence identity to a peptide selected from the group consisting of SEQ ID No 26, 27, 28, 29, 30, 31 32, 33, 34, and 35;

iii) a bioactive fragment of at least 15 contiguous amino acids of a peptide selected from the group consisting of SEQ ID No 26, 27, 28, 29, 30, 31 32, 33, 34, and 35;

iv) a peptide obtainable by pro-convertase-4 cleavage of a polypeptide having an amino acid sequence selected from the group consisting of SEQ ID No. 9, 10, 14, 18, and 25.

Preferably the variant bioactive peptide has at least 65% sequence identity, more preferably at least 70% sequence identity, more preferably at least 75% sequence identity, more preferably at least 80%, more preferably at least 85%, more preferably at least 90%, more preferably at least 95%, more preferably at least 98% sequence identity to a sequence selected from the group consisting of SEQ ID No 26, 27, 28, 29, 30, 31 32, 33, 34, and 35.

Preferably the bioactive peptide fragment contains at least 16, for example at least 17, such as at least 18, for example at least 19, such as at least 20, for example at least 21, such as at least 22, for example at least 23, such as at least 24, for example at least 25, such as at least 26, for example at least 27, such as at least 28, for example at least 29, such as at least 30 contiguous amino acids of a peptide selected from the group consisting of SEQ ID No 26, 27, 28, 29, 30, 31 32, 33, 34, and 35. In the case of fragments of N-terminal peptides these may constitute at least 31 contiguous amino acids of an N-terminal peptide, such as at least 32, for example at least 33, such as at least 34, for example at least 35, such as at least 36, for example at least 37, such as at least 38, for example at least 39 contiguous amino acids.

These peptides represent the C- and N-terminal peptides of Neublasmin obtained through proteolytic processing of mature Neublasmin by a the testes-specific proprotein-convertase-4. These are currently believed to be the bioactive Neublasmin peptides.

N-terminal Neublasmin peptides of the present invention include peptides selected from the group consisting of

i) a peptide having an amino acid sequence selected from the group consisting of SEQ ID No 26, 27, 30, 32, and 34;

ii) a bioactive peptide having at least 60% sequence identity to a peptide selected from the group consisting of SEQ ID No 26, 27, 30, 32, and 34; and

iii) a bioactive fragment of at least 15 contiguous amino acids from a peptide selected from the group consisting of SEQ ID No 26, 27, 30, 32, and 34.

The exact N-terminal of the bioactive N-terminal peptides depends on the exact cleavage of the signal peptide. As signal peptide cleavage is known to vary and as there is some uncertainty as to the exact position of the signal peptide cleavage site in particular in mouse and rat, the exact N-terminal may vary one or two amino acids from the predicted N-terminal of the predicted peptides.

Preferably the variant bioactive N-terminal peptide has at least 65% sequence identity, more preferably at least 70% sequence identity, more preferably at least 75% sequence identity, more preferably at least 80%, more preferably at least 85%, more preferably at least 90%, more preferably at least 95%, more preferably at least 98% sequence identity to a sequence selected from the group consisting of SEQ ID No 26, 27, 30, 32, and 34.

It is believed that both C-terminal and N-terminal peptides may be truncated in the N- and/or C-terminal to yield shorter, bioactive polypeptide fragments, which fragments retain the bioactivity of the full-length C-terminal or N-terminal peptides. Such truncated peptides may be used for raising antibodies against the full-length peptide or be used as Neublasmin analogues, which retain the signalling properties of full length C-terminal or N-terminal peptides.

Preferably the bioactive peptide fragment contains at least 16, for example at least 17, such as at least 18, for example at least 19, such as at least 20, for example at least 21, such as at least 22, for example at least 23, such as at least 24, for example at least 25, such as at least 26, for example at least 27, such as at least 28, for example at least 29, such as at least 30, for example at least 31, such as at least 32, for example at least 33, such as at least 34, for example at least 35, such as at least 36, for example at least 37, such as at least 38, for example at least 39 contiguous amino acids of a peptide having a sequence selected from the group consisting of SEQ ID No 26, 27, 30, 32, and 34.

C-terminal Neublasmin peptides of the present invention include peptides selected from the group consisting of

i) a peptide having an amino acid sequence selected from the group consisting of SEQ ID No 28, 29, 31, 33, and 35;

ii) a bioactive peptide having at least 60% sequence identity to a peptide selected from the group consisting of SEQ ID No 28, 29, 31, 33, and 35; and

iii) a bioactive fragment of at least 15 contiguous amino acids from a peptide selected from the group consisting of SEQ ID No 28, 29, 31, 33, and 35.

Preferably the variant bioactive C-terminal peptide has at least 65% sequence identity, more preferably at least 70% sequence identity, more preferably at least 75% sequence identity, more preferably at least 80%, more preferably at least 85%, more preferably at least 90%, more preferably at least 95%, more preferably at least 98% sequence identity to a sequence selected from the group consisting of SEQ ID No 28, 29, 31, 33, and 35.

Preferably the bioactive peptide fragment contains at least 16, for example at least 17, such as at least 18, for example at least 19, such as at least 20, for example at least 21, such as at least 22, for example at least 23, such as at least 24, for example at least 25, such as at least 26, for example at least 27, such as at least 28, for example at least 29 contiguous amino acids of a peptide having a sequence selected from the group consisting of SEQ ID No 28, 29, 31, 33, and 35.

Preferred Neublasmin peptides according to the invention are the human peptides, selected from the group consisting of Neublasmin peptides having an amino acid sequence selected from the group consisting of SEQ ID No. 26, 27, 28, and 29 and human peptides being the result of pro-convertase-4 cleavage of a mature human Neublasmin polypeptide, including SEQ ID No. 9 and 10.

Preferred C-terminal Neublasmin peptides include neublasmin peptides selected from the group consisting of Neublasmin peptides having an amino acid sequence selected from the group consisting of SEQ ID No. 28 and 29, which constitute human C-terminal peptides.

Preferred N-terminal Neublasmin peptides include neublasmin peptides selected from the group consisting of Neublasmin peptides having an amino acid sequence selected from the group consisting of SEQ ID No. 26 and 27, which constitute human N-terminal peptides.

Variants within the scope of the invention in one embodiment include proteins and peptides with amino acid sequences having at least fifty percent identity with human, chimpanzee, murine or rat Neublasmin (SEQ ID NO: 2, 4, 5 7, 8, 9, 10, 12, 13, 14, 16, 17, 18, 23, 24 and 25). More preferably the sequence identity is at least 60%, more preferably at least 65%, more preferably at least 70%, more preferably at least 75%, more preferably at least 80%, more preferably at least 85%, more preferably at least 90%, more preferably at least 95%, more preferably at least 98%.

In a preferred embodiment the sequence identity of the variant Neublasmin is determined with reference to a human Neublasmin polypeptide (SEQ ID No 2, 4, 7, 8, 9, and 10).

For the purposes of determining homology the minimum length of comparison sequences will generally be at least 8 amino acid residues, usually at least 12 amino acid residues. For the purposes of the present invention, the percent sequence identity is preferably calculated in a range of overlap of at least 25 amino acids, more preferably at least 30 amino acids, more preferably at least 35, more preferably at least 40, more preferably at least 45, more preferably at least 50, more preferably at least 55, more preferably at least 60, more preferably at least 68, the range being determined by BLASTP under default settings.

In one embodiment the percent sequence identity is calculated using global alignment, so that the variant and SEQ ID sequences are aligned, the total number of identical amino acid residues calculated and divided by the length of the SEQ ID NO.

In one embodiment, the variants include proteins comprising an amino acid sequence having at least 60% sequence identity to SEQ ID NO 2/4, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 5, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 7, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 8, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 9, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 10, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 12, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 13, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 14, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 16, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 17, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 18, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 23, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 24, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 25, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variant full length Neublasmin at corresponding positions comprises the residues marked in FIG. 1a as fully conserved (*), more preferably the variant full length Neublasmin also comprises at corresponding positions the residues marked in FIG. 1a as strongly conserved (: strongly conserved groups include: STA, NEQK, NHQK, NEDQ, QHRK, MILV, MILF, HY FYW), more preferably the variant full length Neublasmin also comprises at corresponding positions the residues marked in FIG. 1a as less conserved (. less conserved groups include: CSA, ATV, SAG, STNK, STPA, SGND, SNDEQK, NDEQHK, NEQHK, NEQHRK, VLIM, HFY).

In one embodiment, the variant mature Neublasmin at corresponding positions comprises the residues marked in FIG. 1b as fully conserved (*), more preferably the variant mature Neublasmin also comprises at corresponding positions the residues marked in FIG. 1b as strongly conserved (: strongly conserved groups include: STA, NEQK, NHQK, NEDQ, QHRK, MILV, MILF, HY FYW), more preferably the variant mature Neublasmin also comprises at corresponding positions the residues marked in FIG. 1b as less conserved (. less conserved groups include: CSA, ATV, SAG, STNK, STPA, SGND, SNDEQK, NDEQHK, NEQHK, NEQHRK, VLIM, HFY).

Just as it is possible to replace substituents of the protein, it is also possible to substitute functional groups, which are bound to the protein with groups characterized by similar features. Such modifications do not alter primary sequence.

These will initially be conservative, i.e., the replacement group will have approximately the same size, shape, hydrophobicity and charge as the original group.

Non-sequence modifications may include, for example, in vivo or in vitro chemical derivatization of portions of naturally occurring Neublasmin, as well as changes in acetylation, methylation, phosphorylation, carboxylation or glycosylation.

Also included within the invention are agents, which specifically bind to a protein of the invention, or a fragment of such a protein. These agents include Ig fusion proteins and antibodies (including single chain, double chain, Fab fragments, and others, whether native, humanized, primatized, or chimeric). Additional descriptions of these categories of agents are in PCT application WO 95/16709, the disclosure of which is herein incorporated by reference.

In addition, the protein may comprise a protein tag and optionally a protease cleavage site to allow subsequent purification and optionally removal of the tag. Non-limiting examples of affinity tags include a polyhis tag, a GST tag, a HA tag, a Flag tag, a C-myc tag, a HSV tag, a V5 tag, a maltose binding protein tag, a cellulose binding domain tag. Preferably for production and purification, the tag is a polyhistag. Preferably, the tag is in the C-terminal portion of the protein.

Specific antibodies to any Neublasmin are also useful in immunoassays to quantify the Neublasmin-substance for which a given antibody has specificity. Specific antibodies to a Neublasmin may also be bound to solid supports, such as beads or dishes, and used to remove the ligand from a solution, either for use in purifying the protein or in clearing it from the solution. Each of these techniques is routine to those of skill in the immunological arts.

A Neublasmin fusion protein can be used to allow imaging of tissues which express a receptor for Neublasmin, or in the immunohistological or preparative methods described above for antibodies to a Neublasmin.

Fusion proteins encompassing a Neublasmin polypeptide can be used to specifically target medical therapies against cells, which express a Neublasmin receptor.

The native signal sequence of Neublasmin may also be replaced in order to increase secretion of the protein in recombinant production in other mammalian cell types.

Neublasmin polypeptides may be produced through recombinant synthesis, in particular through recombinant expression in mammalian cells to ensure correct processing of the encoded polypeptide. To obtain C- and N-terminal peptides of Neublasmin, the mature protein may be exposed to proprotein-convertase-4 or the cells used for recombinant synthesis may comprise an additional expression construct coding for proprotein-convertase-4.

For recombinant expression of Neublasmin C- and N-terminal peptides the appropriate nucleic acid fragment corresponding to the desired peptide is cloned into an expression vector and inserted into a producer cell. For recombinant expression in a mammalian cell, a signal peptide coding sequence is fused to the sequence coding for the Neublasmin peptide.

Neublasmin C- and N-terminal peptides may also be synthesised using peptide synthesis, available from commercial producers of custom made peptides, including Biopeptide Co., LLC, 10457 ROSELLE ST STE I, SAN DIEGO, CA 92121-1510; AnaSpec, Inc., 2149 O'Toole Ave., San Jose, Calif. 95131, USA; and Genemed Synthesis Inc., 407 Cabot Road, South San Francisco, Calif. 94080, USA.

Synthetic methods to acquire peptides include: solid phase peptide synthesis and solution phase peptide synthesis.

Solid-phase peptide synthesis consists of three distinct sets of operations: 1) chain assembly on a resin; 2) simultaneous or sequential cleavage and deprotection of the resin-bound, fully protected chain; and 3) purification and characterisation of the target peptide. Various chemical strategies exist for the chain assembly and cleavage/deprotection operations, but purification and characterisation methods are more or less invariant to the methods used to generate the crude peptide product.

Two major chemistries for solid phase peptide synthesis are Fmoc (base labile protecting group) and t-Boc (acid labile a-amino protecting group). Each method involves fundamentally different amino acid side-chain protection and consequent cleavage/deprotection methods, and resins; t-Boc method requires use of stronger HF containing anisole alone or anisole plus other scavengers, where peptide-resins assembled by Fmoc chemistry usually cleaved by less harsh Reagents K or R. Fmoc chemistry is known for peptide synthesis of higher quality and in greater yield than t-Boc chemistry. Impurities in t-Boc-synthesized peptides mostly attributed to cleavage problems, dehydration and t-butylation. For peptide assembly HBTU/HOBt, carbodiimidemediated coupling and PyBOP/HOBt are the most popular routines. Peptides usually purified by reversed-phase HPLC (high performance liquid chromatography) using columns such as C-18, C-8, and C-4.

For large scale synthesis of well known peptides solution or liquid phase peptide synthesis can be applied. These “classical” methods for synthesis in solution are labour, time, and skill intensive largely due to the unpredictable solubility characteristics of intermediates

### II Neublasmin Nucleotide Sequences

The invention provides genomic and cDNA coding for Neublasmin, including for example the human genomic nucleotide sequence (SEQ ID No. 1) the nucleotide sequence of human, mouse and rat pre-Neublasmin cDNA (SEQ ID NO 3, 6, 11, and 15) and the sequences coding for mature Neublasmin of human, mouse, and rat origin (Nucleotides no 128-334 of SEQ ID NO 3 and 6, nucleotides no 119 to 331 of SEQ ID No. 11, and nucleotides 1 to 300 of SEQ ID No. 15). The genomic as well as the cDNA sequences may be used for recombinant expression of Neublasmin. For this purpose, however, the cDNA sequences are preferred as they are shorter. For the detection of Neublasmin expression, the genomic nucleotide sequence cannot be used.

Variants of these sequences are also included within the scope of the present invention.

The invention relates to an isolated nucleic acid molecule for medical use comprising a nucleic acid sequence encoding a polypeptide or its complementary sequence, said polypeptide comprising an amino acid sequence selected from the group consisting of:

a) a mature form of the amino acid sequence selected from the group consisting of SEQ ID No. 9, 10, 14, 18, and 25;

b) a variant of a mature form of the amino acid sequence selected from the group consisting of SEQ ID No. 9, 10, 14, 18, and 25, wherein the variant has at least 50% sequence identity to said SEQ ID No.;

c) the amino acid sequence selected from the group consisting of SEQ ID No. 2, 4, 5, 7, 8, 12, 13, 16, 17, 23, and 24;

d) a variant of the amino acid sequence selected from the group consisting of SEQ ID No. 2, 4, 5, 7, 8, 12, 13, 16, 17, 23, and 24, wherein the variant has at least 50% sequence identity to said SEQ ID No.; and

e) a fragment of at least 20 contiguous amino acids of any of a) through d).

The nucleic acid molecule may comprise the nucleotide sequence of a naturally occurring allelic nucleic acid variant.

The nucleic acid molecule of the invention may encode a variant polypeptide, wherein the variant polypeptide has the polypeptide sequence of a naturally occurring polypeptide variant.

In one embodiment the nucleic acid molecule differs by a single nucleotide from a nucleic acid sequence selected from the group consisting of SEQ ID No. 1, 3, 6, 11, 15, 19, 20, 21, and 22.

In a preferred embodiment, the nucleic acid encodes a Neublasmin peptide selected from the group consisting of

i) a peptide having an amino acid sequence selected from the group consisting of SEQ ID No 26, 27, 28, 29, 30, 31 32, 33, 34, and 35;

ii) a bioactive peptide having at least 60% sequence identity to a peptide selected from the group consisting of SEQ ID No 26, 27, 28, 29, 30, 31 32, 33, 34, and 35;

iii) a bioactive fragment of at least 15 contiguous amino acids of a peptide selected from the group consisting of SEQ ID No 26, 27, 28, 29, 30, 31 32, 33, 34, and 35;

iv) a peptide obtainable by pro-convertase-4 cleavage of a polypeptide having an amino acid sequence selected from the group consisting of SEQ ID No. 9, 10, 14, 18, and 25.

Preferably the nucleic acid encoding a variant bioactive peptide encodes a peptide having at least 65% sequence identity, more preferably at least 70% sequence identity, more preferably at least 75% sequence identity, more preferably at least 80%, more preferably at least 85%, more preferably at least 90%, more preferably at least 95%, more preferably at least 98% sequence identity to a sequence selected from the group consisting of SEQ ID No 26, 27, 28, 29, 30, 31 32, 33, 34, and 35.

Preferably the nucleic acid encoding a bioactive peptide fragment codes for a peptide containing at least 16, for example at least 17, such as at least 18, for example at least 19, such as at least 20, for example at least 21, such as at least 22, for example at least 23, such as at least 24, for example at least 25, such as at least 26, for example at least 27, such as at least 28, for example at least 29, such as at least 30 contiguous amino acids of a peptide selected from the group consisting of SEQ ID No 26, 27, 28, 29, 30, 31 32, 33, 34, and 35. In the case of fragments of N-terminal peptides these may constitute at least 31 contiguous amino acids of an N-terminal peptide, such as at least 32, for example at least 33, such as at least 34, for example at least 35, such as at least 36, for example at least 37, such as at least 38, for example at least 39 contiguous amino acids.

In one embodiment the nucleic acid encodes N-terminal Neublasmin peptides selected from the group consisting of

i) a peptide having an amino acid sequence selected from the group consisting of SEQ ID No 26, 27, 30, 32, and 34;

ii) a bioactive peptide having at least 60% sequence identity to a peptide selected from the group consisting of SEQ ID No 26, 27, 30, 32, and 34; and

iii) a bioactive fragment of at least 15 contiguous amino acids from a peptide selected from the group consisting of SEQ ID No 26, 27, 30, 32, and 34.

The exact N-terminal of the bioactive N-terminal peptides depends on the exact cleavage of the signal peptide. As signal peptide cleavage is known to vary and as there is some uncertainty as to the exact position of the signal peptide cleavage site in particular in mouse and rat, the exact N-terminal may vary one or two amino acids from the predicted N-terminal of the predicted peptides.

Preferably the nucleic acid encoding a variant bioactive N-terminal peptide encodes a peptide having at least 65% sequence identity, more preferably at least 70% sequence identity, more preferably at least 75% sequence identity, more preferably at least 80%, more preferably at least 85%, more preferably at least 90%, more preferably at least 95%, more preferably at least 98% sequence identity to a sequence selected from the group consisting of SEQ ID No 26, 27, 30, 32, and 34.

Preferably the nucleic acid encodes a bioactive peptide fragment containing at least 16, for example at least 17, such as at least 18, for example at least 19, such as at least 20, for example at least 21, such as at least 22, for example at least 23, such as at least 24, for example at least 25, such as at least 26, for example at least 27, such as at least 28, for example at least 29, such as at least 30, for example at least 31, such as at least 32, for example at least 33, such as at least 34, for example at least 35, such as at least 36, for example at least 37, such as at least 38, for example at least 39 contiguous amino acids of a peptide having a sequence selected from the group consisting of SEQ ID No 26, 27, 30, 32, and 34.

In one embodiment, nucleic acid encodes a C-terminal Neublasmin peptide selected from the group consisting of

i) a peptide having an amino acid sequence selected from the group consisting of SEQ ID No 28, 29, 31, 33, and 35;

ii) a bioactive peptide having at least 60% sequence identity to a peptide selected from the group consisting of SEQ ID No 28, 29, 31, 33, and 35; and

iii) a bioactive fragment of at least 15 contiguous amino acids from a peptide selected from the group consisting of SEQ ID No 28, 29, 31, 33, and 35.

Preferably the nucleic acid encoding a variant bioactive C-terminal peptide encodes a peptide having at least 65% sequence identity, more preferably at least 70% sequence identity, more preferably at least 75% sequence identity, more preferably at least 80%, more preferably at least 85%, more preferably at least 90%, more preferably at least 95%, more preferably at least 98% sequence identity to a sequence selected from the group consisting of SEQ ID No 28, 29, 31, 33, and 35.

Preferably the nucleic encoding a bioactive peptide fragment encodes a peptide containing at least 16, for example at least 17, such as at least 18, for example at least 19, such as at least 20, for example at least 21, such as at least 22, for example at least 23, such as at least 24, for example at least 25, such as at least 26, for example at least 27, such as at least 28, for example at least 29 contiguous amino acids of a peptide having a sequence selected from the group consisting of SEQ ID No 28, 29, 31, 33, and 35.

Preferred Neublasmin polynucleotides encode human peptides, selected from the group consisting of Neublasmin peptides having an amino acid sequence selected from the group consisting of SEQ ID No. 26, 27, 28, and 29 and human peptides being the result of pro-convertase-4 cleavage of a mature human Neublasmin polypeptide, including SEQ ID No. 9 and 10.

Preferred Neublasmin polynucleotides encoding C-terminal Neublasmin peptides encode peptides peptides selected from the group consisting of Neublasmin peptides having an amino acid sequence selected from the group consisting of SEQ ID No. 28 and 29, which constitute human C-terminal peptides.

Preferred Neublasmin polynucleotides encoding N-terminal Neublasmin peptides encode neublasmin peptides selected from the group consisting of Neublasmin peptides having an amino acid sequence selected from the group consisting of SEQ ID No. 26 and 27, which constitute human N-terminal peptides.

Preferably the encoded polypeptide has at least 60% sequence identity to a sequence selected from the group consisting of SEQ ID No. 5, 8, 13, 17, and 24, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID Nos. Said sequences include the N-4 polypeptides.

In a preferred embodiment the encoded polypeptide has at least 60% sequence identity to a sequence selected from the group consisting of SEQ ID No. 2, 4, 5, 7, 8, 9, and 10, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID Nos. Said sequences constitute human Neublasmin polypeptides.

In a preferred embodiment the encoded polypeptide has at least 60% sequence identity to a sequence selected from the group consisting of SEQ ID No. 9 and 10, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID Nos. Said sequences constitute human mature Neublasmin polypeptides.

In one aspect the nucleic acid molecule comprises a nucleotide sequence selected from the group consisting of

a) the nucleotide sequence selected from the group consisting of SEQ ID No. 1, 3, 6, 11, 15, 19, 20, 21, and 22;

b) a nucleotide sequence having at least 50% sequence identity to a nucleotide sequence selected from the group consisting of SEQ ID No. 1, 3, 6, 11, 15, 19, 20, 21, and 22;

c) a nucleic acid sequence of at least 60 contiguous nucleotides of a sequence selected from the group consisting of SEQ ID No. 1, 3, 6, 11, 15, 19, 20, 21, and 22;

c) the complement of a nucleic acid capable of hybridising with nucleic acid having the sequence selected from the group consisting of SEQ ID No.: 1, 3, 6, 11, 15, 19, 20, 21, and 22 under conditions of high stringency; and

d) the nucleic acid sequence of the complement of any of the above.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, most preferred at least 95% identity to a polynucleotide sequence presented as SEQ ID NO: 1, 3, 6, 11 or 15, when determined over the length of the coding sequence of the SEQ ID NO:.

In one embodiment, the isolated polynucleotide of the invention has at least 60, more preferably at least 65%, more preferably at least 70%, more preferably at least 75%, more preferably at least 80%, preferably at least 85%, more preferred at least 90%, most preferred at least 95% identity to the polynucleotide sequence presented as SEQ ID NO: 1, when determined over the entire length of the SEQ ID NO:, more preferably when determined over the length of the coding sequence.

In one embodiment, the isolated polynucleotide of the invention has at least 60, more preferably at least 65%, more preferably at least 70%, more preferably at least 75%, more preferably at least 80%, preferably at least 85%, more preferred at least 90%, most preferred at least 95% identity to the polynucleotide sequence presented as SEQ ID NO: 3, when determined over the entire length of the SEQ ID NO:, more preferably when determined over the length of the coding sequence.

In one embodiment, the isolated polynucleotide of the invention has at least 60, more preferably at least 65%, more preferably at least 70%, more preferably at least 75%, more preferably at least 80%, preferably at least 85%, more preferred at least 90%, most preferred at least 95% identity to the polynucleotide sequence presented as SEQ ID NO: 6, when determined over the entire length of the SEQ ID NO., more preferably when determined over the length of the coding sequence.

In one embodiment, the isolated polynucleotide of the invention has at least 60, more preferably at least 65%, more preferably at least 70%, more preferably at least 75%, more preferably at least 80%, preferably at least 85%, more preferred at least 90%, most preferred at least 95% identity to the polynucleotide sequence presented as SEQ ID NO: 11, when determined over the entire length of the SEQ ID NO., more preferably when determined over the length of the coding sequence.

In one embodiment, the isolated polynucleotide of the invention has at least 60, more preferably at least 65%, more preferably at least 70%, more preferably at least 75%, more preferably at least 80%, preferably at least 85%, more preferred at least 90%, most preferred at least 95% identity to the polynucleotide sequence presented as SEQ ID NO: 15, when determined over the entire length of the SEQ ID NO., more preferably when determined over the length of the coding sequence.

In addition, the compounds of the invention include sequences, which are derivatives of these sequences. The invention also includes vectors, liposomes and other carrier vehicles, which encompass one of these sequences or a derivative of one of these sequences. The invention also includes proteins transcribed and translated from Neublasmin cDNA, preferably human Neublasmin cDNA, including but not limited to human Neublasmin and derivatives and variants.

In another embodiment, the invention relates to the use of the nucleic acids and proteins of the present invention to design probes to isolate other genes, which encode proteins with structural or functional properties of the Neublasmin proteins of the invention. The probes can be a variety of base pairs in length. For example, a nucleic acid probe can be between about 10 base pairs in length to about 150 base pairs in length.

Alternatively, the nucleic acid probe can be greater than about 150 base pairs in length. Exceptional methods are provided in Ausubel et al., “Current Protocols in Molecular Biology”, J. Wiley (ed.) (1999), the entire teachings of which are herein incorporated by reference in their entirety.

The design of the oligonucleotide (also referred to herein as nucleic acid) probe should preferably follow these parameters:
i) it should be designed to an area of the sequence which has the fewest ambiguous bases, if any and
ii) it should be designed to have a calculated Tₘ of about 80° C. (assuming 2° C. for each A or T and 4° C. for each G or C).

The oligonucleotide should preferably be labeled to facilitate detection of hybridisation. Labelling may be with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4×10⁶ dpm/pmole. The bacterial culture containing the pool of full-length clones should preferably be thawed and 100 μL of the stock used to inoculate a sterile culture flask containing 25 ml of sterile L-broth containing ampicillin at 100 pg/ml, preferably at 100 μg/mL.

The culture should preferably be grown to saturation at about 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 pg/ml, preferably at 100 μg/mL, and agar at 1.5% in a 150 mm petri dish when grown overnight at about 37° C. Other known methods of obtaining distinct, well-separated colonies can also be employed.

Standard colony hybridization procedures should then be used to transfer the colonies to nitrocellulose filters and lyse, denature and bake them. Highly stringent (also referred to herein as “high stringency”) conditions are those that are at least as stringent as, for example, 1×SSC at about 65° C., or 1×SSC and 50% formamide at about 42° C. “Moderate stringency” conditions are those that are at least as stringent as 4×SSC at about 65° C., or 4×SSC and 50% formamide at about 42° C. “Reduced stringency” conditions are those that are at least as stringent as 4×SSC at about 50° C., or 6×SSC and 50% formamide at 40° C.

The filter is then preferably incubated at about 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 g/ml of yeast RNA, and 10 mM EDTA (approximately 10 mL per 150 mm filter). Preferably, the probe is then added to the hybridization mix at a concentration greater than or equal to 1×10⁶ dpm/mL. The filter is then preferably incubated at about 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at about 65° C. for 30 minutes to 1 hour is optional. The filter is then preferably dried and subjected to autoradiography for sufficient time to visualize the positives on the X-ray film. Other known hybridization methods can also be employed. The positive colonies are then picked, grown in culture, and plasmid DNA isolated using standard procedures. The clones can then be verified by restriction analysis, hybridization analysis, or DNA sequencing.

Suitable experimental conditions for determining hybridization between a nucleotide probe and a homologous DNA or RNA sequence, involves pre-soaking of the filter containing the DNA fragments or RNA to hybridize in 5×SSC [Sodium chloride/Sodium citrate; cf. Sambrook et al.; Molecular Cloning: A Laboratory Manual, Cold Spring Harbor Lab., Cold Spring Harbor, N.Y. 1989] for 10 minutes, and pre-hybridization of the filter in a solution of 5×SSC, 5×Denhardt's solution [cf. Sambrook et al.; Op cit.], 0.5% SDS and 100 μg/ml of denatured sonicated salmon sperm DNA [cf. Sambrook et al.; Op cit.], followed by hybridization in the same solution containing a concentration of 10 ng/ml of a random-primed [Feinberg A P & Vogelstein B; Anal. Biochem. 1983 132 6-13], ³²P-dCTP-labeled (specific activity>1×10⁹ cpm/μg) probe for 12 hours at approximately 45° C. The filter is then washed twice for 30 minutes in 0.1×SSC, 0.5% SDS at a temperature of at least at least 60° C. (medium stringency conditions), preferably of at least 65° C. (medium/high stringency conditions), more preferred of at least 70° C. (high stringency conditions), and even more preferred of at least 75° C. (very high stringency conditions). Molecules to which the oligonucleotide probe hybridizes under these conditions may be detected using a x-ray film.

In yet another embodiment, the invention relates to nucleic acid sequences (e.g., DNA, RNA) that hybridise to nucleic acids of Neublasmin. In particular, nucleic acids which hybridise to SEQ ID NO: 1, SEQ ID NO: 3, SEQ ID NO:6, SEQ ID NO:11, or SEQ ID No. 15 under high, moderate or reduced stringency conditions as described above.

In still another embodiment, the invention relates to a complement of nucleic acid of Neublasmin. In particular, it relates to complements of SEQ ID NO: 1, SEQ ID NO: 3, SEQ ID NO: 6, SEQ ID No. 11, and SEQ ID NO 15.

In another embodiment, the invention relates to an RNA counterpart of the DNA nucleic acid of Neublasmin. In particular, it relates to RNA counterparts of SEQ ID NO: 1, SEQ ID NO: 3, SEQ ID NO 6, SEQ ID NO 11 or SEQ ID NO: 15.

Codon optimised nucleic acid molecules for enhanced expression in selected host cells, including but not limited to E. coli, yeast species, Chinese Hamster, Baby Hamster, insect, and fungus are also contemplated.

Variant nucleic acids can be made by state of the art mutagenesis methods. Methods for shuffling coding sequences from human with those of mouse, rat or chimpanzee are also contemplated. Specifically a shuffled variant may be between SEQ ID No 19 or 20 on one hand and 21 and/or 22 on the other hand. Also included are shuffled variants between SEQ ID No 3 or 6 and 11 and/or 15.

III Therapeutic Uses of the Compounds of the Invention

In one aspect the invention relates to a pharmaceutical composition comprising

i) the polypeptide of the invention; or

ii) the isolated nucleic acid sequence of the invention; or

iii) the expression vector of the invention; or

iv) a composition of host cells according to the invention; or

v) a packaging cell line according to the invention; or

vi) an implantable cell culture device according to the invention; or

vii) a biocompatible capsule according to the invention; and

viii) a pharmaceutically acceptable carrier.

In a further aspect the invention relates to the use of

i) the polypeptide according to the invention; or

ii) the isolated nucleic acid sequence according to the invention; or

iii) the expression vector according to the invention; or

iv) a composition of host cells according to the invention;

v) an implantable cell culture device according to the invention; or

vi) a biocompatible capsule according to the invention; or

vii) a packaging cell line according to the invention;

for the manufacture of a medicament.

In a further aspect the invention relates to a method of treatment of a pathological condition in a subject, comprising administering to a subject in need thereof a therapeutically effective amount of:

i) the polypeptide according to the invention; or

ii) the isolated nucleic acid sequence according to the invention; or

iii) the expression vector according to the invention; or

iv) a composition of host cells according to the invention;

v) an implantable cell culture device according to the invention; or

vi) a biocompatible capsule according to the invention; or

vii) a packaging cell line according to the invention.

By treatment is intended not only preventive or curative treatment but also ameliorative, symptomatic and/or prophylactic treatment.

In one preferred embodiment the invention relates to a pathological condition related to testis. This embodiment is based on the present inventor's finding of Neublasmin expression in testis, and in particular based on the observed temporal regulation of Neublasmin expression in the developing and adult testes. The expression in testis has been confirmed by the immunohistochemistry data shown in FIG. 15, showing that Neublasmin protein is specifically present in high quantities in spermatoids and mature spermatocytes.

This embodiment is further based on the present inventors' finding of up-regulated Neublasmin expression in carcinoma in situ and in further stages of germ cell tumours. Examples of diseases included within the scope of this embodiment include male sterility, impotence, erectile dysfunction, cancer, and germ cell tumours. In a preferred embodiment the testes reates disorder is male sterility or infertility.

Neublasmin may in particular be administered to an individual with reduced Neublasmin expression as is e.g. the case for insulin administration to diabetics and EPO administration to subjects with anemia, and human growth hormone to subjects suffering from dwarfism. Neublasmin may also be provided as a therapeuticum to individuals with normal neublasmin expression. There are also examples of hormones/growth factors having a therapeutic effect, wherein the patients do not have a lowered level of the hormone/growth factor. Examples include GDNF or Neurturin for Parkinson's Disease and NGF for Alzheimer's Disease.

Native and variant Neublasmin, anti-Neublasmin antibodies, and fusion proteins comprising Neublasmin may be used to stimulate testicle cell growth, differentiation, and/or survival in disease situations where these cells are lost or damaged. Examples of target cells for neublasmin therapy include sperm cells, spermagononia, spermatogonial stem cells, and carcinoma in situ, testicular tumour cells, including seminoma, teratoma, and embryonal carcinoma.

In a preferred embodiment, the invention relates to a pathological condition associated with the nervous system including the CNS. This is based on the finding of expression of Neublasmin in the human foetal and adult brain, in cerebellum and the developing human central midbrain. This expression is also found in the developing mouse CNS, in which expression was hightes in cerebellum and the central midbrain (Dorsal mesencephalon). Similar expression levels were found in spinal cord and the striatum. Expression in the developing mouse CNS indicates that expression increases in the sub-regions at around the time of terminal differentiation and remains relatively high into adulthood. In one embodiment, the invention relates to a neurodegenerative disease.

In one embodiment, said neurodegenerative disease is a disease involving lesioned and/or injured neurons, such as traumatic lesions of peripheral nerves, the medulla, the spinal cord, cerebral ischaemic neuronal damage.

In another embodiment the neurodegenerative disease is a disease involving peripheral neuropathy and associated neuropathic pain.

In another embodiment the neurodegenerative disease is Alzheimer's disease. In another embodiment the neurodegenerative disease is Huntington's disease. In another embodiment the neurodegenerative disease is Parkinson's disease. In another embodiment the neurodegenerative disease is amyotrophic lateral sclerosis. In another embodiment the neurodegenerative disease is memory impairment connected to dementia. In another embodiment the neurodegenerative disease is an excitotoxic disease selected from the group consisting of ischaemia, epilepsy, and trauma due to injury, cardiac arrest or stroke.

In one embodiment, the polypeptides, nucleic acids, expression vectors, capsules, and compositions of the invention are used in the treatment of disorders, diseases, or damages associated with the Cerebellum, including but not limited to sensory ataxia, multiple sclerosis, neurodegenerative spinocerebellar disorders, hereditary ataxia, cerebellar atrophies (such as Olivopontocerebellar Atrophy (OPCA), Shy-Drager Syndrome (multiple systems atrophy)), and alcoholism. This function is supported by expression in the cerebellum combined with the bioinformatics analyses. Verification of this function may be done with assays testing Protection of cerebellar granule cells from glutamate toxicity and potassium deprivation.

Other CNS related growth factors, which are secreted, which are predicted to act as growth factors/hormones by function prediction programs such as ProtFun, have important therapeutic uses in various eye indications associated with loss of cells in retina and/or cornea. E.g NGF, a candidate for Alzheimer's disease, has been used for the treatment of corneal ulcer (U.S. Pat. No. 6,063,757 and EP 0 973 872). Neublastin (Artemin), a candidate for peripheral neuropathy, and GDNF, a candidate for Parkinson's Disease, have been used for wound healing, in particular in cornea (EP 1 223 966). The present inventors therefore contemplate the use of Neublasmin, and variants thereof for the treatment of eye disorders.

Confirmation of such use can be obtained partly by expression analysis in eye tissues (cornea, retina) and more specifically by using various state of the art in vitro assays (retinal explant assays, corneal cultures). Verification of function may also be performed in state of the art animal models for corneal wounds (corneal lesion in rabbits) and retina (retinitis pigmentosa mutant models available for mouse and rat).

Therefore, in one aspect of the invention the polypeptides, nucleic acids, expression vectors, capsules, and compositions of the present invention can also be used for the treatment of eye diseases, such as retinitis pigmentosa, macular degeneration, glaucoma, and diabetic retinopathy.

Native and variant Neublasmin, anti-Neublasmin antibodies, and fusion proteins comprising Neublasmin may have therapeutic utility. Neublasmin may be used to stimulate neuronal cell growth, differentiation, and/or survival in disease situations where these cells are lost or damaged.

In one embodiment, the genes and proteins of the invention may be used to treat conditions where neural growth and regeneration is desirable. This would include any conditions involving disorders of neural degeneration, such as Alzheimer's disease, Parkinson's, Huntington's, Tourette's, amyotrophic lateral sclerosis, as well as motor neuron disease, demyelinating diseases such as multiple sclerosis. Also included are disorders of damage to neural tissue, whether caused by neoplastic impingement, trauma, or cerebrovascular events such as hemorrhage or emboli. Diseases of the cranial nerves and of the spinal cord, including disorders involving traumatic, inflammatory, congenital or vascular etiologies, are specifically included, as are disorders affecting the autonomic nervous system. Also included are developmental neural disorders such as mental retardation, autism, fetal alcohol syndrome, Down's syndrome, and cerebral palsy. The compounds of the invention may also be used to treat syndromes involving the peripheral nervous system, particularly peripheral neuropathies. These disorders include those caused by any of the factors previously listed, and specifically include Lyme disease, HIV-associated neuropathies, polymyositis, muscular dystrophy, and myasthenia gravis.

The compounds of the invention are administered in therapeutically-effective amounts, which means an amount of a compound which produces a medically desirable result or exerts an influence on the particular condition being treated.

The term “subject” used herein is taken to mean any mammal to which Neublasmin protein or gene may be administered. Subjects specifically intended for treatment with the method of the invention include humans, as well as nonhuman primates, sheep, horses, cattle, goats, pigs, dogs, cats, rabbits, guinea pigs, hamsters, gerbils, rats and mice, as well as the organs, tumors, and cells derived or originating from these hosts.

Other therapeutic aspects of the invention include ways of inhibiting the activity of Neublasmin polypeptides in germ cell tumours, including carcinoma in situ, seminoma and non-seminoma, including embryonal carcinoma, teratoma and teratocarcinoma.

The reduction in neublasmin activity may be achieved in several ways, in particular through the administration of a Neublasmin antagonist. Neublasmin antagonists include therapeutic antibodies capable of binding a Neublasmin polypeptide and/or a Neublasmin C- or N-terminal peptide. Further antagonists include modified peptides capable of inhibiting signalling through a Neublasmin receptor. Interfering RNA is also an example of Neublasmin antagonists. Further examples include inhibitors of proprotein-convertase-4. By inhibiting proprotein-convertase-4, the processing of Neublasmin is inhibited and the bioactive peptides are not produced. An inhibitor of proprotein-convertase-4 can be a small peptide designed to match a proprotein-convertase-4 site. A particularly preferred proprotein-convertase-4 inhibitor includes the pentapeptide PKHPR, based on the proprotein-convertase-4 site in Neublasmin. Other proconveratase-4 inhibitors can be based on other known substrates of the proprotein-convertase, disclosed in Basak et al 2004 (Biochem J, 380:505-514), e.g. PAKSAR, PAKSER, RVKNKGRR, TIAERGRLMR, EPGRQSEMRR, FMSKERMMR, ERKPAR, and EPKPAR. Other peptides include peptides with the motif: KX₁KX₂X₃R or KX₂X₃R, where X₁, X₂, and X₃ individually is any amino acid other than cysteine and X₁ and X₂ preferably are proline. Other small molecule proprotein-convertase-4 inhibitors may also be used for reducing the activity of Neublasmin polypeptides.

In another embodiment of the invention, a Neublasmin antagonist as described above is used as a male contraceptive as the present inventors believe that Neublasmin peptides play a role in spermatogenesis, and that impaired Neublasmin processing may be responsible for the aberrant fertility in PC-4 null mice. A possible male contraceptive includes antibodies against Neublasmin polypeptides or C- and/or N-terminal peptides. It may also be possible to obtain male sterility by immunising male subjects with antigenic Neublasmin.

In a further aspect, the invention relates to a method of treatment of germ cell cancer, including carcinoma in situ, seminoma and non-seminoma including embryonal carcinoma, teratoma, and teratocarcinoma. The increased level of expression of Neublasmin in carcinoma in situ may be a mere symptom of the cancerous condition of the cells, by it is also likely that the increased expression in itself may contribute to the progress of the cancer. Therefore, a reduction of the level of expression of Neublasmin or an inactivation of bioactive Neublasmin peptides may ameliorate the cancer.

IIIB. In Vitro Uses of the Compounds of the Invention

As Neublasmin is a secreted growth factor expressed in the testes and various stages of germ cell tumours and is believed to be involved in spermatogenesis, one aspect of the invention relates to use of a Neublasmin polypeptide, in particular a C- or N-terminal Neublasmin peptide as a growth factor in mammalian cell cultures in vitro. The effect of a Neublasmin polypeptide may be on survival, division, or differentiation of the cells.

Neublasmin polypeptides may be used as a growth factor for cultures comprising mammalian stem cells, including multipotent and pluripotent stem cells, in particular spermatogonial stem cells.

PC-4 knock-out mice have deficient fertility. In PC-4 knock out mice, Neublasmin processing is blocked and bioactive peptides are not produced. The present inventors believe that the absence of Neublasmin peptides may be part of the reason for the reduced fertility in PC-4 deficient mice. Therefore, Neublasmin polypeptides may be used for stimulating sperm cells (and consequently stimulating fertility) prior to or during in vitro fertilisation or insemination. In one embodiment, a Neublasmin polypeptide is administered to a subject prior to obtaining a sperm sample from said individual. In another embodiment, a Neublasmin polypeptide is administered in vitro to a sperm/semen sample after it has been obtained from a subject. In embodiments of the invention, neublasmin peptides are used for stimulating sperm cells from animals, including horse, cattle, pig, dog, and cat.

IV. Therapeutic Formulations and Administration

The compounds of the invention may be administered in any manner, which is medically acceptable. This may include injections, by parenteral routes such as intravenous, intravascular, intraarterial, subcutaneous, intramuscular, intratumor, intraperitoneal, intraventricular, intraepidural, intertracheal, or others as well as nasal, ophthalmic, rectal, or topical. Sustained release administration is also specifically included in the invention, by such means as depot injections or erodible implants. Peroral administration is also conceivable provided the protein is protected against degradation in the stomach.

For the treatment of testicular disorders, localised is particularly preferred, because of the testis-blood barrier. Localised delivery to the testes can be used both for proteins, for antibodies, and for in vivo gene therapy. Encapsulated cells secreting Neublasmin can also be implanted into the testes for prolonged localised delivery.

Localized delivery is particularly contemplated, by such means as delivery via a catheter to one or more arteries, such as the ophthalmic artery, or via catheter for localised delivery to locations in the CNS, preferably the ventral and/or dorsal mesencephalon, to the cerebellum or to striatum, or to testes. Methods for local delivery of protein formulations to the CNS are described in U.S. Pat. No. 6,042,579 (Medtronic). Another type of localised delivery comprises delivery using encapsulated cells (see section below).

The term “pharmaceutically acceptable carrier” means one or more organic or inorganic ingredients, natural or synthetic, with which the Neublasmin is combined to facilitate its application. A suitable carrier includes sterile saline although other aqueous and non-aqueous isotonic sterile solutions and sterile suspensions known to be pharmaceutically acceptable are known to those of ordinary skill in the art. In this regard, the term “carrier” encompasses liposomes and the HIV-1 tat protein (See Chen et al., Anal. Biochem. 227: 168-175, 1995) as well as any plasmid and viral expression vectors. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

The liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154. In addition, it may be desirable to express the novel polypeptides of this invention, as well as other selected polypeptides, as lipoproteins, in order to enhance their binding to liposomes. The recombinant Neublasmin protein is purified, for example, from CHO cells by immunoaffinity chromatography or any other convenient method, then mixed with liposomes and incorporated into them at high efficiency. The liposome-encapsulated protein may be tested in vitro for any effect on stimulating cell growth.

Any of the Neublasmin polypeptides of this invention may be used in the form of a pharmaceutically acceptable salt. Suitable acids and bases which are capable of forming salts with the polypeptides of the present invention are well known to those of skill in the art, and include inorganic and organic acids and bases.

In one embodiment the invention relates to a pharmaceutical composition comprising a Neublasmin protein according to the invention, a polynucleotide coding for a Neublasmin, a vector comprising said polynucleotide sequence, or a packaging cell line capable of producing a viral vector comprising said polynucleotide sequence.

In addition to the active ingredients, the pharmaceutical compositions may comprise suitable ingredients. Further details on techniques for formulation and administration may be found in the latest edition of Remington's Pharmaceutical Sciences (Maack Publishing Co., Easton, Pa.).

When in vivo administration of a Neublasmin polypeptide is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a Neublasmin polypeptide is desired in a formulation with release characteristics suitable for the treatment of any disease or disorder requiring administration of the Neublasmin polypeptide, microencapsulation of the Neublasmin polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

The dose administered must of course be carefully adjusted to the age, weight and condition of the individual being treated, as well as the route of administration, dosage form and regimen, and the result desired, and the exact dosage should of course be determined by the practitioner.

V. Pharmaceutical Preparations for Gene Therapy

To form a Neublasmin composition for gene therapy use in the invention, Neublasmin encoding expression viral vectors may be placed into a pharmaceutically acceptable suspension, solution or emulsion. Suitable mediums include saline and liposomal preparations.

More specifically, pharmaceutically acceptable carriers may include sterile aqueous of non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils.

Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like. Further, a composition of Neublasmin transgenes may be lyophilized using means well known in the art, for subsequent reconstitution and use according to the invention.

A colloidal dispersion system may also be used for targeted gene delivery.

Colloidal dispersion systems include macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley, et al., Trends Biochem. Sci., 6: 77, 1981). In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the Neublasmin at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6: 682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further example of a delivery system includes transplantation into the therapeutic area of a composition of packaging cells capable of producing vector particles as described in the present invention. Methods for encapsulation and transplantation of such cells are known in the art, in particular from WO 97/44065 (Cytotherapeutics). By selecting a packaging cell line capable of producing lentiviral particles, transduction of non-dividing cells in the therapeutic area is obtained. By using retroviral particles capable of transducing only dividing cells, transduction is restricted to de-novo differentiated cells in the therapeutic area.

VI. Target Tissues for Treatment of Neurodegenerative Disorders

An important parameter is the selection of a suitable target tissue. A region of the brain is selected for its retained responsiveness to Neublasmin. In humans, CNS neurons, which retain responsiveness to neurotrophic factors into adulthood include the cholinergic basal forebrain neurons, the entorhinal cortical neurons, the thalamic neurons, the locus coeruleus neurons, the spinal sensory neurons and the spinal motor neurons. Based on the expression pattern, it is expected that cells retaining responsiveness to Neublasmin are found at least in those regions of the brain, in which expression continues into adulthood. In the testes, the target tissue may comprise testicular cancer cells and germ cells.

Abnormalities within the cholinergic compartment of this complex network of neurons have been implicated in a number of neurodegenerative disorders, including AD, Parkinson's disease, and amyotrophic lateral sclerosis (ALS, also known as Lou Gehrig's disease). The cholinergic basal forebrain (particularly, the Ch4 region of the basal forebrain) is a particularly suitable target tissue.

Within the primate forebrain, magnocellular neurons Ch1-Ch4 provide cholinergic innervation to the cerebral cortex, thalamus and basolateral nucleus of the amygdala. In subjects with neurodegenerative diseases such as AD, neurons in the Ch4 region (nucleus basalis of Meynert) which have nerve growth factor (NGF) receptors undergo marked atrophy as compared to normal controls (see, e.g., Kobayashi, et al., Mol. Chem. Neuropathol., 15: 193-206 (1991)).

In normal subjects, neurotrophins prevent sympathetic and sensory neuronal death during development and prevents cholinergic neuronal degeneration in adult rats and primates (Tuszynski, et al., Gene Therapy, 3: 305314 (1996)). The resulting loss of functioning neurons in this region of the basal forebrain is believed to be causatively linked to the cognitive decline experienced by subjects suffering from neurodegenerative conditions such as AD (Tuszynski, et al., supra and, Lehericy, et al., J. Comp. Neurol., 330: 15-31 (1993)).

In human AD, basal forebrain neuronal loss occurs over an intraparenchymal area of approximately 1 cm in diameter. To treat affected neurons over such a large region, treatment with Neublasmin protein formulation or vector composition at upwards of 10 separate in vivo gene vector delivery sites is desirable. However, in treating localized injuries to the basal forebrain, the affected areas of the brain will likely be smaller such that selection of fewer delivery sites (e.g., 5 or fewer) will be sufficient for restoration of a clinically significant number of cholinergic neurons.

In a preferred embodiment, the administration site is the striatum of the brain, in particular the caudate putamen. Injection into the putamen can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra.

Importantly, specific in vivo gene delivery sites are selected so as to cluster in an area of neuronal loss. Such areas may be identified clinically using a number of known techniques, including magnetic resonance imaging (MRI) and biopsy. In humans, non-invasive, in vivo imaging methods such as MRI will be preferred. Once areas of neuronal loss are identified, delivery sites are selected for stereotaxic distribution so each unit dosage of Neublasmin is delivered into the brain at, or within 500 μm from, a targeted cell, and no more than about 10 mm from another delivery site.

VII. Dosing Requirements and Delivery Protocol for Gene Therapy

A further important parameter is the dosage of Neublasmin to be delivered into the target tissue. In this regard, “unit dosage” refers generally to the concentration of Neublasmin/ml of Neublasmin composition. For viral vectors, the Neublasmin concentration may be defined by the number of viral particles/ml of Neublasmin composition. Optimally, for delivery of Neublasmin using a viral expression vector, each unit dosage of Neublasmin will comprise 2.5 to 25 μL of a Neublasmin composition, wherein the composition includes a viral expression vector in a pharmaceutically acceptable fluid and provides from 10¹⁰ up to 10¹⁵ Neublasmin expressing viral particles per ml of Neublasmin composition. Such high titers are particularly suited for Adeno-associated virus.

Within a given target site, the vector system may transduce a target cell. The target cell may be a cell found in nervous tissue, such as a neuron, astrocyte, oligodendrocyte, microglia or ependymal cell. In a preferred embodiment, the target cell is a neuron, in particular a TH positive neuron.

The vector system is preferably administered by direct injection. Methods for injection into the brain (in particular the striatum) are well known in the art (Bilang-Bleuel et al (1997) Proc. Acad. Natl. Sci. USA 94:8818-8823; Choi-Lundberg et al (1998) Exp. Neurol. 154:261-275; Choi-Lundberg et al (1997) Science 275:838-841; and Mandel et al (1997)) Proc. Acad. Natl. Sci. USA 94:14083-14088). Stereotaxic injections may be given.

As mentioned above, for transduction in tissues such as the brain, it is necessary to use very small volumes, so the viral preparation is concentrated by ultracentrifugation. The resulting preparation should have at least 10⁸ t.u./ml, preferably from 10⁸ to 10¹⁰ t.u./ml, more preferably at least 10⁹ t.u./ml. (The titer is expressed in transducing units per ml (t.u./ml) as described in example 7). These titers are typical for lentivirus. It has been found that improved dispersion of transgene expression can be obtained by increasing the number of injection sites and decreasing the rate of injection (Horellou and Mallet (1997) as above). Usually between 1 and 10 injection sites are used, more commonly between 2 and 6. For a dose comprising 1−5×10⁹ t.u./ml, the rate of injection is commonly between 0.1 and 10 μl/min, usually about 1 μl/min.

The Neublasmin composition is delivered to each delivery cell site in the target tissue by microinjection, infusion, scrape loading, electroporation or other means suitable to directly deliver the composition directly into the delivery site tissue through a surgical incision. The delivery is accomplished slowly, such as over a period of about 5-10 minutes (depending on the total volume of Neublasmin composition to be delivered).

Those of skill in the art will appreciate that the direct delivery method employed by the invention obviates a limiting risk factor associated with in vivo gene therapy; to wit, the potential for transduction of non-targeted cells with the vector carrying the Neublasmin encoding transgene. In the invention, delivery is direct and the delivery sites are chosen so diffusion of secreted Neublasmin takes place over a controlled and pre-determined region of the brain to optimise contact with targeted neurons, while minimizing contact with non-targeted cells.

VIII. Predictive Medicine and Diagnostic Uses

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining Neublasmin protein and/or nucleic acid expression as well as Neublasmin activity, in the context of a biological sample (e.g., blood, serum, semen, cells, tissue) to thereby determine whether an individual is afflicted with a disease or disorder, or is at risk of developing a disorder, associated with aberrant Neublasmin expression or activity, such as testicle cancer or male infertility. The invention also provides for prognostic (or predictive) assays for determining whether an individual is at risk of developing a disorder associated with Neublasmin protein, nucleic acid expression or activity (e.g. testicular germ cell tumours, TGCT). For example, mutations in a Neublasmin gene can be assayed in a biological sample. Such assays can be used for prognostic or predictive purpose to thereby prophylactically treat an individual prior to the onset of a disorder characterized by or associated with Neublasmin protein, nucleic acid expression, or biological activity.

Another aspect of the invention provides methods for determining Neublasmin protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of Neublasmin in clinical trials.

These and other agents are described in further detail in the following sections.

Diagnostic Assays

An exemplary method for detecting the presence or absence of Neublasmin in a biological sample involves obtaining a biological sample from a test subject and contacting the biological sample with a compound or an agent capable of detecting Neublasmin protein or nucleic acid (e.g., mRNA, genomic DNA) that encodes Neublasmin protein such that the presence of Neublasmin is detected in the biological sample. An agent for detecting Neublasmin mRNA or genomic DNA is a labeled nucleic acid probe capable of hybridizing to Neublasmin mRNA or genomic DNA. The nucleic acid probe can be, for example, a full-length Neublasmin nucleic acid, such as the nucleic acid of SEQ ID NOS:1, 3, or 6, or a portion thereof, such as an oligonucleotide of at least 15, 30, 50, 100, 250 or 500 nucleotides in length and sufficient to specifically hybridize under stringent conditions to Neublasmin mRNA or genomic DNA, preferably to Neublasmin mRNA. Other suitable probes for use in the diagnostic assays of the invention are described herein.

An agent for detecting Neublasmin protein is an antibody capable of binding to Neublasmin protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect Neublasmin mRNA, protein, or genomic DNA in a biological sample in vitro as well as in vivo. For example, in vitro techniques for detection of Neublasmin mRNA include Northern hybridizations and in situ hybridizations. In vitro techniques for detection of Neublasmin protein include enzyme linked immunosorbent assays (ELISAs), Radio Immuno Assays (RIA), Western blots, immunoprecipitations, and immunofluorescence. In vitro techniques for detection of Neublasmin genomic DNA include Southern hybridizations. Furthermore, in vivo techniques for detection of Neublasmin protein include introducing into a subject a labeled anti-Neublasmin antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject. Another preferred biological sample is a sperm sample.

Diagnostic Applications of Antibodies Directed Against the Proteins of the Invention

Antibodies directed against a Neublasmin protein may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

ELISA Assay

One agent for detecting Neublasmin protein is an antibody capable of binding to a Neublasmin protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is semen, sperm, blood and a fraction or component of blood including blood serum, blood plasma, or lymph. Preferably, the detection of Neublasmin polypeptide is performed on an isolated blood or sperm sample.

That is, the detection method of the invention can be used to detect a Neublasmin protein in a biological sample in vitro as well as in vivo. In vitro techniques for detection of a Neublasmin protein include enzyme linked immunosorbent assays (ELISAs), Radio Immuno Assays (RIAs), Western blots, immunoprecipitations, and immunofluorescence. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-Neublasmin antibody. For example, the antibody can be labeled with a radioactive marker whose presence and location in a subject can be detected by standard imaging techniques.

Enzyme-linked Immunosorbant Assays (ELISAs) combine the specificity of antibodies with the sensitivity of simple enzyme assays, by using antibodies coupled to an easily-assayed enzyme that possesses a high turnover number. ELISAs can provide a useful measurement of antigen.

One of the most useful of the immunoassays is the two-antibody “sandwich” ELISA. This assay is used to determine the antigen concentration in unknown samples. This ELISA is fast and accurate, the assay can determine the absolute amounts of antigen in unknown samples by using a purified antigen standard. The sandwich ELISA requires two antibodies that bind to epitopes that do not overlap on the antigen. This can be accomplished with either two monoclonal antibodies that recognize discrete sites or one batch of affinity-purified polyclonal antibodies.

To utilize this assay, one antibody (the “capture” antibody) is purified and bound to a solid phase. Antigen is then added and allowed to complex with the bound antibody. Unbound products are then removed with a wash, and a labeled second antibody (the “detection” antibody) is allowed to bind to the antigen, thus completing the “sandwich”. The assay is then quantitated by measuring the amount of labeled second antibody bound to the matrix, through the use of a calorimetric substrate. A major advantage of this technique is that the antigen does not need to be purified prior to use, also that these assays are very specific. However, one disadvantage is that not all antibodies can be used. Monoclonal antibody combinations must be qualified at “matched pairs”, meaning that they can recognize separate epitopes on the antigen.

ELISA procedures utilize substrates that produce soluble products. Popular enzymes are those which convert a colorless substrate to a colored product, e.g. p-nitrophenylphosphate (pNPP) which is converted to the yellow p-nitrophenol by alkaline phosphatase. Substrates used with peroxidase include 2,2¢-azo-bis(3-ethylbenzthiazoline-6-sulfonic acid) (ABTS), o-phenylenediamine (OPD) and 3,3¢5,5¢-tetramethylbenzidine base (TMB), which yield green orange and blue colors, respectively.

The sensitivity of the Sandwich ELISA is dependent on four factors:

1. The number of molecules of the first antibody that are bound to the solid phase.

2. The avidity of the first antibody for the antigen.

3. The avidity of the second antibody for the antigen.

4. The specific activity of the second antibody.

The amount of the capture antibody that is bound to the solid phase can be adjusted easily by dilution or concentration of the antibody solution. The avidity of the antibodies for the antigen can only be altered by substitution with other antibodies. The specific activity of the second antibody is determined by the number and type of labeled moieties it contains. Antibodies can be labeled conveniently with iodine, enzymes, or biotin.

General Protocol for the Sandwich ELISA Method:

1. Before the assay, both antibody preparations should be purified and one must be labeled.

2. For most applications, a polyvinylchloride (PVC) microtiter plate is best; however, consult manufacturer guidelines to determine the most appropriate type of plate for protein binding.

3. Bind the unlabeled antibody to the bottom of each well by adding approximately 50 mL of antibody solution to each well (20 mg/mL in PBS). PVC will bind approximately 100 ng/well (300 ng/cm²). The amount of antibody used will depend on the individual assay, but if maximal binding is required, use at least 1 m g/well. This is well above the capacity of the well, but the binding will occur more rapidly, and the binding solution can be saved and used again.

4. Incubate the plate overnight at 4° C. to allow complete binding.

5. Wash the wells twice with PBS. A 500 mL squirt bottle is convenient. The antibody solution washes can be removed by flicking the plate over a suitable container.

6. The remaining sites for protein binding on the microtiter plate must be saturated by incubating with blocking buffer. Fill the wells to the top with 3% BSA/PBS with 0.02% sodium azide. Incubate for 2 hrs to overnight in a humid atmosphere at room temperature. (Note: Sodium azide is an inhibitor or horseradish peroxidase. Do not include sodium azide in buffers or wash solutions, if an HRP-labeled antibody will be used for detection.)

7. Wash wells twice with PBS.

8. Add 50 mL of the antigen solution to the wells (the antigen solution should be titrated). All dilutions should be done in the blocking buffer (3% BSA/PBS with 0.02% sodium azide). Incubate for at least 2 hrs at room temperature in a humid atmosphere.

9. Wash the plate four times with PBS.

10. Add the labeled second antibody. The amount to be added can be determined in preliminary experiments. For accurate quantitation, the second antibody should be used in excess. All dilutions should be done in the blocking buffer.

11. Incubate for 2 hrs or more at room temperature in a humid atmosphere.

12. Wash with several changes of PBS.

13. Add substrate as indicated by manufacturer. After suggested incubation time has elapsed, optical densities at target wavelengths can be measured on an ELISA reader. For quantitative results, the signal of unknown samples is compared against those of a standard curve.

Radioimmunoassay was the first method used to measure minute quantities of biological substances with the help of radioactively labelled molecules.

Radioimmunoassays are based on the reaction between an antibody and an antigen whose concentration has to be quantified. There are several ways to quantify the antigen concentration but the most frequently used method is the indirect assay. In this assay a known quantity of radioactively labelled antigen is mixed with a dilution series of “cold” antigen. The dilution series is brought to reaction with a fixed amount of antibody specific against the antigen. Since cold and radioactively labelled antigens compete with each other for the antibody binding sites, a high concentration of antigen will result in little radioactive antigen bound to the antibody and vice versa. After a fixed time, a second antibody directed against the first antibody is used which leads to the formation of large complexes which upon centrifugation are counted with a radioactive counter. This fraction contains the “cold” and the radioactive antigen which has bound to the specific antibody, while the supernatant in the centrifugate contains the unbound antigen. The serially diluted probes yield points on a curve relating radioactive counts to the concentration of cold antigen: a so-called (cold) reference curve. Using this reference curve, an unknown quantity of antigen in a probe can be quantified by performing the same reactions with first specific, then unspecific antibody and a fixed amount of radioactive antigen. Identification of the radioactive counts in the centrifugate and use of the reference curve yields the unknown antigen concentration.

Radioimmunoassays are very sensitive, are able to detect nano- or picomoles of molecules and have provided a large amount of information on the biochemical processes dealing with ligand-receptor systems.

In another embodiment, the methods further involve obtaining a control biological sample from a control subject, contacting the control sample with a compound or agent capable of detecting Neublasmin protein, mRNA, or genomic DNA, such that the presence of Neublasmin protein, mRNA or genomic DNA is detected in the biological sample, and comparing the presence of Neublasmin protein, mRNA or genomic DNA in the control sample with the presence of Neublasmin protein, mRNA or genomic DNA in the test sample. Preferably, Neublasmin protein or mRNA is detected.

The invention also encompasses kits for detecting the presence of Neublasmin in a biological sample. For example, the kit can comprise: a labeled compound or agent capable of detecting Neublasmin protein or mRNA in a biological sample; means for determining the amount of Neublasmin in the sample; and means for comparing the amount of Neublasmin in the sample with a standard. The compound or agent can be packaged in a suitable container. The kit can further comprise instructions for using the kit to detect Neublasmin protein or nucleic acid.

Prognostic Assays

The diagnostic methods described herein can furthermore be utilized to identify subjects having or at risk of developing a disease or disorder associated with aberrant Neublasmin expression or activity. For example, the assays described herein, such as the preceding diagnostic assays or the following assays, can be utilized to identify a subject having or at risk of developing a disorder associated with Neublasmin protein, nucleic acid expression or activity. Alternatively, the prognostic assays can be utilized to identify a subject having or at risk for developing a disease or disorder. Thus, the invention provides a method for identifying a disease or disorder associated with aberrant Neublasmin expression or activity in which a test sample is obtained from a subject and Neublasmin protein or nucleic acid (e.g., mRNA, genomic DNA, preferably mRNA) is detected, wherein the presence and/or amount of Neublasmin protein or nucleic acid is diagnostic for a subject having or at risk of developing a disease or disorder associated with aberrant Neublasmin expression or activity. As used herein, a “test sample” refers to a biological sample obtained from a subject of interest. For example, a test sample can be a biological fluid (e.g., serum, or semen), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant Neublasmin expression or activity. For example, such methods can be used to determine whether a subject can be effectively treated with an agent for a disorder. Thus, the invention provides methods for determining whether a subject can be effectively treated with an agent for a disorder associated with aberrant Neublasmin expression or activity in which a test sample is obtained and Neublasmin protein or nucleic acid is detected (e.g., wherein the presence of Neublasmin protein or nucleic acid is diagnostic for a subject that can be administered the agent to treat a disorder associated with aberrant Neublasmin expression or activity).

The methods of the invention can also be used to detect genetic lesions in a Neublasmin gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a Neublasmin-protein, or the misexpression of the Neublasmin gene. For example, such genetic lesions can be detected by ascertaining the existence of at least one of: (i) a deletion of one or more nucleotides from a Neublasmin gene; (ii) an addition of one or more nucleotides to a Neublasmin gene; (iii) a substitution of one or more nucleotides of a Neublasmin gene, (iv) a chromosomal rearrangement of a Neublasmin gene; (v) an alteration in the level of a messenger RNA transcript of a Neublasmin gene, (vi) aberrant modification of a Neublasmin gene, such as of the methylation pattern of the genomic DNA, (vii) the presence of a non-wild-type splicing pattern of a messenger RNA transcript of a Neublasmin gene, (viii) a non-wild-type level of a Neublasmin protein, (ix) allelic loss of a Neublasmin gene, and (x) inappropriate post-translational modification of a Neublasmin protein. As described herein, there are a large number of assay techniques known in the art which can be used for detecting lesions in a Neublasmin gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the Neublasmin-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a Neublasmin gene under conditions such that hybridization and amplification of the Neublasmin gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a Neublasmin gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in Neublasmin can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in Neublasmin can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the Neublasmin gene and detect mutations by comparing the sequence of the sample Neublasmin with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the Neublasmin gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type Neublasmin sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in Neublasmin cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a Neublasmin sequence, e.g., a wild-type Neublasmin sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in Neublasmin genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control Neublasmin nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265:12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell. Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a Neublasmin gene.

Furthermore, any cell type or tissue, preferably peripheral blood leukocytes, in which Neublasmin is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on Neublasmin activity (e.g., Neublasmin gene expression), as identified by a screening assay described herein can be administered to individuals to treat (prophylactically or therapeutically) disorders. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of Neublasmin protein, expression of Neublasmin nucleic acid, or mutation content of Neublasmin genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome Pregnancy Zone Protein Precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of Neublasmin protein, expression of Neublasmin nucleic acid, or mutation content of Neublasmin genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a Neublasmin modulator, such as a modulator identified by one of the exemplary screening assays described herein.

Monitoring of Effects During Clinical Trials

Monitoring the influence of agents (e.g., drugs, compounds) on the expression or activity of Neublasmin (e.g., the ability to modulate aberrant cell proliferation and/or differentiation) can be applied not only in basic drug screening, but also in clinical trials. For example, the effectiveness of an agent determined by a screening assay as described herein to increase Neublasmin gene expression, protein levels, or upregulate Neublasmin activity, can be monitored in clinical trails of subjects exhibiting decreased Neublasmin gene expression, protein levels, or downregulated Neublasmin activity. Alternatively, the effectiveness of an agent determined by a screening assay to decrease Neublasmin gene expression, protein levels, or downregulate Neublasmin activity, can be monitored in clinical trails of subjects exhibiting increased Neublasmin gene expression, protein levels, or upregulated Neublasmin activity. In such clinical trials, the expression or activity of Neublasmin and, preferably, other genes that have been implicated in, for example, a cellular proliferation or immune disorder can be used as a “read out” or markers of the immune responsiveness of a particular cell.

By way of example, and not of limitation, genes, including Neublasmin, that are modulated in cells by treatment with an agent (e.g., compound, drug or small molecule) that modulates Neublasmin activity (e.g., identified in a screening assay as described herein) can be identified. Thus, to study the effect of agents on cellular proliferation disorders, for example, in a clinical trial, cells can be isolated and RNA prepared and analyzed for the levels of expression of Neublasmin and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of Neublasmin or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a Neublasmin protein, mRNA, or genomic DNA in the preadministration sample; (iii) obtaining one or more post-administration samples from the subject; (iv) detecting the level of expression or activity of the Neublasmin protein, mRNA, or genomic DNA in the post-administration samples; (v) comparing the level of expression or activity of the Neublasmin protein, mRNA, or genomic DNA in the pre-administration sample with the Neublasmin protein, mRNA, or genomic DNA in the post administration sample or samples; and (vi) altering the administration of the agent to the subject accordingly. For example, increased administration of the agent may be desirable to increase the expression or activity of Neublasmin to higher levels than detected, i.e., to increase the effectiveness of the agent. Alternatively, decreased administration of the agent may be desirable to decrease expression or activity of Neublasmin to lower levels than detected, i.e., to decrease the effectiveness of the agent.

IX. Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells, which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV.

A special and preferred type of retroviruses include the lentiviruses which can transduce a cell and integrate into its genome without cell division. Thus preferably the vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Adeno-associated virus can also transducer a cell and integrate into its genome without cell division. Methods for preparation and administration of adeno-associated virus are described in U.S. Pat. No. 5,667,158, U.S. Pat. No. 6,309,634, and U.S. Pat. No. 6,683,058.

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome.

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors. These vectors may be particularly useful in the central nervous system for cancer treatment, where there is a relative lack of cell division in adult patients.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the ψ signal, and expressing the gag/pol genes and the env gene from separate transcriptional units. In addition, in some 2. and 3. generation retroviruses, the 5′LTR's have been replaced with non-viral promoters controlling the expression of these genes, and the 3′ promoter has been minimised to contain only the proximal promoter. These designs minimize the possibility of recombination leading to production of replication competent vectors, or helper viruses. See, e.g., U.S. Pat. No. 4,861,719, herein incorporated by reference.

In one aspect, the invention relates to a packaging cell line capable of producing an infective virus particle, said virus particle comprising a Retroviridae derived genome comprising a 5′ retroviral LTR, a tRNA binding site, a packaging signal, a promoter operably linked to a polynucleotide sequence encoding the polypeptide of the invention, an origin of second strand DNA synthesis, and a 3′ retroviral LTR.

X. Expression Vectors

In one aspect, the invention relates to an expression vector comprising the nucleic acid of the invention. Construction of vectors for recombinant expression of Neublasmin for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982).

Briefly, construction of recombinant expression vectors employs standard ligation techniques. For analysis to confirm correct sequences in vectors constructed, the genes are sequences using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134, 1982).

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, and Elongation Factor 1 alpha promoter (EF1-alpha).

Examples of inducible/repressible promoters include: Tet-On, Tet-Off, Rapamycin-inducible promoter, Mx1.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Woodchuck hepatitis virus post-transcriptional regulation element, WPRE, SP163, rat InsulinII-intron or other introns, CMV enhancer, and Chicken [beta]-globin insulator or other insulators.

Transgene expression may also be increased for long term stable expression using cytokines to modulate promoter activity. Several cytokines have been reported to modulate the expression of transgene from collagen 2 (I) and LTR promoters (Chua et al., connective Tissue Res., 25: 161-170 (1990); Elias et al., Annals N.Y. Acad. Sci., 580: 233-244 (1990)); Seliger et al., J. Immunol. 141: 2138-2144 (1988) and Seliger et al., J. Virology 62: 619-621 (1988)). For example, transforming growth factor (TGF), interleukin (IL)-I, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

Collagen promoter with the collagen enhancer sequence (Coll (E)) may also be used to increase transgene expression by suppressing further any immune response to the vector which may be generated in a treated brain notwithstanding its immune-protected status. In addition, anti-inflammatory agents including steroids, for example dexamethasone, may be administered to the treated host immediately after vector composition delivery and continued, preferably, until any cytokine-mediated inflammatory response subsides. An immunosuppression agent such as cyclosporin may also be administered to reduce the production of interferons, which downregulates LTR promoter and Coll (E) promoter-enhancer, and reduces transgene expression.

The vector may comprise further sequences such as a sequence coding for the Cre-recombinase protein, and LoxP sequences. A further way of ensuring temporary expression of the neublastin is through the use of the Cre-LoxP system which results in the excision of part of the inserted DNA sequence either upon administration of Cre-recombinase to the cells (Daewoong et al, Nature Biotechnology 19:929-933) or by incorporating a gene coding for the recombinase into the virus construct (Pluck, Int J Exp Path, 77:269-278). Incorporating a gene for the recombinase in the virus construct together with the LoxP sites and a structural gene (a neublastin in the present case) often results in expression of the structural gene for a period of approximately five days.

XI. Encapsulation of Cells

In one aspect the invention relates to an implantable cell culture device, the device comprising i) a semipermeable membrane permitting the diffusion of a protein of the invention; and ii) a composition of cells according to the invention. Encapsulated cell therapy is based on the concept of isolating cells from the recipient host's immune system by surrounding the cells with a semipermeable biocompatible material before implantation within the host. The invention includes a device in which cells capable of expressing and secreting Neublasmin are encapsulated in an immunoisolatory capsule. An “immunoisolatory capsule” means that the capsule, upon implantation into a recipient host, minimizes the deleterious effects of the host's immune system on the cells in the core of the device. Cells are immunoisolated from the host by enclosing them within implantable polymeric capsules formed by a microporous membrane. This approach prevents the cell-to cell contact between host and implanted tissues, eliminating antigen recognition through direct presentation. The membranes used can also be tailored to control the diffusion of molecules, such as antibody and complement, based on their molecular weight (Lysaght et al., 56 J. Cell Biochem. 196 (1996), Colton, 14 Trends Biotechnol. 158 (1996)). Using encapsulation techniques, Cells can be transplanted into a host without immune rejection, either with or without use of immunosuppressive drugs. Useful biocompatible polymer capsules usually contain a core that contains cells, either suspended in a liquid medium or immobilized within an immobilizing matrix, and a surrounding or peripheral region of permselective matrix or membrane (“jacket”) that does not contain isolated cells, that is biocompatible, and that is sufficient to protect cells in the core from detrimental immunological attack. Encapsulation hinders elements of the immune system from entering the capsule, thereby protecting the encapsulated cells from immune destruction. The semipermeable nature of the capsule membrane also permits the biologically active molecule of interest to easily diffuse from the capsule into the surrounding host tissue.

The capsule can be made from a biocompatible material. A “biocompatible material” is a material that, after implantation in a host, does not elicit a detrimental host response sufficient to result in the rejection of the capsule or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics. Preferably the capsule of this invention will be similar to those described by PCT International patent applications WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050, incorporated by reference. Such capsules allow for the passage of metabolites, nutrients and therapeutic substances while minimizing the detrimental effects of the host immune system. Components of the biocompatible material may include a surrounding semipermeable membrane and the internal cell-supporting scaffolding. Preferably, the genetically altered cells are seeded onto the scaffolding, which is encapsulated by the permselective membrane. The filamentous cell-supporting scaffold may be made from any biocompatible material selected from the group consisting of acrylic, polyester, polyethylene, polypropylene polyacetonitrile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (WO 98/05304, incorporated by reference). Woven mesh tubes have been used as vascular grafts (WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

The capsule can be any configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

When macrocapsules are used, preferably between 10³ and 10⁸ cells are encapsulated, most preferably 10⁵ to 10⁷ cells are encapsulated in each device. Dosage may be controlled by implanting a fewer or greater number of capsules, preferably between 1 and 10 capsules per patient.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the capsules may be used, including the use of polymer adhesives or crimping, knotting and heat sealing. In addition, any suitable “dry” sealing method can also be used, as described, e.g., in U.S. Pat. No. 5,653,687, incorporated by reference.

The encapsulated cell devices are implanted according to known techniques. Many implantation sites are contemplated for the devices and methods of this invention. These implantation sites include, but are not limited to, the central nervous system, including the brain, spinal cord (see, U.S. Pat. Nos. 5,106,627, 5,156,844, and 5,554,148, incorporated by reference), and the aqueous and vitreous humors of the eye (see, WO 97/34586, incorporated by reference).

The ARPE-19 cell line is a superior platform cell line for encapsulated cell based delivery technology and is also useful for unencapsulated cell based delivery technology. The ARPE-19 cell line is hardy (i.e., the cell line is viable under stringent conditions, such as implantation in the central nervous system or the intra-ocular environment). ARPE-19 cells can be genetically modified to secrete a substance of therapeutic interest. ARPE-19 cells have a relatively long life span. ARPE-19 cells are of human origin. Furthermore, encapsulated ARPE-19 cells have good in vivo device viability. ARPE-19 cells can deliver an efficacious quantity of growth factor. ARPE-19 cells elicit a negligible host immune reaction. Moreover, ARPE-19 cells are non-tumorigenic.

In order to ensure correct processing of the encoded Neublasmin, the encapsulated cells may additionally be transfected or transduced with an expression construct coding for proprotein-convertase 4.

Methods and apparatus for implantation of capsules into the CNS are described in U.S. Pat. No. 5,487,739. Methods and apparatus for implantation of capsules into the eye are described in U.S. Pat. No. 5,904,144, U.S. Pat. No. 6,299,895, U.S. Pat. No. 6,439,427, and US 20030031700.

In one aspect the invention relates to a biocompatible capsule comprising: a core comprising living packaging cells that secrete a viral vector for infection of a target cell, wherein the viral vector is a vector according to the invention; and an external jacket surrounding said core, said jacket comprising a permeable biocompatible material, said material having a porosity selected to permit passage of retroviral vectors of approximately 100 nm diameter thereacross, permitting release of said viral vector from said capsule.

Preferably, the core additionally comprises a matrix, the packaging cells being immobilized by the matrix. According to one embodiment, the jacket comprises a hydrogel or thermoplastic material.

Examples of suitable cells for packaging cell lines include HEK293, NIH3T3, PG13, and ARPE-19 cells. Preferred cells include PG13 and 3T3 cells.

Packaging cell lines may be encapsulated and administered using the methods and compositions disclosed in U.S. Pat. No. 6,027,721 and WO 97/01357 hereby incorporated by reference in their entirety.

XII. Host Cells

In one aspect the invention relates to isolated host cells genetically modified with the vector according to the invention.

According to one embodiment, the host cells are prokaryotic cells such as E. coli which are capable producing recombinant protein in high quantities and which can easily be scaled up to industrial scale. The use of prokaryotic producer cells may require refolding and glycosylation of the Neublasmin in order to obtain a biologically active protein.

According to another embodiment, the cells preferably are mammalian host cells because these are capable of secreting and processing the encoded Neublasmin correctly. Preferred species include the group consisting of rodent (mouse, rat), rabbit, dog, cat, pig, monkey, human being.

Examples of primary cultures and cell lines that are good candidates for transduction or transfection with the vectors of the present invention include the group consisting of CHO, HEK293, COS, PC12, HiB5, RN33b, neuronal cells, foetal cells, ARPE-19, C2C12, HeLa, HepG2, striatal cells, neurons, astrocytes, interneurons.

For in vivo transduction or transfection, the preferred group of host cells include cRET expressing cells; testes cells, such as immature cells in testis; striatal cells, neurons, astrocytes and interneurons. For ex vivo gene therapy, the preferred group of cells include neuronal cells, neuronal precursor cells, neuronal progenitor cells, stem cells and foetal cells. For encapsulation the preferred cells include ARPE-19, and PC12.

XIII. Antibodies.

The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, antigenic peptide fragments of the antigen can be used as immunogens. An antigenic peptide fragment comprises at least 6 amino acid residues of the amino acid sequence of the full length mature protein, such as an amino acid sequence shown in SEQ ID NOs 9, 10, 14, 18, and 25, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions. More preferably the antigenic peptide is a C-terminal or N-terminal peptide of Neublasmin (selected from the group consisting of SEQ ID No.26-35). In one embodiment the antigenic peptide is a C-terminal peptide selected from the group consisting of peptides having the amino acid sequence of SEQ ID No. 28, 29, 31, 33, and 35. More preferably, the C-terminal peptide is a human C-terminal peptide having the amino acid sequence of SEQ ID No. 28. In another embodiment the antigenic peptide is an N-terminal peptide selected from the group consisting of peptides having the amino acid sequence of SEQ ID No. 26, 27, 30, 32, and 34. More preferably the N-terminal peptide is a human N-terminal peptide having the amino acid sequence of SEQ ID No. 26.

Shorter peptides can also be used as antigens to generate antibodies for diagnostic and therapeutic uses. Specific examples include:

QPLRSQRSVPEAFS (SEQ ID No. 36)

QQRKRDGPDLAEYYY (SEQ ID No. 37)

DGPDLAEYYYDAHL (SEQ ID No. 38)

In certain embodiments of the invention, at least one epitope encompassed by the antigenic peptide is a region of Neublasmin that is located on the surface of the protein, e.g., a hydrophilic region. A hydrophobicity analysis of the human Neublasmin protein sequence will indicate which regions of a Neublasmin polypeptide are particularly hydrophilic and, therefore, are likely to constitute surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein ore peptide, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic Neublasmin polypeptide. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof, or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Administration of the antibody may abrogate or inhibit the binding of the target with an endogenous receptor to which it naturally binds. In this case, the antibody binds to the target and masks a binding site of the naturally occurring receptor. Thus the receptor cannot mediate the signal transduction pathway for which ligand is normally responsible.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume of the subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

XIV. Screening and Detection Methods

The isolated nucleic acid molecules of the invention can be used to express Neublasmin protein (e.g., via a recombinant expression vector in a host cell in gene therapy applications), to detect Neublasmin mRNA (e.g., in a biological sample) or a genetic lesion in a Neublasmin gene, and to modulate Neublasmin activity, as described further, below. In addition, the Neublasmin proteins can be used to screen drugs or compounds that modulate the Neublasmin protein activity or expression as well as to treat disorders characterized by insufficient or excessive production of Neublasmin protein or production of Neublasmin protein forms that have decreased or aberrant activity compared to Neublasmin wild-type protein. In addition, the anti-Neublasmin antibodies of the invention can be used to detect and isolate Neublasmin proteins and modulate Neublasmin activity.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to Neublasmin proteins or have a stimulatory or inhibitory effect on, e.g., Neublasmin protein expression or Neublasmin protein activity. The invention also includes compounds identified in the screening assays described herein.

In one embodiment, the invention provides assays for screening candidate or test compounds which bind to or modulate the activity of a Neublasmin protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a compound that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-free assay in which Neublasmin protein, or a biologically-active portion thereof, is contacted with a test compound and the ability of the test compound to bind to a Neublasmin protein determined. Determining the ability of the test compound to bind to the Neublasmin protein can be accomplished, for example, by coupling the test compound with a radioisotope or enzymatic label such that binding of the test compound to the Neublasmin protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting Neublasmin protein, or a biologically-active portion thereof, with a known compound which binds Neublasmin to form an assay mixture, contacting the assay mixture with a test compound, and determining the ability of the test compound to interact with a Neublasmin protein, wherein determining the ability of the test compound to interact with a Neublasmin protein comprises determining the ability of the test compound to preferentially bind to Neublasmin protein or a biologically-active portion thereof as compared to the known compound.

In another embodiment, an assay comprises contacting a Neublasmin protein, or a biologically-active portion thereof, with a test compound and determining the ability of the test compound to modulate (e.g., stimulate or inhibit) the activity of the Neublasmin protein or biologically-active portion thereof. Determining the ability of the test compound to modulate the activity of Neublasmin or a biologically-active portion thereof can be accomplished, for example, by determining the ability of the Neublasmin protein to bind to or interact with a Neublasmin target molecule. As used herein, a “target molecule” is a molecule with which a Neublasmin protein binds or interacts in nature, for example, a molecule on the surface of a cell which expresses a Neublasmin interacting protein, a molecule on the surface of a second cell, a molecule in the extracellular milieu, a molecule associated with the internal surface of a cell membrane or a cytoplasmic molecule. A Neublasmin target molecule can be a non-Neublasmin molecule or a Neublasmin protein or polypeptide of the invention. In one embodiment, a Neublasmin target molecule is a component of a signal transduction pathway that facilitates transduction of an extracellular signal through the cell membrane and into the cell. The target, for example, can be an intercellular protein that has catalytic activity or a protein that facilitates the association of downstream signaling molecules with Neublasmin.

Determining the ability of the Neublasmin protein to bind to or interact with a Neublasmin target molecule can be accomplished by one of the methods described above for determining direct binding. In one embodiment, determining the ability of the Neublasmin protein to bind to or interact with a Neublasmin target molecule can be accomplished by determining the activity of the target molecule. For example, the activity of the target molecule can be determined by detecting induction of a cellular second messenger of the target (i.e. intracellular Ca²⁺ diacylglycerol, IP₃, etc.), detecting catalytic/enzymatic activity of the target an appropriate substrate, detecting the induction of a reporter gene (comprising a Neublasmin-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a Neublasmin protein or biologically-active portion thereof with a test compound and determining the ability of the test compound to bind to the Neublasmin protein or biologically-active portion thereof. Binding of the test compound to the Neublasmin protein can be determined either directly or indirectly as described above. In one such embodiment, the assay comprises contacting the Neublasmin protein or biologically-active portion thereof with a known compound which binds Neublasmin to form an assay mixture, contacting the assay mixture with a test compound, and determining the ability of the test compound to interact with a Neublasmin protein, wherein determining the ability of the test compound to interact with a Neublasmin protein comprises determining the ability of the test compound to preferentially bind to Neublasmin or biologically-active portion thereof as compared to the known compound.

In still another embodiment, an assay is a cell-free assay comprising contacting Neublasmin protein or biologically-active portion thereof with a test compound and determining the ability of the test compound to modulate (e.g. stimulate or inhibit) the activity of the Neublasmin protein or biologically-active portion thereof. Determining the ability of the test compound to modulate the activity of Neublasmin can be accomplished, for example, by determining the ability of the Neublasmin protein to bind to a Neublasmin target molecule by one of the methods described above for determining direct binding. In an alternative embodiment, determining the ability of the test compound to modulate the activity of Neublasmin protein can be accomplished by determining the ability of the Neublasmin protein further modulate a Neublasmin target molecule. For example, the catalytic/enzymatic activity of the target molecule on an appropriate substrate can be determined as described, supra.

In yet another embodiment, the cell-free assay comprises contacting the Neublasmin protein or biologically-active portion thereof with a known compound which binds Neublasmin protein to form an assay mixture, contacting the assay mixture with a test compound, and determining the ability of the test compound to interact with a Neublasmin protein, wherein determining the ability of the test compound to interact with a Neublasmin protein comprises determining the ability of the Neublasmin protein to preferentially bind to or modulate the activity of a Neublasmin target molecule.

In the case of cell-free assays, it may be desirable to utilize a solubilizing agent to facilitate or enhance maintenance of Neublasmin protein in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethyl-amminiol-1-propane sulfonate (CHAPS), or 3-(3-cholamidopropyl)dimethyl-amminiol-2-hydroxy-1-propane sulfonate (CHAPSO).

In more than one embodiment of the above assay methods of the invention, it may be desirable to immobilize either Neublasmin protein or its target molecule to facilitate separation of complexed from uncomplexed forms of one or both of the proteins, as well as to accommodate automation of the assay. Binding of a test compound to Neublasmin protein, or interaction of Neublasmin protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-Neublasmin fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or Neublasmin protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of Neublasmin protein binding or activity determined using standard techniques.

Other techniques for immobilizing proteins on matrices can also be used in the screening assays of the invention. For example, either the Neublasmin protein or its target molecule can be immobilized utilizing conjugation of biotin and streptavidin. Biotinylated Neublasmin protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with Neublasmin protein or target molecules, but which do not interfere with binding of the Neublasmin protein to its target molecule, can be derivatized to the wells of the plate, and unbound target or Neublasmin protein trapped in the wells by antibody conjugation. Methods for detecting such complexes, in addition to those described above for the GST-immobilized complexes, include immunodetection of complexes using antibodies reactive with the Neublasmin protein or target molecule, as well as enzyme-linked assays that rely on detecting an enzymatic activity associated with the Neublasmin protein or target molecule.

In another embodiment, modulators of Neublasmin protein expression are identified in a method wherein a cell is contacted with a candidate compound and the expression of Neublasmin mRNA or protein in the cell is determined. The level of expression of Neublasmin mRNA or protein in the presence of the candidate compound is compared to the level of expression of Neublasmin mRNA or protein in the absence of the candidate compound. The candidate compound can then be identified as a modulator of Neublasmin mRNA or protein expression based upon this comparison. For example, when expression of Neublasmin mRNA or protein is greater (i.e., statistically significantly greater) in the presence of the candidate compound than in its absence, the candidate compound is identified as a stimulator of Neublasmin mRNA or protein expression. Alternatively, when expression of Neublasmin mRNA or protein is less (statistically significantly less) in the presence of the candidate compound than in its absence, the candidate compound is identified as an inhibitor of Neublasmin mRNA or protein expression. The level of Neublasmin mRNA or protein expression in the cells can be determined by methods described herein for detecting Neublasmin mRNA or protein.

In yet another aspect of the invention, the Neublasmin proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with Neublasmin (“Neublasmin-binding proteins” or “Neublasmin-bp”) and modulate Neublasmin activity. Such Neublasmin-binding proteins are also likely to be involved in the propagation of signals by the Neublasmin proteins as, for example, upstream or downstream elements of the Neublasmin pathway.

The two-hybrid system is based on the modular nature of most transcription factors, which consist of separable DNA-binding and activation domains. Briefly, the assay utilizes two different DNA constructs. In one construct, the gene that codes for Neublasmin is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a Neublasmin-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with Neublasmin.

The invention further pertains to novel agents identified by the aforementioned screening assays and uses thereof for treatments as described herein.

### EXAMPLES

### Example 1

### Obtaining a Full Length Coding Sequence

Human Neublasmin cDNA is available from the IMAGE consortium with the following information:

| | |
|---|---|
| IDENTIFIERS | |
| dbEST Id: | 1034596 |
| EST name: | zt74c05.r1 |
| GenBank Acc: | AA393247 |
| GenBank gi: | 2046232 |
| GDB Id: | 5924984 |
| CLONE INFO | |
| Clone Id: | IMAGE: 728072 (5′) |
| Source: | IMAGE Consortium, LLNL |
| Insert length: | 421 |
| DNA type: | cDNA |
| SEQUENCE | |
| | AGCATAGGGACGACAGGAGCCAGGACCCGTGTAGG |
| | AGTTGGTTCTCGCCATGCTGGGGGCTCTGCACCCC |
| | CGGGCTGGGCTCAGCCTCTTCCTCCACCTCATCCT |
| | GGCAGTGGCACTGGTTCGCTCCCAGCCTCTGAGGT |
| | CTCAGCGGTCTGTTCCTGAGGCATTTTCCGCCCCC |
| | CTGGAACTCTCGCAGCCACTTTCCGGCCTGGTGGA |
| | TGACTATGGCATCCTCCCCAAGCACCCAAGGCCGC |
| | GAGGGCCTCGACCCCTCCTGTCTAGGGCCCAGCAG |
| | CGCAACGGGGNACGGGCCCGACGCTTGCCTGAGTA |
| | TTACGTATGATGCACACCGTATGACNCGCAAGGCC |
| | CGTCATAAAGATACCATGGTGTGACAAAAAAAAAA |
| Quality: | High quality sequence stops at |
| | base: 371 |
| Entry Created: | Apr. 24 1997 |
| Lat Updated: | Aug. 12 1997 |
| COMMENTS | |
| | This clone is available royalty- |
| | free through LLNL; contact the IMAGE |
| | Consortium (info@image.llnl.gov) |
| | for further information. |
| LIBRARY | |
| Lib Name: | Soares_testis_NHT |
| Organism: | Homo sapiens |
| Sex: | male |
| Lab host: | DH10B |
| Vector: | pT7T3D-Pac (Pharmacia) with a |
| | modified polylinker |
| R. Site 1: | Not I |
| R. Site 2: | Eco RI |
| Description: | 1st strand cDNA was prepared from |
| | mRNA obtained from Clontech |
| | Laboratories, Inc., and primed with |
| | a Not I-oligo (dT) primer |
| | [5′ TGTTACCAATCTGAAGTGGGAGCGGCCGCCC |
| | AATTTTTTTTTTTTTTTTT 3′]. |
| | Double-stranded cDNA was ligated to |
| | Eco RI adaptors (Pharmacia), |
| | digested with Not I and cloned into |
| | the Not I and Eco RI sites of the |
| | modified pT7T3 vector. Library went |
| | through one round of normalization |
| | to Cot5, and was constructed by |
| | Bento Soares and M. Fatima Bonaldo. |
| SUBMITTER | |
| Name: | Wilson RK |
| Institution: | Washington University School of |
| | Medicine |
| Address: | 4444 Forest Park Parkway, |
| | Box 8501, St. Louis, MO 63108 |
| Tel: | 314 286 1800 |
| Fax: | 314 286 1810 |
| E-mail: | est@watson.wustl.edu |
| CITATIONS | |
| Title: | WashU-Merck EST Project 1997 |
| Authors: | Hillier, L., Allen, M., Bowles, L., |
| | Dubuque, T., Geisel, G., Jost, S., |
| | Kucaba, T., Lacy, M., Le, N., |
| | Lennon, G., Marra, M., Martin, J., |
| | Moore, B., Schellenberg, K., |
| | Steptoe, M., Tan, F., Theising, B., |
| | White, Y., Wylie, T., Waterston, |
| | R., Wilson, R. |
| Year: | 1997 |
| Status: | Unpublished |

Map Data

The use of IMAGE clones:

Reference that should be used:

You will include the unique and specific identifier of each arrayed clone which was initially assigned by Lawrence Livermore National Laboratory, Livermore, Calif., and accompanies the IMAGE MATERIALS) in data pertaining to the IMAGE MATERIALS submitted to public databases and in resulting publications. This nomenclature consists of the term “I.M.A.G.E. Consortium CloneID” followed by a five to seven digit number. You will refer publicly (including but not limited to electronic and print versions of articles and databases) to these arrayed cDNA clones as the “I.M.A.G.E. Consortium [LLNL] cDNA Clones”, and will reference the following publication: “The I.M.A.G.E. Consortium: An Integrated Molecular Analysis of Genomes and their Expression,” Lennon, G. G., Auffray, C., Polymeropoulos, M., and Soares, M. B. [1996] Genomics 33, pgs. 151-152. In INTERNET/World Wide Web publications and databases, you agree to provide electronic referencing (e.g. ‘anchors’ and/or ‘hotlinks’) to the I.M.A.G.E. Consortium home page, currently located at URL http://image.llnl.gov.

### Example 1b

### Generation of Neublasmin Expression Construct

Neublasmin cDNA is PCR amplified from testis cDNA library using the following primers:

| | |
|---|---|
| 5′ primer: | 5′-CGGGATCCAGGACCCGTGTAGGAGATGG-3′ |
| 3′ primer: | 5′-ATATCTCGAGGGCCTTGGGTCATAGGTGTG-3′ |

PCR reactions is set up with cDNA derived from human testis total RNA (Clontech# cat#64101-1) as DNA template. A proofreading polymerase (pfu-turbo polymerase, Stratagene) is applied for the PCR amplification, with the following amplification profile: pre-denaturation step: 95° C., 1′ followed by 35 3-step cycles: denaturation step: 96° C., 30″; annealing step: 64° C., 30″; elongation step: 72° C., 30″. Then a post-elongation step: 72° C., 2′ followed by cooling to 4° C.

PCR reactions are pooled and the 340 bp Neublasmin PCR fragment is agarose gel-purified and cut with BamHI and XhoI. The BamHI/XhoI-restricted Neublasmin PCR fragment is gel-purified. Five μg of a lentiviral transfer vector, pHsCXW, (GenBank accession #: AY468-486) is digested with BamHI and XhoI and the vector backbone is gel purified.

The BamHI/XhoI Neublasmin PCR fragment is ligated into the BamHI and XhoI sites of the pHsCXW lentiviral transfer vector followed by transformation into XL1-B electrocompetent cells.

### Example 2

### Neublasmin Sequences

cDNA sequences for human, mouse, and rat Neublasmin can be found in Genbank under the accession numbers given below. The Chimpanzee cDNA was constructed by searching with human Neublasmin protein sequence against the Chimpanzee genome by use of the BLAT server (http://genome.ucsc.edu/cgi-bin/hgBlat). Results from the search was used to manually identify donor and acceptor splice site whereafter cDNA was assembled.

Nucleotide Variants:

| Human Neublasmin variant 1 genomic nucleotide sequence | |
|---|---|
| (SEQ ID NO 1) | |
| gagactgctg gccctgcccc agggcaggtg ctgacgcagg cttggaatga | 45960493 |
| aggccctttg tgaggtggcc ctgggagccg gcaacgggtt ccgtcctgcc | 45960443 |
| AGCATAGGGA CGACAGGAGC CAGGACCCGT GTAGGAGATG GTTCTCGCCA | 45960393 |
| TGCTGGGGGC TCTGCACCCC CGGGCTGGGC TCAGCCTCTT CCTCCACCTC | 45960343 |
| ATCCTGGCAG TGGCACTGCT TCGCTCCCAG CCTCTGAGgt gactcctggg | 45960293 |
| gcacagaggg cagggcactt gggccggatg gtggaggtgc tgcccctccc | 45960243 |
| ctctctggag tggggctggc cctggtaccc tgggcgttcg agggcttgcc | 45960193 |
| aagtgccccc acactgagcc atccccccat tcacaaggag gtggcggtgg | 45960143 |
| ccttctgggc cagggctgag caccccgaca tctcccacag GTCTCAGCGG | 45960093 |
| TCTGTTCCTG AGGCATTTTC CGCCCCCCTG GAACTCTCGC AGCCACTTTC | 45960043 |
| CGGCCTGGTG GATGgtagta tctctcttgt ccccttcccc tacccatccc | 45959993 |
| tgacagtgga gacgccccta agaggtgccc caagtccagc cttcaggggc | 45959943 |
| ttgtcatccc aaaccacatc tgttcaaggg tgacttcagt tccatcagag | 45959893 |
| ccagctgccc cctaccaccc atgccctctt ttctctcttt agACTATGGC | 45959843 |
| ATCCTCCCCA AGCACCCAAG GCCGCGAGGG CCTCGACCCC TCCTGTCTAG | 45959793 |
| GGCCCAGCAG CGCAAGCGGG ACGGGCCCGA CCTTGCCGAG TATTACTATG | 45959743 |
| ATGCACACCT ATGACCCAAG GCCCTCATAA AGATACCATG tgtgacaaag | 45959693 |
| gcttggcttt cctgggctgg gagcgagacg gggacgggag gggcccatgc | 45959643 |
| agatacatcc actgcagtcc agacaggaaa tggcacttta | |

Human Neublasmin variant 1 cDNA (360 bp; CDS = 50-334)
(SEQ ID NO 3)

AGCATAGGGACGACAGGAGCCAGGACCCGTGTAGGAGATGGTTCTCGCCATGCTGGGGGCTC
TGCACCCCCGGGCTGGGCTCAGCCTCTTCCTCCACCTCATCCTGGCAGTGGCACTGCTTCGC
TCCCAGCCTCTGAGGTCTCAGCGGTCTGTTCCTGAGGCATTTTCCGCCCCCCTGGAACTCTC
GCAGCCACTTTCCGGCCTGGTGGATGACTATGGCATCCTCCCCAAGCACCCAAGGCCGCGAG
GGCCTCGACCCCTCCTGTCTAGGGCCCAGCAGCGCAAGCGGGACGGGCCCGACCTTGCCGAG
TATTACTATGATGCACACCTATGACCCAAGGCCCTCATAAAGATACCATG
Human Neublasmin variant 2 cDNA (360 bp; CDS = 50-334)

| (SEQ ID NO 3) | |
|---|---|
| AGCATAGGGACGACAGGAGCCAGGACCCGTGTAGGAGATGGTTCTCGCCATGCTGGGGGCTC | |
| TGCACCCCCGGGCTGGGCTCAGCCTCTTCCTCCACCTCATCCTGGCAGTGGCACTGCTTCGC | |
| TCCCAGCCTCTGAGGTCTCAGCGGTCTGTTCCTGAGGCATTTTCCGCCCCCCTGGAACTCTC | |
| GCAGCCACTTTCCGGCCTGGTGGATGACTATGGCATCCTCCCCAAGCACCCAAGGCCGCGAG | |
| GGCCTCGACCCCTCCTGTCTAGGGCCCAGCAGCGCAAGCGGGACGGGCCCGACCTTGCCGAG | |
| TATTACTATGATGCACACCTATGACCCAAGGCCCTCATAAAGATACCATG | |
| Human Neublasmin variant B genomic nucleotide sequence | |
| (variant of SEQ ID NO 1) | |
| gagactgctg gccctgcccc agggcaggtg ctgacgcagg cttggaatga | 45960493 |
| aggccctttg tgaggtggcc ctgggagccg gcaacgggtt ccgtcctgcc | 45960443 |
| AGCATAGGGA CGACAGGAGC CAGGACCCGT GTAGGAGATG GTTCTCGCCA | 45960393 |
| TGCTGGGGGC TCTGCACCCC CGGGCTGGGC TCAGCCTCTT CCTCCACCTC | 45960343 |
| ATCCTGGCAG TGGCACTGCT TCGCTCCCAG CCTCTGAGgt gactcctggg | 45960293 |
| gcacagaggg cagggcactt gggccggatg gtggaggtgc tgcccctccc | 45960243 |
| ctctctggag tggggctggc cctggtaccc tgggcgttcg agggcttgcc | 45960193 |
| aagtgccccc acactgagcc atccccccat tcacaaggag gtggcggtgg | 45960143 |
| ccttctgggc cagggctgag caccccgaca tctcccacag GTCTCAGCGG | 45960093 |
| TCTGTTCCTG AGGCATTTTC CGCCCCCCTG GAACTCTCGC AGCCACTTTC | 45960043 |
| CGGCCTGGTG GATGgtagta tctctcttgt ccccttcccc tacccatccc | 45959993 |
| tgacagtgga gacgccccta agaggtgccc caagtccagc cttcaggggc | 45959943 |
| ttgtcatccc aaaccacatc tgttcaaggg tgacttcagt tccatcagag | 45959893 |
| ccagctgccc cctaccaccc atgccctctt ttctctcttt agACTATGGC | 45959843 |
| ATCCTCCCCA AGCACCCAAG GCCGCGAGGG CCTCGACCCC TCCTGTCTAG | 45959793 |
| GGCCCAGCAC CGCAAGCGGG ACGGGCCCGA CCTTGCCGAG TATTACTATG | 45959743 |
| ATGCACACCT ATGACCCAAG GCCCTCATAA AGATACCATG tgtgacaaag | 45959693 |
| gcttggcttt cctgggctgg gagcgagacg gggacgggag gggcccatgc | 45959643 |
| agatacatcc actgcagtcc agacaggaaa tggcacttta | |

Human Neublasmin variant B cDNA (SNP G→C at pos.280)

(SEQ ID NO 5)
AGCATAGGGACGACAGGAGCCAGGACCCGTGTAGGAGATGGTTCTCGCCATGCTGGGGGCTC
TGCACCCCCGGGCTGGGCTCAGCCTCTTCCTCCACCTCATCCTGGCAGTGGCACTGCTTCGC
TCCCAGCCTCTGAGGTCTCAGCGGTCTGTTCCTGAGGCATTTTCCGCCCCCCTGGAACTCTC
GCAGCCACTTTCCGGCCTGGTGGATGACTATGGCATCCTCCCCAAGCACCCAAGGCCGCGAG
GGCCTCGACCCCTCCTGTCTAGGGCCCAGCACCGCAAGCGGGACGGGCCCGACCTTGCCGAG
TATTACTATGATGCACACCTATGACCCAAGGCCCTCATAAAGATACCATG

Protein Variants:

Human Neublasmin variant 1 (98 amino acids; SEQ ID NO 2, 4)

MVLAMLGALH PRAGLSLFLH LILAVALLRS QPLRSQRSVP
EAFSAPLELS QPLSGLVDDY GILPKHPRPR GPRPLLSRAQ
QRKRDGPDLA EYYYDAHL

Human Neublasmin variant 2 (94 amino acids) (N-4 version) (variant of SEQ ID NO 5)

MLGALH PRAGLSLFLH LILAVALLRS QPLRSQRSVP EAFSAPLELS
QPLSGLVDDY GTLPKHPRPR GPRPLLSRAQ QRKRDGPDLA
EYYYDAHL

Human Neublasmin variant A mature (68 amino acids) (Minus 30aa signal peptide) (SEQ ID NO 9)

QPLRSQRSVP EAFSAPLELS QPLSGLVDDY GILPKHPRPR
GPRPLLSRAQ QRKRDGPDLA EYYYDAHL

Human Neublasmin variant B (98 amino acids) (SNP G→C changes aa Q→H at pos.81) (SEQ ID NO 7)

MVLAMLGALH PRAGLSLFLH LILAVALLRS QPLRSQRSVP
EAFSAPLELS QPLSGLVDDY GILPKHPRPR GPRPLLSRAQ
HRKRDGPDLA EYYYDAHL

Human Neublasmin variant B (94 amino acids) (N-4 version) (SNP G→C changes aa Q→H at pos.81) (SEQ ID NO 8)

MLGALH PRAGLSLFLH LILAVALLRS QPLRSQRSVP EAFSAPLELS
QPLSGLVDDY GILPKHPRPR GPRPLLSRAQ HRKRDGPDLA
EYYYDAHL

Human Neublasmin variant B mature (68 amino acids) (SNP G→C changes aa Q→H at pos.81) (SEQ ID NO 10)

QPLRSQRSVP EAFSAPLELS QPLSGLVDDY GILPKHPRPR
GPRPLLSRAQ HRKRDGPDLA EYYYDAHL

Human Neublasmin N-terminal peptide (38 amino acids) (SEQ ID No 26)

QPLRSQRSVP EAFSAPLELS QPLSGLVDDY GILPKHPR

Human Neublasmin N-terminal peptide (40 amino acids) (SEQ ID No. 27)

RSQPLRSQRS VPEAFSAPLE LSQPLSGLVD DYGILPKHPR

Human Neublasmin C-terminal peptide, variant A (30 amino acids) (SEQ ID No. 28)

PRGPRPLLSR AQQRKRDGPD LAEYYYDAHL

Human Neublasmin C-terminal peptide, variant B (30 amino acids) (SEQ ID No. 29)

PRGPRPLLSR AQHRKRDGPD LAEYYYDAHL

Mouse Neublasmin N-terminal peptide (40 amino acids) (SEQ ID No. 30)

RPQPQRSQQS VPEEFSAPLE LLQPLSGLVD DYGLRPKHPR

Mouse Neublasmin C-terminal peptide (30 amino acids) (SEQ ID No. 31)

PGGPRPLLSQ AQQRKRDGPN MADYYYDVNL

Rat Neublasmin N-terminal peptide (40 amino acids) (SEQ ID No. 32)

RPQPQRSQRS VPEEFSAPLE LLQPLSGLVD DYGLRPKHPR

Rat Neublasmin C-terminal peptide (30 amino acids) (SEQ ID No. 33)

PGGPRPLLSQ AQQRKRDGPD MADYYYDVNP A

Chimpanzee Neublasmin variant 1 (99 amino acids) (SEQ ID No 23)

MVLAMLGALH PRAGLSLFLH LILAVALLRS QPLRRSQRSV
PEAFSAPLEL SQPLSGLVDD YGILPKHPRP QGPRPLLSRA
QQRKRDGPDL AEYYYDAHL

Chimpanzee Neublasmin variant 2, N-4 (95 amino acids) (SEQ ID No. 24)

MLGALHPRAG LSLFLHLILA VALLRSQPLR RSQRSVPEAF
SAPLELSQPL SGLVDDYGIL PKHPRPQGPR PLLSRAQQRK
RDGPDLAEYY YDAHL

Chimpanzee Neublasmin mature protein (SEQ ID No. 25)

QPLRRSQRSV PEAFSAPLEL SQPLSGLVDD YGILPKHPRP
QGPRPLLSRA QQRKRDGPDL AEYYYDAHL

Chimpanzee Neublasmin N-terminal peptide (SEQ ID NO 34)

QPLRRSQRSV PEAFSAPLEL SQPLSGLVDD YGILPKHPR

Chimpanzee Neublasmin C-terminal peptide (SEQ ID NO 35)

PQGPRPLLSR AQQRKRDGPD LAEYYYDAHL

Sequence Listing:
1. Human Neublasmin genomic nucleotide sequence
2. Human Neublasmin encoded full length protein (XP_{—}096472)
3. Human Neublasmin valiant A cDNA (XM_{—}096472)
4. Human Neublasmin variant A full length protein
5. Human Neublasmin variant A, N-4.
6. Human Neublasmin variant B cDNA
7. Human Neublasmin variant B full length protein
8. Human Neublasmin variant B, N-4
9. Human Neublasmin variant A mature protein
10. Human Neublasmin variant B mature protein
11. Mouse Neublasmin cDNA (NM_{—}183112)
12. Mouse Neublasmin full length protein
13. Mouse Neublasmin N-4
14. Mouse Neublasmin mature protein
15. Rat Neublasmin cDNA (ENSRNOG00000006884)
16. Rat Neublasmin full length protein
17. Rat Neublasmin, N-4
18. Rat Neublasmin mature protein
19. Human Neublasmin, DNA coding for variant A mature protein
20. Human Neublasmin, DNA coding for variant B mature protein
21. Mouse Neublasmin, DNA coding for mature protein
22. Rat Neublasmin, DNA coding for mature protein
23. Chimpanzee full length protein
24. Chimpanzee, N-4 protein
25. Chimpanzee mature protein
26. Human N-terminal peptide (38 amino acids)
27. Human N-terminal peptide (40 amino acids)
28. Human C-terminal peptide, variant A (30 amino acids)
29. Human C-terminal peptide, variant B (30 amino acids)
30. Mouse N-terminal peptide (40 amino acids)
31. Mouse C-terminal peptide (30 amino acids)
32. Rat N-terminal peptide (40 amino acids)
33. Rat C-terminal peptide (30 amino acids)
34. Chimpanzee, N-terminal peptide
35. Chimpanzee, C-terminal peptide
36. Human Neublasmin antigen, PEP1 (14 amino acids)
37. Human Neublasmin antigen, PEP2 (15 amino acids)
38. Human Neublasmin antigen, PEP3 (14 amino acids)
39. Chimpanzee, Pan troglodytes, cDNA
40. Chimpanzee protein (identical to SEQ ID NO 23)
41. hNeublasmin-V5 fusion protein.

### Example 3

### Bioinformatics

Variants:

Variant 1 (human): 98 amino acids

Variant 2 (human): 94 amino acids, lacking the 4 N-terminal residues of variant 1. Uses alternative start-codon.

Correspondingly, in mouse and chimpanzee, a 95 amino acid variant exists (lacks 4 N-terminal residues of 99 aa variant). In rat, a 96 amino acid variant exists (lacks 4 N-terminal residues of 100 aa variant).

Evidence for Variants:

S-curve from SignalP prediction is lowered for the first 4 residues

Start codon prediction (NetStart) predicts similar probabilities for the two potential ATG start codons

GenScan gene prediction on human genomic fragment (882 bp) predicts CDS start at ATG-codon corresponding to variant 2

Protein Processing:

Secreted Protein:

SignalP-NN and -HMM methods predict cleavage site at positions shown in Example 4. Based on signal peptide prediction from SignalP-NN and SignalP-HMM. Signal peptide predicted at homologous position in Mouse, Rat and Chimpanzee Neublasmin ProtFun-SecretomeP predicts ‘secretion’ category with high score.

Proprotein Processing:

Although furin- and general-type proprotein cleavage is predicted in human Neublasmin by the ProP method, this site is not conserved in mouse Neublasmin and probably not physiologically relevant

Expression Data:

Human ESTs: pooled germ cell tumors; testis

Inventor's Affymetrix U133: Expression in human Ventral Mesencephalon and Dorsal Mesencephalon midbrain

Mouse ESTs: testis; medulla oblongata; skin; pituitary gland; pooled lung tumors; whole skin; Newborn Brain

Protein Function: (ProtFun Prediction)

| | |
|---|---|
| Probability | Odds |

Human Neublasmin, variant A (SEQ ID No 4)

| | | |
|---|---|---|
| Hormone | =>0.202 | 31.030 |
| Growth_factor | 0.040 | 2.870 |

Human Neublasmin, variant B (SEQ ID No 7)

| | | |
|---|---|---|
| Hormone | =>0.205 | 31.537 |
| Growth_factor | 0.040 | 2.871 |

Mouse Neublasmin (SEQ ID No 12)

| | | |
|---|---|---|
| Hormone | =>0.271 | 41.628 |
| Growth_factor | 0.023 | 1.652 |

Rat Neublasmin (SEQ ID No 16)

| | | |
|---|---|---|
| Hormone | =>0.254 | 39.095 |
| Growth_factor | 0.060 | 4.315 |

Chimpanzee Neublasmin (SEQ ID No 23)

| | | |
|---|---|---|
| Hormone | =>0.210 | 32.257 |
| Growth_factor | 0.029 | 2.051 |

Hit Against Most Relevant Genes.

Does not seem to have homology to any other gene (known or unknown)

PTM Analysis:

Glycosylation

NetOGlyc: no sites in human Neublasmin

NetNGlyc: No ASN(N)-residues in sequence.

PFAM: There were no matches to Pfam-A (including borderline matches) for UNKNOWN-QUERY There were no matches to Pfam-B for UNKNOWN-QUERY

InterPro: no relevant matches

Conclusion: No obvious modifications predicted. No hits to other domains.

Further truncations are not likely.

### Example 4

### Prediction of Signal Sequences

SignalP predictions for human full length Neublasmin (SEQ ID Nos. 2 and 4).

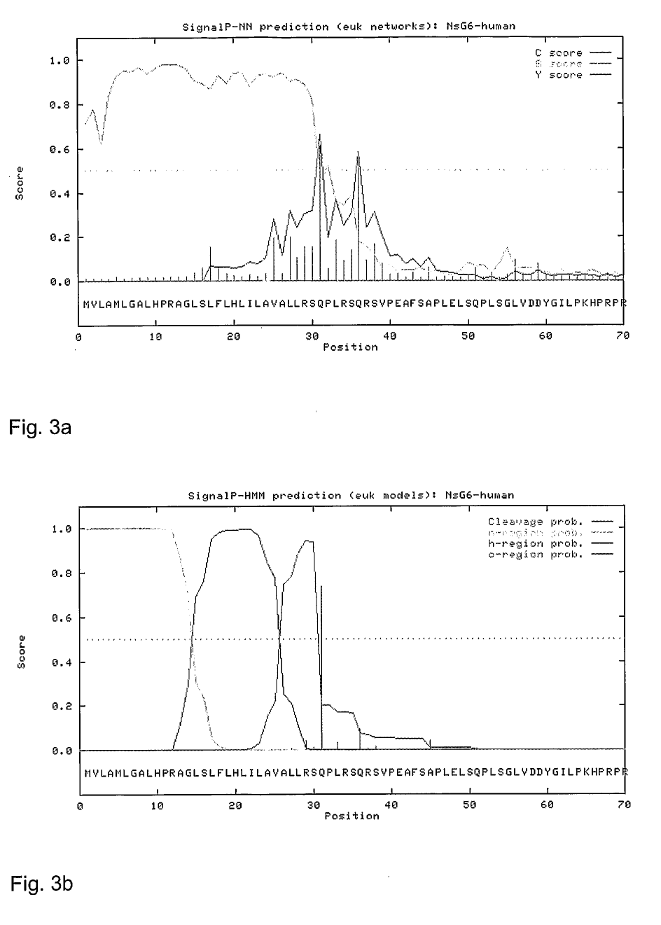

SignalP-NN (see FIG. 3a)

>Neublasmin-human length = 70

| # Measure | Position | Value | Cutoff | signal peptide? |
|---|---|---|---|---|
| max. C | 31 | 0.615 | 0.33 | YES |
| max. Y | 31 | 0.663 | 0.32 | YES |
| max. S | 12 | 0.979 | 0.82 | YES |
| mean S | 1-30 | 0.901 | 0.47 | YES |

# Most likely cleavage site between pos. 30 and 31: LRS-QP

SignalP-HDM (see FIG. 3b)

>Neublasmin-human

Prediction: Signal peptide

Signal peptide probability: 0.998

Signal anchor probability: 0.002

Max cleavage site probability: 0.739 between pos. 30 and 31

SignalP predictions for full length mouse Neublasmin (SEQ ID No 12)

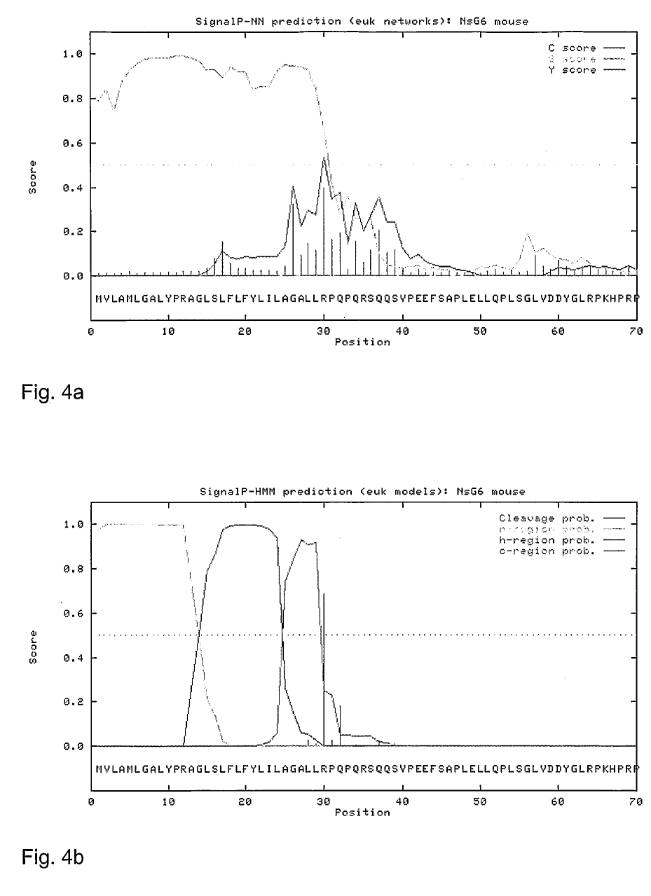

SignalP-NN (see FIG. 4a)

>Neublasmin_mouse length = 70

| # Measure | Position | Value | Cutoff | signal peptide? |
|---|---|---|---|---|
| max. C | 30 | 0.394 | 0.33 | YES |
| max. Y | 30 | 0.532 | 0.32 | YES |
| max. S | 11 | 0.993 | 0.82 | YES |
| mean S | 1-29 | 0.918 | 0.47 | YES |

# Most likely cleavage site between pos. 29 and 30: ALL-RP

SignalP-HDM result (see FIG. 4b)

>Neublasmin_mouse

Prediction: Signal peptide

Signal peptide probability: 0.981

Signal anchor probability: 0.018

Max cleavage site probability: 0.683 between pos. 29 and 30

SignalP predictions for full length rat Neublasmin (SEQ ID No 16)

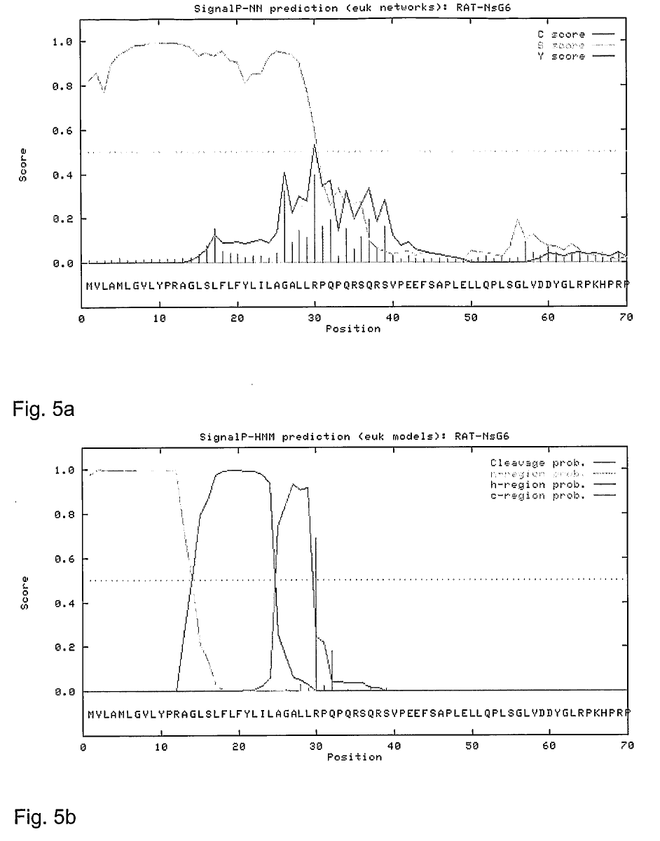

SignalP-NN result (see FIG. 5a)

>RAT-Neublasmin length = 70

| # Measure | Position | Value | Cutoff | signal peptide? |
|---|---|---|---|---|
| max. C | 30 | 0.394 | 0.33 | YES |
| max. Y | 30 | 0.532 | 0.32 | YES |
| max. S | 11 | 0.995 | 0.82 | YES |
| mean S | 1-29 | 0.922 | 0.47 | YES |

# Most likely cleavage site between pos. 29 and 30: ALL-RP

SignalP-HMM result (see FIG. 5b)

>RAT-Neublasmin

Prediction: Signal peptide

Signal peptide probability: 0.978

Signal anchor probability: 0.022

Max cleavage site probability: 0.687 between pos. 29 and 30

SignalP predictions for Human Neublasmin-N-4 variant (SEQ ID No 5):

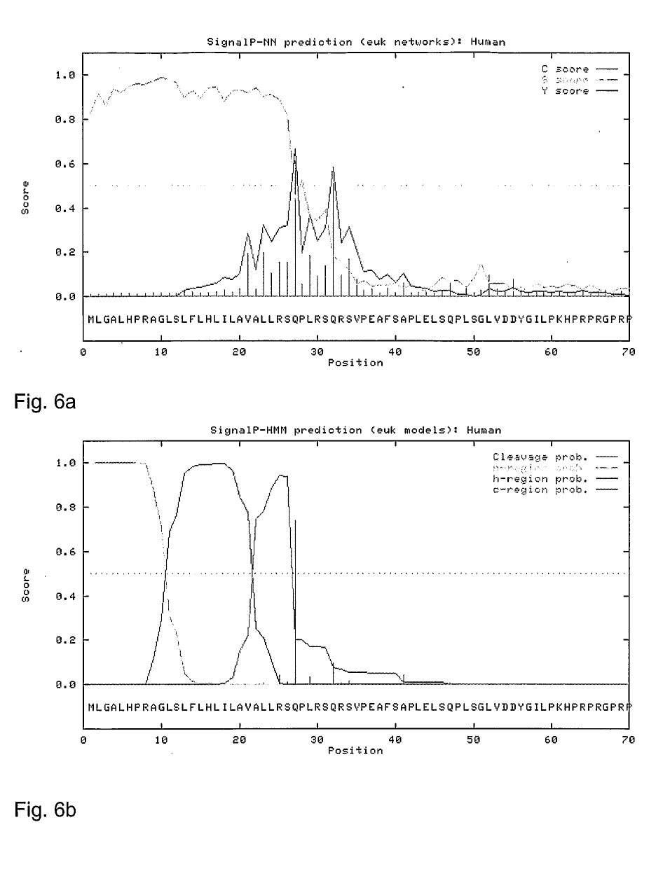

SignalP-NN result (see FIG. 6a)

>Human Neublasmin, N-4 variant length = 70

| # Measure | Position | Value | Cutoff | signal peptide? |
|---|---|---|---|---|
| max. C | 27 | 0.615 | 0.33 | YES |
| max. Y | 27 | 0.666 | 0.32 | YES |
| max. S | 10 | 0.989 | 0.82 | YES |
| mean S | 1-26 | 0.923 | 0.47 | YES |

# Most likely cleavage site between pos. 26 and 27: LRS-QP

SignalP-HMM result (see FIG. 6b)

>Human Neublasmin N-4 variant

Prediction: Signal peptide

Signal peptide probability: 0.996

Signal anchor probability: 0.004

Max cleavage site probability: 0.738 between pos. 26 and 27

### Example 5

### Detection of Neublasmin mRNA Expression in Testis by RT-PCR Analysis

Purified total RNA from human testis purchased from Clontech was used for the analysis following treatment with Dnase to remove traces of chromosomal DNA. The absence of genomic DNA was verified by amplification with primers specific for chromosomal markers on chromosomes 7, 13 and 20.

Dnase treated RNA was reverse transcribed to cDNA using MGCT₁₁V primer (V=A, G or C) using the manufacturers protocol.

PCR primers 891 and 892 were used for the analysis. These primers amplify 210 bp spanning the intron between exon 2 and 3. The PCR reactions were carried out in a 15-μl volume containing 0.5 unit of Taq polymerase (Amersham Pharmacia), 50 mM Tris-HCl, pH 7.5, 50 mM KCl, 1.5 mM MgCl₂, 10 pmol of primers 891 (GGTCTGTTCCTGAGGCATTTTCC) and 892 (GTATCTTTATGAGGGCCTTGGGTCA), 200 μM of each of the dNTPs, and RT product equivalent to 23 ng total RNA. Reaction including dH₂O instead of cDNA was included as control. After a pre-denaturation step at 96° C. for 2 min, the PCR reactions were run as 35 cycles of a denaturation step at 96° C. for 20 s, annealing step at 56° C. for 20 s and an elongation step at 72° C. for 20 s. PCR products were separated on 2% agarose gels and visualized by ethidium bromide.

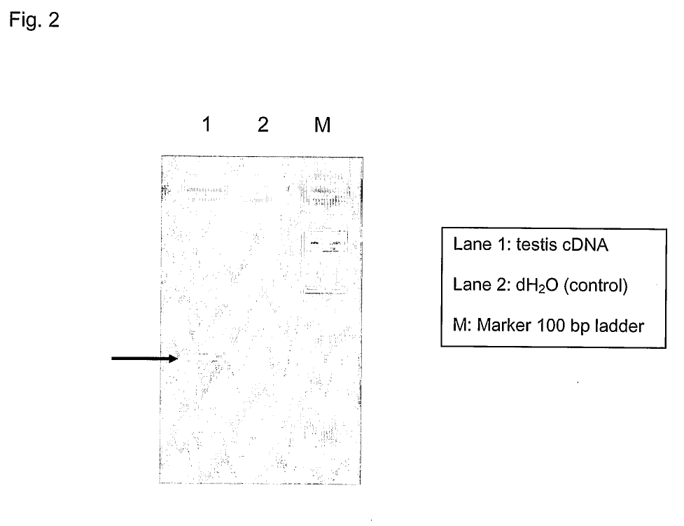

As seen in FIG. 2, a band of the expected size (210 bp) is amplified from testis cDNA templates.

In summary, this result indicates that the biological functions of Neublasmin may include testis related disorders, such as but not limited to: spermatogenesis, male sterility, impotence, erectile dysfunction, cancer, and germ cell tumours.

### Example 6

### Gene-Chip Experiments

The human material comes from discarded tissue pieces obtained from electively terminated pregnancies using the regular vacuum aspiration technique. The collection of residual tissue for the study is approved by the Human Ethics Committee of the Huddinge University Hospital, Karolinska Institute (Diary Nr. 259/00) and Lund University (970401), and is in accordance with the guidelines of the Swedish National Board of Health and Welfare (Socialstyrelsen), including an informed consent from the pregnant women seeking abortions. Recovered nervous tissue is micro-dissected within 2 hours of surgery and appropriate tissue fragments are further dissociated for cell isolation.

RNA Isolation:

Human fetal tissue (8 weeks) was obtained in two rounds, both 8-weeks gestation age. Dissected VM and DM regions were used for total RNA isolation with good results and yields.

Total RNA was isolated with the Trizol extraction following the manufacturer's instruction (Invitrogen) from ventral and dorsal mesencephalic regions subdissected from human fetal tissue, 8 weeks gestational age. To concentrate RNA and to remove traces of chromosomal DNA, Rneasy columns combined with the RNase-Free DNase Set are used following the manufacturer's instructions.

From 5 μg of total RNA, biotinylated cRNA is prepared and fragmented as described in Affymetrix protocols (GeneChip Expression Analysis, Technical Manual 2000) and hybridized (15 μg) to Affymetrix Human U133B GeneChips (containing approximately 22,000 genes) according to manufacturer's instructions. Scanned images are analyzed and converted to expression index values using the GenePublisher analysis software package (Knudsen S, Workman C, Sicheritz-Ponten T, Friis C. (2003) “GenePublisher: Automated analysis of DNA microarray data.”, Nucleic Acids Res. 31(13):3471-6.). Expression values in the range of 135-153 are observed in the samples from dorsal- and ventral midbrain, indicating a level significantly above the background level (approximately 50).

Based on the GeneChip expression values we conclude that Neublasmin is transcriptionally active in the human midbrain.

### Example 7

### Testing in PC12 Cells

Generation of Virus Stock:

Neublasmin coding sequence (SEQ ID No 3 or 6) is subcloned into pLCXSn using appropriate restriction sites. To generate virus stocks, the resulting retroviral transfer vector is cotransfected into 293T cells with two helper plasmids (pVPack and pVPack-VSV-G) providing the necessary viral genes, gag-pol and env, respectively, in trans. Briefly, 2×10⁶ 293T cells are seeded in each of 6 T75 culture flasks. The next day, each T75 flask is transfected with 6 μg pVPack-GP, 5 μg pVPack-VSV-G and 7 μg of transfer vector using Lipofectamine+following the manufacturer's instructions (Invitrogen). Virus-containing medium is harvested 2-3 days after the transfection and filter-sterilized through a 0.45 μm cellulose acetate or polysulphonic filter. The virus is pelleted by ultracentrifugation at 50,000×g for 90 minutes at 4° C. and then resuspended in DMEM medium. Virus is titrated using a reverse transcriptase (RT) assay (Current Protocols). The number of transducing units (TU)/ml is calculated from the resulting RT activity and frequency of fluorescent cells obtained by transduction of NIH3T3 cells with an equivalent GFP retrovirus. The virus stock is stored in aliquots at −80° C. until use.

Transduction of PC12 Cells:

PC12 cells are cultured in Dulbecco's modified Eagle's medium (DMEM) with 4.5 g/l glucose and glutamax (Life Technologies #32430-027) with 7.5% donor horse serum (Life Technologies #16050-098) and 7.5% FBS (Life Technologies # 10099-141) in the presence of 5% CO₂ at 37° C. Medium is changed every 2-3 days and cells are subcultured 1:3-1:6 twice a week by tapping the flask and dispensing into new flasks. The day before transduction, cells are seeded in 6-well plates coated with collagen. Virus is added from the stock solution to 1 ml cell culture medium together with or without 5 μg/ml (final conc.) polybrene. The virus is incubated with the cells for at least 3 hours in a CO₂ incubator. GFP retrovirus is added to a parallel culture to estimate transduction efficiency and to serve as control.

Effect on PC12 Differentiation:

Cultures in 6-well plates are followed and scored for the number of neurite bearing cells after 2-5 days.

Effect on PC12 Survival:

Transduced cells from 6-well plates are reseeded in 96-well plates coated with collagen in culture medium. The following day, medium is changed to serum-free DMEM and cell viability is measured after 24-72 hr using the MTS assay following the manufacturer's instructions (Promega).

The assay may also be carried out with conditioned medium harvested from mammalian cells expressing Neublasmin.

A positive effect in either the neurite outgrowth and/or the survival assay is indicative of a therapeutic effect of the encoded protein for CNS disorders.

### Example 8

### Neublasmin Quantitative Expression Data

Method

Primers amplifying cDNA fragments of 100-350 bp were designed using CloneManager software.

Total RNAs derived from fetal and adult human tissues were purchased from Clontech, Dnase treated to remove residual chromosomal DNA and used as templates for cDNA synthesis using an RnaseH deficient reverse transcriptase. cDNA equivalent to 21 ng total RNA was used for each PCR reaction which were carried out using a Opticon 2 light cycler from MJ research.

Real-time PCR was performed in an Opticon 2 thermocycler (MJ Research), using LightCycler-FastStart DNA Master SYBR Green I kit (Roche). Studies were carried out in duplicates using primers 5′ oligo: 5′-CCGACCTTGCCGAGTATTACTATGA-3′, annealing at bp 45884709-45884733 of the May 2004 assembly at the UCSC genome browser and 3′ oligo: 5′-CTGTCTGGACTGCAGTGGATGTATC-3′, annealing at bp 45884584-45884608 of the may 2004 assembly at the UCSC genome browser amplifying a 150 bp fragment of the Neublasmin cDNA located in the Neublasmin ORF region. For Real-Time PCR, a standard curve was prepared by serial dilution of a gel-purified PCR product, prepared using the above primers. The standard curve was used to verify that crossing-point values (C(T)) of all samples were within the exponential range of the PCR reaction and to calculate final expression levels. All RT-PCR amplifications were performed in a total volume of 10 μl containing 2 mM MgCl₂, 12% sucrose and 1× reaction buffer included in the LightCycler kit. PCR cycling profile consisted of 95° C., 10′>>35 cycles: 95° C., 10″>>60° C., 20″>>72° C., 20″>>plate read 72° C., 2″. The specificity of the amplification reaction was determined by performing a melting curve analysis of the PCR fragments by slowly raising the temperature from 55° C. to 95° C. with continuous data acquisition.

For normalization purposes, all cDNAs were subjected to real-time PCR using primers for β₂-microglobulin (B2M, 5′-TGTGCTCGCGCTACTCTCTC-3′ and 5′-CTGAATGCTCCACTTTTTCAATTCT-3′). Standard curves for β₂-microglobulin were prepared similar to Neublasmin. β₂-microglobulin gene real-time PCR was done using the same kit as for the target gene, except a different annealing temperature was used.

β₂-microglobulin expression levels were determined from the standard curve and the relative expression levels were used to normalize expression levels of the target genes in the tissues that were analyzed. Following normalization, relative expression levels of the target gene were calculated using the tissue with the lowest expression as a reference (spleen). Normalized data in FIG. 7 should be interpreted with caution as β₂-microglobulin levels vary between some tissues.

Data are shown in FIG. 7. The corresponding C(T) values are shown in the table below.

| Tissue | C(T) |
|---|---|
| adrenal gland | 30.0207 |
| bone morrow | 29.0337 |
| brain, cerebellum | 28.0217 |
| brain(whole) | 27.269 |
| heart | 31.0843 |
| kidney | 28.6103 |
| liver | 32.216 |
| lung | 31.026 |
| placenta | 29.6133 |
| prostate | 29.4043 |
| salivary gland | 29.4723 |
| skeletal muscle | 30.3693 |
| spleen | 29.9217 |
| testis | 22.6023 |
| thymus | 28.6237 |
| thyroid glaqnd | 28.7513 |
| trachea | 29.524 |
| uterus | 29.9443 |
| colon w/mucosal lining | 30.3853 |
| small intestine | 29.9203 |
| Spinal cord | 28.0633 |
| Fetal liver | 31.0037 |
| Fetal brain | 27.5897 |
| Pancreas | 31.435 |
| Caudate nucleus | 28.91 |
| Amygdala | 28.9337 |
| Corpus callosum | 28.6047 |
| Hippocampus | 28.519 |
| Thalamus | 29.0627 |
| Pituitary | 28.1207 |

The highest level of Neublasmin expression is detected in testis. Lower but still detectable levels are also seen in fetal and adult brain in addition to adult cerebellum. This expression analyses indicates that Neublasmin is of diagnostic value and a potential therapeutic candidate for the treatment of testicular disorders. Furthermore, Neublasmin may also be capable of exerting effects in the brain in particular the cerebellum.

### Example 9

### Expression Analysis

Primers (Human & Mouse):

| | |
|---|---|
| ns-gene-1 | CTCGCCATGCTGGGGGCTCT |
| ns-gene-2T3 | AATTAACCCTCACTAAAGGGCTGGGCTCA |
| ns-gene-3T7 | TAATACGACTCACTATAGGGCGCTGCTGGGC |
| ns-gene-4 | CCATCCCGCTTGCGCTGCT |

All primers are compatible with mouse and human Neublasmin cDNA sequences, see alignment with primer sequences underlined below

ns-mouse-gene.seq×ns-gene.seq . . .

Percent Similarity: 80.675 Percent Identity: 80.675

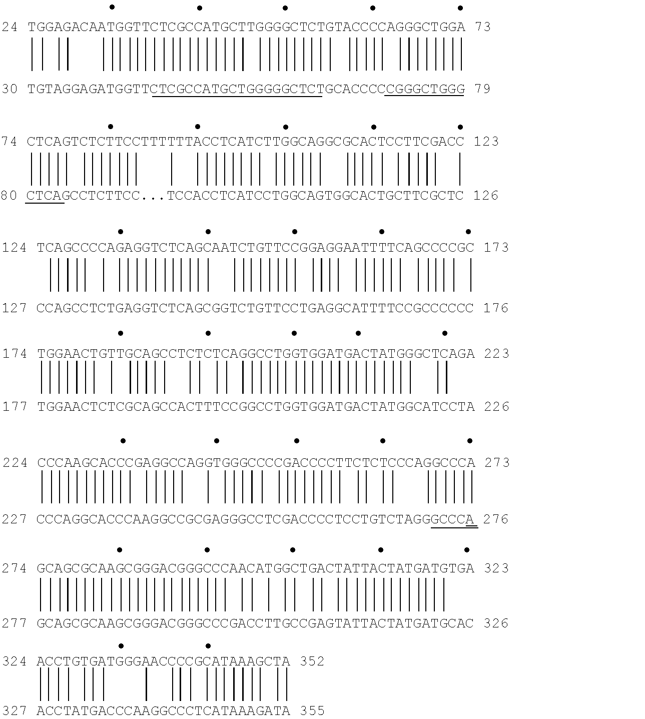

RT-PCR Analysis

Neublasmin expression in was analyzed during mouse spermatogenesis. Testes were sampled from postnatal development as indicated in FIG. 8A. RNA was purified and cDNA prepared. mNeublasmin expression was analyzed by RT-PCR (40 cycles) using primers [ns-gene-1 and ns-gene-4] amplifying a fragment of 254 bp from mouse Neublasmin cDNA. Expression analyzed using the same primers and RT-PCR (40 cycles) in normal human testis and in CIS, (carcinoma in situ) is also shown (the expected product amplified from human cDNA is 251 bp). As seen in FIG. 8A a product of the correct size is amplified from mouse testis cDNA sampled at pn 22-52 whereas only larger (unspecific) products are amplified from younger ages. Also a specific band of the expected size could be amplified from both cDNAs derived human material included in the analysis. The amplified band has been sequenced and its identity as a Neublasmin band has been confirmed.

In FIG. 8B, RT-PCR analysis (40 cycles) was carried out as described for the analysis shown in FIG. 8A except that cDNAs (all of human origin) derived from a number of different testicular tumours (Seminoma, teratoma and EC=Embryonal Carcinoma) and two cell lines derived (NT2 and 2102EP) were included in the analysis. Samples derived from NT2 and 2102EP cells treated with retinoic acid (=RA) for 7-15 days (d) were also included in addition to samples derived from the undifferentiated (u-diff) cultures. NT2 and 2102EP are cloned cell lines both derived from EC. NT2 cells are capable of differentiating after exposure to RA whereas 2102Ep cells are not (Mavilio et al., 1988. Differentiation. 37:73-9). As seen in FIG. 8B, Neublasmin is expressed in all tissues and cells tested except one sample derived from an EC. The amplified band has been sequenced and its identity as a Neublasmin band has been confirmed.

### Example 9

### In Situ Hybridisation

In Situ Hybridization (ISH)

The generation of probes and the ISH were carried out as described previously (Hoei-Hansen et al., 2004 Mol. Hum. Reprod. 10, 423-431). Briefly to generate probes for ISH were prepared by amplification of a fragment using human Neublasmin testis cDNA as template and primers where an added T3-promotor sequence was added to the 5′ primer (ns-gene-2T3) and a T7 promotor sequence (ns-gene-3T7) was added to the 3′ primer. The anti-sense probe (transcribed using T7 polymerase) and the sense probe (transcribed using T3 polymerase) were hybridized to sections of a biopsy with human testicular biopsy containing CIS (carcinoma in situ).

In order to investigate which cell types that express Neublasmin, we performed non-radioactive ISH on a testicular biopsy containing CIS. CIS cells contain large glycogen-rich vacuoles that disappear during fixation, thus the remnants of cytoplasm are usually visible as a thin ring attached to the cell membrane. As seen in FIG. 9, no signal or a very low signal was detected using the sense-probe. In contrast, expression in both CIS cells and in undifferentiated germinal cells was clearly detected with the anti-sense probe. However, the signal detected from the CIS cells was much stronger than from normal germ cells. Due to differences in cellularity, these results are in agreement with the RT-PCR data. There are considerable more spermatocytes/spermatogonia in normal testes than CIS cells in testes with CIS, The staining in CIS cells appears to be both cytoplasmic and nuclear.

### Example 10

### Real-Time PCR on Neublasmin Mouse Orthologue

Materials & Methods:

Primers:

The following primers were used for real-time PCR:

| | |
|---|---|
| GAPDH: | |
| mGAPDH-s904: | 5′-AACAGCAACTCCCACTCTTC-3′ |
| mGAPDH-as1067: | 5′-TGGTCCAGGGTTTCTTACTC-3′ |
| mALDH1A1: | |
| mALDH1A_59Ofwd, | 5′-AAACTCCTCTCACGGCTCTTC-3′ |
| mALDH1A1_859rev, | 5′-CAATGTCCAAGTCGGCATCTG-3′ |
| mOTX2: | |
| mOTX2_269fwd, | 5′-CCGCCTTACGCAGTCAATGG-3′ |
| mOTX2_611rev, | 5′-TCACTTCCCGAGCTGGAGAG-3′ |
| 1491, mNsG6s(65): | AGGGCTGGACTCAGTCTCTTC |
| 1492, mNsG6as(340): | GGGTTCCCATCACAGGTTCAC |

Preparation of Expression Panel:

Tissue from different brain regions of E10.5, E11.5, E13.5, P1 and adult mice was isolated and RNA prepared by Trizol extraction. Subsequent on-column DNAse treatment using RNeasy spin columns was done to remove traces of gDNA and to further clean the RNA. Aliquots of 2.5 μg RNA was used as template for cDNA synthesis with an RNAseH deficient reverse transcriptase derived from MoMLV (SuperScript) and poly-dT pimer. cDNA from all samples were synthesised at the same time using the same mastermix to avoid variations. The final volume of the cDNA reaction was 120 μl, which was stored in aliquots at −80° C. to avoid repeated thawing and freezing. The expression panel consists of cDNA prepared from the following tissues; dorsal forebrain (DFB), ventral forebrain (VFB), ventral mesencephalon (VM), dorsal mesencephalon (DM) and spinal cord (SC) from 10.5 and 11.5 weeks old embryos. In addition, cortex (CTX), medial and lateral ganglionic eminences (MGE/LGE), DM, VM and SC from 13.5 weeks old embryos were included. Furthermore, from newborn mouse (P1), cerebellum (Cb), CTX, VM, DM, and MGE/LGE were used and finally Cb, CTX, VM, DM, and SC were used from adult mouse.

Real Time PCR Expression Analysis:

For real-time PCR expression analysis, approximately 20 ng of each cDNA was used as template. Real-time PCR was performed in an Opticon-2 thermocycler (MJ Research), using LightCycler-FastStart DNA Master SYBR Green I kit (Roche). Studies were carried out in duplicates using the primers described above. For real-time PCR, a standard curve was prepared by serial dilution of a gel-purified PCR product, prepared using the above primers. The standard curve was used to verify that crossing-point values (CT) of all samples were within the exponential range of the PCR reaction and to calculate final expression levels. All real-time PCR amplifications were performed in a total volume of 10 μl containing 3 mM MgCl₂, 12% sucrose and 1× reaction buffer included in the LightCycler kit. PCR cycling profile consisted of a 10 minutes pre-denaturation step at 98° C. and 35 three-step cycles at 98° C. for 10 seconds, at 62° C. (mGAPDH), 65° C. (mALDH1A1), 65° C. (mOTX2), 63° C. (mNeublasmin) for 20 seconds and at 72° C. for 20 seconds. Following the extension step of each cycle, a plate reading step was added (80° C., 2 seconds) to quantify the newly formed PCR products. The specificity of the amplification reaction was determined by performing a melting curve analysis of the PCR fragments by slowly raising the temperature from 52° C. to 95° C. with continuous data acquisition.

For normalization purposes, all cDNAs were subjected to real-time PCR using primers for the housekeeping gene GAPDH. Real-time PCR analysis of GAPDH was done as for the target genes. Housekeeping expression pattern was determined from the respective standard curves and the relative expression levels were used to normalize expression levels of the target genes in the tissues that were analysed. Following normalization with GAPDH, relative expression levels of the target genes were calculated using the tissue with the lowest expression as a reference.

Results: To verify tissue dissections and subsequent RNA isolation and cDNA preparation, the expression profile of the marker genes OTX2 and ALDH1A were investigated. OTX2 is expressed in the forebrain and primarily in the dorsal part of the midbrain with a posterior boundary at the isthmic organiser. The retinoid synthesizing enzyme ALDH1A1, is a specific marker of developing dopaminergic neurons in the ventral midbrain. Hence, the expression profile of these two genes can be used to validate the cDNA panel. It is apparent from FIG. 10, that the expression level of the housekeeping gene GAPDH differs less that 50% between tissues. In contrast, very differentiated expression profiles are observed for ALDH1A1 and OTX2. As expected, in the fetal tissues and at P1, ALDH1A1 is expressed almost exclusively in the ventral midbrain. Also as expected, during development, OTX2 is expressed in the forebrain and (dorsal) midbrain but not in the spinal cord. Together, this is evidence of a high quality expression panel of the developing mouse central nervous system (CNS).

The expression of Neublasmin is shown in FIG. 11. The C(t) values range from 24 to 27 in the brain sub-regions, thereby confirming the level of expression observed in human adult and foetal brain (FIG. 7). Expression increases from E10 through E11 to E13.5, from which time the expression levels remain at approximately the same level. Expression is relatively highest in Cerebellum (adult and P1) and dorsal mesencephalon (E13.5 and adult). This temporal and spatial distribution of expression coincides with the terminal differentiation of the central nervous system. This in turn indicates that Neublasmin may be involved in synapsis formation, neurotransmission, memory function and neuroplasticity.

### Example 11

### Detection of Recombinantly Produced Neublasmin Peptides

Cloning of Neublasmin-V5

To be able to detect heterologous produced Neublasmin protein, Neublasmin was PCR fused in frame to a V5 epitope tag. In addition, a six amino acid spacer region was inserted between Neublasmin and the V5 tag. The V5 tag is composed of 14 residues, GKPIPNPLLGLDST, derived from the P/V proteins of paramyxovirus SV5.

In a first PCR reaction, a 348 bp Neublasmin fragment was amplified from pHsC.hNeublasmin.W using primers 1611 and 1612. The forward primer has a BamHI site for later cloning, whereas the reverse primer has a 22 bp overlap with the spacer-V5 region. In a second PCR reaction, the 348 bp fragment was fused to the spacer-V5 region using primers 1611 and 1613. The 1613 primer is complementary to the spacer-V5 region and has a XhoI site for later cloning. In the same PCR reaction, the entire sequence encoding the fusion protein was amplified using primers 1611 and 1614, generating a 399 bp product. This fragment was digested with BamHI/XhoI and cloned into a similarly digested pHsCXW vector resulting in pHsC.hNeublasmin-V5.W. The plasmid construct was control digested with AlwNI, SacII and BamHI/XhoI and correct sequence of the Neublasmin-V5 region verified by complete sequencing of both strands. The verified DNA sequence of Neublasmin-V5 was translated and compared to the Neublasmin protein sequence as can be seen in FIG. 12.

Primer Sequences

#1611:
5′-CGACTCTAGAGGATCCAGGACCCGTGTAGGAGATGG-3′
#1612:
5′-TACCTTCGAACCGCGGGCCCTCTAGGTGTGCATCATAGT-3′
#1613:
5′-GATCCTCGAGTTACGTAGAATCGAGACCGAGGAGAGGGTTAGGGATA
GGCTTACCTTCGAACCGCGGGCCC-3′
#1614:
5′-GATCCTCGAGTTACGTAGAATCGAG-3′

Transfection of HEK293 Cells and Preparation of Samples for Western Blotting

HEK293T cells were transiently transfected with the expression construct containing Neublasmin-V5 (pHsC.hNeublasmin-V5.W). HEK293T cells were also mock transfected as a negative control. Briefly, HEK293T cells were seeded in 6-well plates (3×10̂5 cells/well) in DMEM supplemented with 10% FCS. After overnight incubation, cells were transfected with Lipofectamine+ using 2 μg DNA/well according to the manufacturer's instructions (Invitrogen). After 48 h, media were collected from the transfected cells and processed by centrifugation at 3000×g for 5 min. The cleared conditioned media were transferred to a clean eppendorftube and 5× Laemmli samplebuffer (10% SDS, 500 mM DTT, 300 mM Tris, pH 7.5, bromphenolblue) added. Cells were harvested for western blotting by adding 150 □l hot 1× samplebuffer per well.

Western Blotting

Samples were heated to 96° C. for 5 minutes and analysed by SDS PAGE on a 15% polyacrylamide gel using the MultiPhor II system according to the manufacturer's recommendations (Amersham Pharmacia, Denmark). The proteins were then blotted to PVDF membranes (BioRad, Denmark) that was immunostained using Anti V5-antibody (Invitrogen, R960-25, Part no. 46-0705) diluted 1:5000, followed by incubation with HRP-linked anti-mouse antibody (DAKO P0260) diluted 1:2000. Membranes were developed using the ECL system (Amersham Pharmacia, Denmark) and subjected to film exposure. Loading of MW low range: 2.512-16.949 (Pharmacia cat#80-1129-83) followed by GelCode Blue (24590, Pierce) staining of PVDF membranes was used for size determination.

Production of Polyclonal Neublasmin Antibodies

Three Neublasmin peptide sequences were selected for antibody production (PEP1: QPLRSQRSVPEAFS (SEQ ID NO 36) PEP2: QQRKRDGPDLAEYYY (SEQ ID NO 37) and PEP3: DGPDLAEYYYDAHL (SEQ ID NO 38)) based on their potential as antigens. Peptides were synthesised and antibodies produced by AnaSpec Inc (California, USA) according to the NIH 90 day standard protocol. Briefly, 4 mg of peptide conjugated to KLH was synthesised for antibody production. 2 rabbits were immunised with each conjugated peptide with standard immunization protocol, pre-immune serum, 4 injections, 3 bleeds with 25 ml serum/rabbit/each. Protocol: Subject: New Zealand White Rabbits; Adjuvant: Complete Freund's Adjuvant (CFA) initially, followed by incomplete Freund's; Adjuvant (IFA) for all subsequent injections; Immunogen: conjugated peptides. Amount of immunogen used for each immunization is 50 to 500 Ug.

Procedure: The immunogen is diluted to one ml with sterile saline and combined with one ml of the appropriate adjuvant. The antigen and adjuvant are mixed thoroughly to form a stable emulsion which is injected beneath the skin of the rabbit (subcutaneously) and provides enhanced immune response from the sustained presentation of the immunogen.

Bleeds: Blood is collected from the central ear artery with a 19 gauge needle and allowed to clot and retract at 37° C. overnight. The clotted blood is then refrigerated for 24 hours before the serum is decanted and clarified by centrifugation at 2500 rpm for 20 minutes

Schedule:

Time 0 Bleed 25 ml (yields 10 ml pre-immune serum). Immunize with antigen in CFA.

Week 3 Immunize with antigen in IFA

Week 6 Immunize with antigen in IFA

Week 7 Bleed 50 ml (yields 20 ml serum)

Week 10 Immunize with antigen in IFA

Week 11 Bleed 50 ml

Week 16 Bleed 50 ml

For each bleed, serum was isolated and the titre determined using peptide-specific ELISA. To select the most optimal bleed for antibody purification, serum was used for detection of the Neublasmin-V5 protein in western blotting (1:1000) using HRP-linked anti-rabbit Ab (Amersham NA 934) as secondary antibody. On the basis of this, PEP1 and PEP3 were selected for affinity purification.

Results

HEK293 cells were transfected with pHsC.hNeublasmin-V5.W and as a negative control, in parallel, cells were mock transfected. Cell lysates and conditioned medium were subsequently analyzed by anti V5 western blotting as seen in FIG. 13. One specific band of approximately 14 kDa was produced in the Neublasmin-V5 transfected cells, whereas there was no signal from the negative control. The size corresponds to the 13 kDa expected from Neublasmin-V5 protein with signal peptide included. In the conditioned medium, the negative control was blank and three specific bands were observed in the Neublasmin-V5 transfected cells. Again a 14 kDa band was seen in addition to an approximately 10 kDa and 5 kDa band. In size, 10 kDa corresponds to Neublasmin-V5 without signal peptide, whereas 5 kDa corresponds to the C-terminal fragment of Neublasmin-V5 after internal proteolytic cleavage. PC4 cleavage of Neublasmin-V5 protein will result in a 5.5 kDa C-terminal protein fragment. Similar results were obtained using CHO cells for expression.

The lysate and conditioned medium from transfected cells were also analyzed by westernblotting using antisera from rabbits immunized with PEP2 and PEP3 (FIG. 14). An anti V5 blot was run in parallel as a positive control and the same band pattern was observed as described above. From the PEP2 and PEP3 blot, it is evident that bands were detected of exactly the same size as with anti V5 antibody. Also, it should be noted that there was no signal from the mock transfected negative control. It was also possible to detect Neublasmin-V5 with PEP1 antisera (data not shown). Together these data demonstrates that Neublasmin-V5 protein can be produced and secreted into the medium. In addition, it is evident that the signal peptide can be cleaved off and Neublasmin cleaved internally.

### Example 12

### Detection of Neublasmin Peptides In Vivo (Immunohistochemistry)

Paraffin embedded adult human testis sections were micro wave treated in 5% urea and endogenous peroxidase were quenched by subsequent treatment with 0.5% H₂O₂ for 10 minutes. Sections were washed 1×2 min in water, 3×2 min in TBS and blocked with 2% goat serum (95-6143B, Zymed) for half an hour. Sections were incubated over night at 4° C. with affinity purified PEP1 or PEP3 antibody diluted 1:100. The following day, sections were adjusted to room temperature for one hour and washed with TBS 3×2 min followed by incubation with biotinylated Goat anti Rabbit IgG (95-6143B, Zymed) for half an hour. Sections were washed in TBS 3×2 min, incubated with peroxidase-conjugated Streptavidin (95-6143B, Zymed) for 10 minutes, washed in TBS 3×2 min and finally developed with AEC (00-1111, Zymed) for 20 minutes. Slides were washed with running tap water and mounted with Aquamont mountant (BDH Chemicals).

PEP1 and PEP3 antisera were selected for affinity purification to have antibodies against both the N- and C-terminal of Neublasmin. The purified antibodies were used for immunohistochemistry on testis sections as seen in FIG. 15. In the figure, normal tubuli are shown and it is evident that both antibodies show a very similar staining pattern, demonstrating that Neublasmin protein is produced in vivo. More specifically Neublasmin is localized to the late spermatids/spermatozoa and may therefore play an important role in fertilisation. The expression in germinal cells until meiotiske divisions is low, but then there is a very high expression in post-meiotiske haploid spermatids. The expression seems to peak in very late spermatids/spermatozoa. Neublasmin peptides may therefore have a function down-stream from the testicles, e.g. in epidydimis, spermatic duct, and/or during fertilisation. On the cellular level, it is clear that staining is excluded from the nucleus and instead is localised to the endoplasmatic reticulum and Golgi apparatus as expected for a secreted protein.

1. A Neublasmin peptide selected from the group consisting of
i) a peptide having an amino acid sequence selected from the group consisting of SEQ ID No 26, 27, 28, 29, 30, 31 32, 33, 34, and 35;
ii) a bioactive peptide having at least 95% sequence identity to a peptide selected from the group consisting of SEQ ID No 26, 27, 28, 29, 30, 31 32, 33, 34, and 35;
iii) a bioactive fragment of at least 15 contiguous amino acids of a peptide selected from the group consisting of SEQ ID No 26, 27, 28, 29, 30, 31 32, 33, 34, and 35;
iv) a peptide obtainable by pro-convertase-4 cleavage of a polypeptide having an amino acid sequence selected from the group consisting of SEQ ID No. 9, 10, 14, 18, and 25; and
v) a mature polypeptide obtainable from the culture medium of a mammalian cell expressing a polynucleotide encoding a polypeptide having an amino acid sequence selected from the group consisting of SEQ ID No. 2, 4, 5, 7, 8, 12, 13, 16, 17, 23, and 24.

2. The polypeptide of claim 1, selected from the group consisting of
i) a peptide having an amino acid sequence selected from the group consisting of SEQ ID No 26, 27, 30, 32, and 34;
ii) a bioactive peptide having at least 95% sequence identity to a peptide selected from the group consisting of SEQ ID No 26, 27, 30, 32, and 34; and
iii) a bioactive fragment of at least 15 contiguous amino acids from a peptide selected from the group consisting of SEQ ID No 26, 27, 30, 32, and 34.

3. The polypeptide of claim 1, selected from the group consisting of
i) a peptide having an amino acid sequence selected from the group consisting of SEQ ID No 28, 29, 31, 33, and 35;
ii) a bioactive peptide having at least 95% sequence identity to a peptide selected from the group consisting of SEQ ID No 28, 29, 31, 33, and 35; and
iii) a bioactive fragment of at least 15 contiguous amino acids from a peptide selected from the group consisting of SEQ ID No 28, 29, 31, 33, and 35.

4. The polypeptide of claim 1, selected from the group consisting of Neublasmin peptides having an amino acid sequence selected from the group consisting of SEQ ID No. 26, 27, 28, and 29.

5. The polypeptide of claim 1, selected from the group consisting of Neublasmin peptides having an amino acid sequence selected from the group consisting of SEQ ID No. 28 and 29.

6. The polypeptide of claim 1, selected from the group consisting of Neublasmin peptides having an amino acid sequence selected from the group consisting of SEQ ID No. 26 and 27.

7. The polypeptide according to claim 1, further comprising an affinity tag, such as a polyhis tag, a GST tag, a HA tag, a Flag tag, a C-myc tag, a HSV tag, a V5 tag, a maltose binding protein tag, a cellulose binding domain tag.

8. A nucleic acid comprising a nucleotide sequence coding for a Neublasmin peptide according to claim 1, fused to a signal peptide coding sequence.

9. A method of producing a polypeptide of claim 1, comprising expressing in a cell a nucleic acid coding for a polypeptide of claim 1.

10. The method of claim 9, wherein the cell is a mammalian cell.

11. A method of producing a polypeptide of claim 1, comprising chemical synthesis using custom made peptides.

12. The method of claim 11, wherein the synthesis comprises solid phase peptide synthesis or solution phase peptide synthesis.

13. A pharmaceutical composition comprising the polypeptide of claim 1 and a pharmaceutically acceptable carrier.

14. A method of treating a pathological condition in a subject comprising administering to an individual in need thereof a therapeutically effective amount of the polypeptide of claim 1, wherein the pathological condition is a disorder associated with the nervous system.

15. The method of claim 14, wherein the disorder associated with the nervous system is a CNS disorder.

16. The method of claim 14, wherein the disorder associated with the nervous system is a disease related to testis.

17. The method of claim 16, wherein the disease related to testis is male sterility, male infertility, impotence, erectile dysfunction, cancer, or germ cell tumours.

18. The method of claim 14, wherein the subject is a human being.

19. An antibody generated against a polypeptide of claim 1.

20. The antibody of claim 19, generated against a Neublasmin C-terminal peptide.

21. The antibody of claim 19, generated against a Neublasmin N-terminal peptide.

22. The antibody of claim 19, generated against a peptide having the amino acid sequence of SEQ ID NO 36, 37, or 38.

23. The antibody of claim 19, wherein the polypeptide is coupled to a carrier protein for immunisation.

24. The antibody of claim 19, being selected from the group consisting of: polyclonal antibodies, monoclonal antibodies, humanised antibodies, single chain antibodies, recombinant antibodies.

25. An immunoconjugate comprising the antibody of claim 19 and a conjugate selected from the group consisting of: a cytotoxic agent such as a chemotherapeutic agent, a toxin, or a radioactive isotope; a member of a specific binding pair, such as avidin or streptavidin or an antigen; an enzyme capable of producing a detectable product.

26. A method for determining the presence of or predisposition to a disease associated with altered levels of the polypeptide of claim 1 in a first mammalian subject, the method comprising:
a) measuring the level of expression of the polypeptide in an isolated biological sample from the first mammalian subject; and
b) comparing the amount of the polypeptide in the sample of step (a) to the amount of the polypeptide present in an isolated biological control sample from a second mammalian subject known not to have, or not to be predisposed to, the disease,
wherein an alteration in the expression level of the polypeptide in the first subject as compared to the control sample indicates the presence of or predisposition to the disease.

27. A method of identifying an agent that binds to the polypeptide of claim 1, the method comprising:
(a) introducing the polypeptide to the agent; and
(b) determining whether the agent binds to the polypeptide.

28. The method of claim 27, wherein the agent is a cellular receptor or a downstream effector.

29. The method of claim 27, wherein the agent is identified using yeast two hybrid screening.

30. A method for determining the presence of or predisposition to a disease associated with altered levels of a secreted Neublasmin polypeptide in a first mammalian subject, the method comprising:
a) measuring the level of expression of the polypeptide in an isolated biological sample from the first mammalian subject; and
b) comparing the amount of the polypeptide in the sample of step (a) to the amount of the polypeptide present in an isolated biological control sample from a second mammalian subject known not to have, or not to be predisposed to, the disease,
wherein an alteration in the expression level of the polypeptide in the first subject as compared to the control sample indicates the presence of or predisposition to the disease,
wherein the disease selected from the group consisting of male infertility, carcinoma in situ, seminoma and non-seminoma including embryonal carcinoma, teratoma, and teratocarcinoma.

31. The method of claim 30, wherein the polypeptide is selected from the group consisting of
a) a mature form of the amino acid sequence selected from the group consisting of SEQ ID No. 9, 10, 14, 18, and 25;
b) a variant of a mature form of the amino acid sequence selected from the group consisting of SEQ ID No. 9, 10, 14, 18, and 25, wherein the variant has at least 95% sequence identity to said SEQ ID No.;
c) the amino acid sequence selected from the group consisting of SEQ ID No. 2, 4, 5, 7, 8, 12, 13, 16, 17, 23, and 24;
d) a variant of the amino acid sequence selected from the group consisting of SEQ ID No. 2, 4, 5, 7, 8, 12, 13, 16, 17, 23, and 24, wherein the variant has at least 95% sequence identity to said SEQ ID No.;
e) a mature polypeptide obtainable from the culture medium of a mammalian cell expressing a polynucleotide encoding a polypeptide having an amino acid sequence selected from the group consisting of SEQ ID No. 2, 4, 5, 7, 8, 12, 13, 16, 17, 23, and 24; and
f) a fragment of at least 20 contiguous amino acids of any of a) through d).

32. The method of claim 31, wherein the isolated biological sample is selected from the group consisting of cells, tissue, blood, blood serum, blood plasma, lymph, and sperm.

33. The method of claim 32, wherein the isolated biological sample is sperm, blood, blood serum, or blood plasma.

34. The method of claim 31, wherein the disease is carcinoma in situ, seminoma and non-seminoma including embryonal carcinoma, teratoma, and teratocarcinoma.

35. The method of claim 31, wherein the disease is carcinoma in situ.

36. The method of claim 31, wherein the disease is male infertility.

37. The method of claim 31, wherein the polypeptide is selected from the group consisting of polypeptides having the amino acid sequence of SEQ ID NO 2, 4, 5, 7, 8, 9, 10, and fragments of at least 20 contiguous amino acid of any of these, SEQ ID NO 26, 27, 28, and 29 and fragments of at least 15 contiguous amino acids of any of these.

38. The method of claim 31, wherein the secreted polypeptide is measured using an antibody.

39. The method of claim 38, wherein the antibody is directed against a peptide having the amino acid sequence of SEQ ID NO 36, 37, or 38.

40. A method for determining the presence of or predisposition to a disease associated with altered levels of a Neublasmin nucleic acid molecule in a first mammalian subject, the method comprising:
a) measuring the amount of the nucleic acid in an isolated biological sample from the first mammalian subject; and
b) comparing the amount of the nucleic acid in the sample of step (a) to the amount of the nucleic acid present in an isolated biological control sample from a second mammalian subject known not to have or not be predisposed to, the disease;
wherein an alteration in the level of the nucleic acid in the first subject as compared to the control sample indicates the presence of or predisposition to the disease,
wherein the disease selected from the group consisting of male infertility, carcinoma in situ, seminoma and non-seminoma including embryonal carcinoma, teratoma, and teratocarcinoma.

41. The method of claim 40, wherein the Neublasmin nucleic acid comprises a nucleotide sequence selected from the group consisting of
a) the nucleotide sequence selected from the group consisting of SEQ ID No. 1, 3, 6, 11, 15, 19, 20, 21, and 22;
b) a nucleotide sequence having at least 95% sequence identity to a nucleotide sequence selected from the group consisting of SEQ ID No. 1, 3, 6, 11, 15, 19, 20, 21, and 22;
c) a nucleic acid sequence of at least 60 contiguous nucleotides of a sequence selected from the group consisting of SEQ ID No. 1, 3, 6, 11, 15, 19, 20, 21, and 22;
c) the complement of a nucleic acid capable of hybridising with nucleic acid having the sequence selected from the group consisting of SEQ ID No.: 1, 3, 6, 11, 15, 19, 20, 21, and 22 under conditions of high stringency; and
d) the nucleic acid sequence of the complement of any of the above.

42. A method of treating male infertility comprising administering to a human subject a therapeutically effective amount of a neublasmin polypeptide.

43. The method of claim 42, wherein the neublasmin polypeptide is selected from the group consisting of polypeptides having the amino acid sequence of SEQ ID NO 2, 4, 5, 7, 8, 9, 10, and fragments of at least 20 contiguous amino acid of any of these, SEQ ID NO 26, 27, 28, and 29 and fragments of at least 15 contiguous amino acids of any of these.

44. The method of treating a population of cells comprising administering a neublasmin polypeptide as a growth factor to a mammalian cell culture in vitro.

45. The method of claim 44, for stimulating sperm cells in vitro.

46. The method of claim 44, for in vitro fertilisation.

47. The method of claim 44, for insemination.

48. The method of claim 44, wherein the neublasmin polypeptide is selected from the group consisting of polypeptides having the amino acid sequence of SEQ ID NO 2, 4, 5, 7, 8, 9, 10, and fragments of at least 20 contiguous amino acid of any of these, SEQ ID NO 26, 27, 28, and 29 and fragments of at least 15 contiguous amino acids of any of these.

49. A method of treatment of male infertility comprising administering to an individual in need thereof a therapeutically effective amount of a neublasmin polypeptide.

50. The method of claim 49, wherein the neublasmin polypeptide is selected from the group consisting of polypeptides having the amino acid sequence of SEQ ID NO 2, 4, 5, 7, 8, 9, 10, and fragments of at least 20 contiguous amino acid of any of these, SEQ ID NO 26, 27, 28, and 29 and fragments of at least 15 contiguous amino acids of any of these.

51. An ELISA kit comprising
i) a first antibody capable of forming a species specific linkage to a first Neublasmin epitope,
ii) a surface for immobilisation of antibody or Neublasmin sample;
iii) a detectable label capable of being linked to a Neublasmin polypeptide through a species specific linkage; and
iv) buffers and reagents.

52. The kit of claim 51, wherein the detectable label is capable of being linked to a second Neublasmin epitope through at least one species specific linkage, said at least one species specific linkage comprising a second antibody capable of binding a second Neublasmin epitope.

53. The kit of claim 51, wherein the detectable label is capable of being linked to the first antibody through at least one species specific linkage.

54. A kit for detection of a Neublasmin polypeptide, comprising
i) an antibody capable of binding to a Neublasmin epitope,
ii) a surface for immobilisation of antibody or Neublasmin polypeptide,
iii) a labelled Neublasmin polypeptide comprising said Neublasmin epitope, and
iv) buffers and reagents.

55. A kit according to claim 54, wherein the labelled Neublasmin polypeptide is radioactively labelled.

56. A kit according to claim 54, wherein the labelled Neublasmin polypeptide is covalently linked to an enzyme.

57. A kit for detection of a Neublasmin mRNA comprising either a labelled nucleic acid probe capable of hybridising in situ with a Neublasmin mRNA, or a pair of Neublasmin primers for quantitative amplification of a Neublasmin cDNA.
